(19) United States
(12) Patent Application Publication
(10) Pub. No.: US 2008/0058297 A1
(43) Pub. Date: Mar. 6, 2008

(54) Heterocyclic Compounds For Preventing And Treating Disorders Associated With Excessive Bone Loss

(75) Inventors: Mitsunori Ono, Lexington, MA (US); Lijun Sun, Harvard, MA (US); Yumiko Wada, Billerica, MA (US); Keizo Koya, Chestnut Hill, MA (US); Masazumi Nagai, Lexington, MA (US)

Correspondence Address: EDWARDS ANGELL PALMER & DODGE LLP, P.O. BOX 55874, BOSTON, MA, 02205, US

(73) Assignee: Synta Pharmaceuticals Corp., Lexington, MA (US)

(21) Appl. No.: 10/561,025

(22) PCT Filed: May 28, 2004
(86) PCT No.: PCT/US04/17064

§ 371 Date: May 19, 2006

Related U.S. Application Data
(60) Provisional application No. 60/474,550, filed on May 29, 2003.
(60) Provisional application No. 60/474,502, filed on May 29, 2003.
(60) Provisional application No. 60/474,410, filed on May 29, 2003.

(51) Int. Cl. A61K 31/56; A61K 31/496; A61K 31/5377; C12N 5/02; C07D 295/00; A61K 31/541

(52) U.S. Cl. 514/171; 435/375; 514/227.8; 514/235.8; 514/252.16; 544/123; 544/370; 544/60

(57) ABSTRACT

This invention relates to pyrimidine compounds of formula (I), formula (I′), and formula (I″):

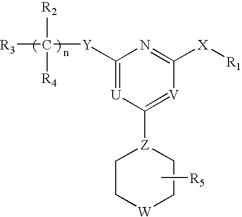

and pharmaceutically acceptable salts, solvates, clathrates, and prodrugs thereof, wherein R₁, R₂, R₃, R₄, R₅, U, V, W, X, Y, Z, and n are defined herein. This invention also relates to compositions comprising these compounds and methods for using them. The compounds and compositions of this invention are useful to treat or prevent disorders associated with excessive bone loss, including, without limitation periodontal disease, non-malignant bone disorders (such as osteoporosis, Pagers-disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

This application claims priority from U.S. Provisional Application Nos. 60/474,502; 60/474,550; and 60/474,410, filed May 29, 2003, which are incorporated herein by reference in their entirety.

### FIELD OF THE INVENTION

The invention relates to biologically active pyrimidines, triazines, and bicyclic compounds, compositions comprising those compounds and methods for their use. The compounds and compositions of this invention inhibit osteoclast formation and may be used to prevent and treat disorders associated with excessive bone loss.

### BACKGROUND OF THE INVENTION

Osteoclasts are unique multinucleated cells within bone that are responsible for bone degradation and resorption. These are the only cells in the body known to be capable of this function. Osteoclasts have a high capacity for the synthesis and storage of enzymes, including acid hydrolases and carbonic anhydrase isoenzyme II. Osteoclasts share phenotypic characteristics with circulating monocytes and tissue macrophages (N. Kurihara et al., Endocrinology 126: 2733-41 (1990); G. Hattersley et al, Endocrinology 128: 259-62 (1991)). These cells are derived from mononuclear precursors that are the progeny of stem-cell populations located in the bone marrow, spleen, and liver. Proliferation of these stem-cell populations produces osteoclastic precursors, which migrate via vascular routes to skeletal sites. These cells then differentiate and fuse with each other to form osteoclasts, or alternatively, fuse with existing osteoclasts.

Osteoclast activation is generally thought to involve release of organic acids and membrane-bound packages of enzymes onto the bone surface. This requires elaboration in proximity with the bone surface of a specialized region of the plasma membrane. In this region, the osteoclast's prepackaged, membrane-bound enzymes can fuse with the plasma membrane and be released onto the bone surface in a confined extracellular space. Degradation of the inorganic and organic tissue occurs in this area. The products of resorption are then taken up via endocytosis for additional intracellular processing within cytoplasmic vacuoles. During bone resorption, osteoclasts remove both the mineral and organic components of bone (H. C. Blair et al., J. Cell Biol. 102: 1164 (1986)). The mineral phase is solubilized by acidification of the sub-osteoclastic lacuna, thus allowing dissolution of hydroxyapatite (G. Vaes, Clin. Orthop. Relat. 231: 239 (1988)).

The regulation of osteoclastic formation and activity is only partly understood but it is known that excessive bone resorption by osteoclasts contributes to the pathology of many human diseases associated with excessive bone loss, including periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers). The following paragraphs provide a description of some of the major disease categories associated with excessive bone loss.

Osteoporosis is a major skeletal disease characterized by low bone mass, architectural deterioration, and an increased risk of fracture, especially of the hip, spine, and wrist. Osteoporosis is implicated in more than 1.5 million fractures per year in the United States. 10 million individuals in the U.S. are estimated to already have the disease and almost 34 million more are estimated to have low bone mass, placing them at increased risk for osteoporosis.

There is evidence of significant mortality and morbidity associated with osteoporosis. The cost of osteoporotic fractures in the United States is over $10 billion annually. As peak bone mass is attained (usually between the ages of 35 and 40 in humans) an imbalance occurs between the processes of bone formation by osteoblasts and bone resorption by osteoclasts. The amount of bone resorbed by osteoclasts is not entirely replaced by osteoblasts. In older women, the speed of bone remodeling (bone turnover) increases after menopause. The outcome is accelerated loss of bone and a negative calcium balance.

Although there is no cure for osteoporosis, several medications have been approved to prevent and/or treat osteoporosis, including bisphosphonates estrogens and progestins, parathyroid hormone and portions thereof, and selective estrogen receptor modulators (SERMs). Treatments under investigation include parathyroid hormones, sodium fluoride, vitamin D metabolites, and other bisphosphonates and selective estrogen receptor modulators. None of these therapies is entirely effective in treating or preventing osteoporosis or ameliorating the symptoms of the disease.

Paget's disease of bone is the second most common bone disease in the US after osteoporosis. It is characterized by an abnormal formation of bone tissue that results in weakened and deformed bones. Paget's disease affects 1-3% of people over 50 years of age, and over 10% of people over 80 years of age. Paget's disease can affect one or more bones in the body. Most often, the pelvis, bones in the skull, the long bones (the large bones that make up the arms and legs), and the collarbones are affected by Paget's disease. In addition, the joints between bones (the knees or elbows, for example) can develop arthritis because of this condition. The underlying cause of Paget's disease is not known.

Paget's disease is most often treated with drug therapy, Including nonsteroidal anti-inflammatory drugs to reduce bone pain, hormone treatment and/or bisphonate treatment. The hormone calcitonin, which is made naturally by the thyroid gland, is commonly used to treat Paget's disease. This compound decreases the amount of bone resorption. Although calcitonin is effective in slowing the progression of Paget's disease, the favorable effects of the drug do not continue for very long once drug administration is stopped. In addition, certain unwanted side effects can occur. Nausea and flushing are the most common side effects and have been found in 20-30% of individuals taking calcitonin. Vomiting, diarrhea, and abdominal pain can also occur. A form of calcitonin taken nasally tends to cause fewer side effects, but requires higher doses because less of the drug reaches the diseased bone. In contrast, the bisphosphonate group of drugs binds directly to bone. Once bound, these drugs inhibit bone loss by reducing the action of bone cells that normally degrade bone during the remodeling process. Because of its long acting activity, bisphosphonates are currently considered the treatment of choice for Paget's disease. Specific bisphosphate drugs suitable for the treatment of Paget's disease are etidronate, pamidronate, alendronate, clodronate, and tiludronate. The main side effects of these drugs include a flu-like reaction (pamidronate), gastrointestinal disturbances (alendronate, clodronate), and abnormal bone formation (etidronate, when taken in high doses) (S. Krane “Paget's Disease of Bone.”. In Harrison's. Principles of Internal Medicine, edited by Anthony S. Fauci, et al. New York: McGraw Hill, 2266-69 (1998)).

Loss of ovarian function following menopause often produces a progressive loss of trabecular bone mass that can eventually lead to osteoporosis and other bone diseases. The bone loss is due at least in part to the decreased elaboration by support cells of osteoclastogenic cytokines such as IL-1, tumor necrosis factor and IL-6, all of which are negatively regulated by estrogens. For example, estrogen has been shown to negatively regulate NF-κB and macrophage colony stimulating factor (M-CSF)-induced differentiation of mononuclear precursors into multinucleated osteoclasts (N. Shevde et al., Proc Natl Acad Sci USA 97: 7829-34 (2000)). In this case, estrogen blocks the transcription of M-CSF-induced proteins and forms osteoblasts by downregulating the expression of osteoclastogenic cytokines.

Bone loss in the oral cavity and periodontal disease are also significant problems in the United States. Interdisciplinary attention has focused on possible relationship between osteoporosis and oral bone loss (Proceedings of the Workshop on Oral Bone Loss and Osteoporosis, Leesburg, Va., Aug. 26-28, 1992, in J. Bone Miner. Res. 8, Supplement 2, 1993). Periodontal disease (periodontitis) is characterized by loss of bone and soft tissue attachment. The response to the formation of microbial plaque is an inflammation of the gingiva and the resulting breakdown of tissues. This causes the formation of an opening along the tooth surface known as the “periodontal pocket”. The bone remodeling that occurs in periodontal disease is typically localized to the alveolar bone. The mechanism of alveolar bone loss in periodontal disease is believed to be the same basic mechanism as is responsible for bone loss associated with other types of inflammatory conditions. It has been presumed that accumulations of chronic inflammatory cells generate inflammatory cytokines and local mediators that are responsible for enhanced osteoclastic resorption and inhibition of repair or new bone formation at the sites of resorption. For instance, inflammatory mediators, such as prostaglandins (Offenbacher et al., J. Periodont. Res. 21: 101-112 (1986)) have been associated with active progression of periodontitis. A prostaglandin antagonist has been shown to inhibit osteoclast formation in cell culture (Inoue et al., J. Endocrinol. 161: 231-36 (1999)). IL-1, another mediator of inflammation, has been found in gingival crevicular fluid during inflammation (Charon et al., Infect. Immun. 38: 1190-95 (1982)). IL-12 alone and in synergy with IL-18 has been shown to inhibit osteoclast formation (Horwood et al., J Immun. 166(8): 4915-21 (2001)).

Primary hyperparathyroidism is a hormonal problem which occurs when one or more of the parathyroid glands produces excess parathyroid hormone. When this occurs, blood calcium is elevated and bones may lose calcium. At present, there is no approved medical therapy for primary hyperparathyroidism and surgery is often the only available option.

Fibrous dysplasia is a chronic disorder of the skeleton which causes expansion of one or more bones due to abnormal development of fibrous tissue within the bone. Any bone can be affected, and involvement can be in one or several bones. Though many bones can be affected at once, fibrous dysplasia does not spread from one bone to another. At present there are no approved medical therapies.

Osteoclast activity resulting in excessive bone loss has also been implicated in various forms of arthritis (such as septic arthritis, osteoarthritis, juvenile arthritis and rheumatoid arthritis). For example, it has been shown that osteoclastic activity is responsible for the focal bone erosions in areas of pannus invasion which are the hallmark of established rheumatoid arthritis. (E. Gravallese et al., Arthritis Res 1(Suppl 1):S37 (1999)). Drugs that are used in the treatment of arthritis tend to address the inflammation associated with the disease rather than the cause. These treatments include steroids and non-steroidal anti-inflammatory drugs (including COX-II inhibitors).

Osteopetrosis is an inherited defect characterized by a failure of normal bone resorption (modeling) and, as a result, excessive bone accumulation throughout the skeleton. Osteopetrosis occurs in a number of species, including man. The disease represents a heterogeneous group of bone disorders both in animal species demonstrating these defects and in the infantile malignant forms of osteopetrosis. The skeletal sclerosis and reduced bone marrow resorption in certain animal species have been shown to be due to defective osteoclasts. The skeletal abnormalities associated with osteopetrosis lead to a number of problems, including anemia, infection, optic atrophy, deafness and various neuropathies.

Presently available forms of treatment for osteopetrotic children include bone marrow transplantation and interferon-gamma therapy. Bone marrow transplantation is not available to most osteopetrotic children and not all children who receive bone marrow transplants respond favorably. Interferon-gamma therapy has demonstrated moderate success in improving osteoclast function (Key et al., J. Pediatr. 121: 119-24 (1992)) but requires high doses and extensive clinical monitoring to avoid the potential toxic effects associated with this cytokine.

The study of osteopetrosis has been facilitated by the existence of a number of osteopetrotic animal mutations. For a discussion of such mutations, see Marks, Clinical Orthopedics, 180: 239-263 (1984). The “incisors-absent” (I) (Greep, J. Hered. 32: 397 (1941)) and osteopetrotic (op) (Moutier et I., Animal 6: 87 (1973)) rat mutations, as well as certain other animal congenital osteopetrotic mutations, have been shown to respond to spleen cell or bone marrow transplantation (Marks, Am. J. Anat. 146: 331 (1976); Milhaud et al., C.R. Acad. Sci. Paris 280: 2485 (1975)), thereby paving the way for the first successful reported treatment of congenital human osteopetrosis by Ballet et al.; Lancet 2: 1137 (1977). Hence these mutations provide an acceptable corollary to human osteopetrosis.

Inflammation-mediated bone loss is a problem of major clinical and economic significance. Inflammation-mediated bone loss occurs in numerous diseases such as osteoporosis, periodontal disease, osteoarthritis, and rheumatoid arthritis. Studies attempting to identify the factor(s) which mediate such bone loss have implicated various immune cell products, i.e. cytokines and growth factors. For a recent short review see Mundy, J. Bone Miner. Res. 8, Supplement 2: S505-S510 (1993). It has been suggested that the major mediators likely involved include interleukin 1, tumor necrosis factor-alpha, lymphotoxin, interleukin 6, prostaglandins of the E series, leukotrienes, lipopolysaccharide, transforming growth factor-beta, and the colony-stimulating factors. But no studies have provided conclusive evidence of cytokines' pathogenic role in bone degradation. Some studies have yielded conflicting data. The production of a particular cytokine may be elevated in some patients but not in others, yet all have the same disease and demonstrate similar amounts of bone loss. Based on these studies, the treatment strategies designed to help prevent or treat the bone loss associated with inflammation have either been ineffective or have shown limited therapeutic efficacy in a subset of patients with a specific disease.

In view of the above, there remains a need for new agents that inhibit formation of osteoclasts for use in preventing and treating disorders associated with excessive bone loss.

### SUMMARY OF THE INVENTION

This invention meets the needs described above by providing compounds and compositions that inhibit the formation of osteoclasts and methods for using them. These compounds and compositions are particularly useful for treating or preventing disorders associated with excessive bone loss. Such disorders include, without limitation, periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

The invention features heterocylic compounds of formula (I):

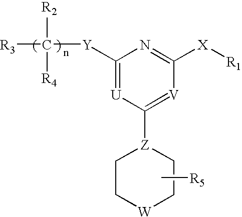

and pharmaceutically acceptable salts, solvates, clathrates, and prodrugs thereof, wherein:

R₁ is

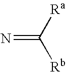

[sometimes referred to hereinafter as NC(R^{a}R^{b})], aryl, or heteroaryl; each of R₂ and R₄, independently, is R^{c}, halogen, nitro, cyano, isothionitro, SR^{c}, or OR^{c}; or R₂ and R₄, taken together, is carbonyl; R₃ is R^{c}, alkenyl, alkynyl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d}; R₅ is H or alkyl; n is 0, 1, 2, 3, 4, 5, or 6; X is O, S, S(O), S(O₂), or NR^{c}; Y is a covalent bond, CH₂, C(O), C═N—R^{c}, C═N—OR^{c}, C═N—SR^{c}, O, S, S(O), S(O₂), or NR^{c}; Z is N or CH; one of U and V is N, and the other is CR^{c}; and W is O, S, S(O), S(O₂), NR^{c}, or NC(O)R^{c}C; in which each of R^{a} and R^{b}, independently, is H, alkyl, aryl, heteroaryl; and each of R^{c} and R^{d}, independently, is H, alkyl, aryl, heteroaryl, cyclyl, heterocyclyl, or alkylcarbonyl. Note that unless otherwise depicted, the left atom shown in any substituted group described above is the one closest to the pyrimidine ring. Also note that when n is 2 or greater, the just-described pyrimidine compound may have two or more different C(R²R⁴) moieties, or when there are more than one R-containing substituted groups in a pyrimidine compound, the R^{c} moieties can be the same or different. The same rules apply to other similar situations. Further note that R^{c} can be a monovalent or bivalent substitutent.

The invention features compounds of formula (I′):

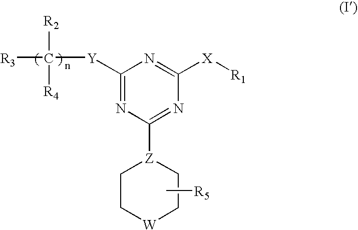

and pharmaceutically acceptable salts, solvates, clathrates, and prodrugs thereof, wherein:
wherein R₁ is

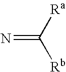

[sometimes referred to hereinafter as NC(R^{a}R^{b})], aryl, or heteroaryl; each of R₂, R₄, and R₅, independently, is R^{c}, halogen, nitro, nitroso, cyano, azide, isothionitro, SR^{c}, or OR^{c}; R₃ is R^{c}, alkenyl, alkynyl, aryl, heteroaryl, cyclyl, heterocyclyl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d}; n is 0, 1, 2, 3, 4, 5, 6, or 7; X is O, S, S(O), S(O₂), or NR^{c}; Y is a covalent bond, CH₂, C(O), C═N—R^{c}, C═N—OR^{c}, C═N—SR^{c}, O, S, S(O), or S(O₂); Z is N; and W is O, S, S(O), S(O₂), NR^{c}, or NC(O)R^{c}; in which each of R^{a} and R^{b}, independently, is H, alkyl, aryl, heteroaryl; and each of R^{c} and R^{d}, independently, is H, alkyl, or alkylcarbonyl. Note that the left atom shown in any substituted group described above is closest to the tirazine ring. Also note that when n is 2 or greater, the just-described triazine compound may have two or more different C(R²R⁴) moieties. The same rule applies to other similar situations.

The invention features compounds of formula (I″):

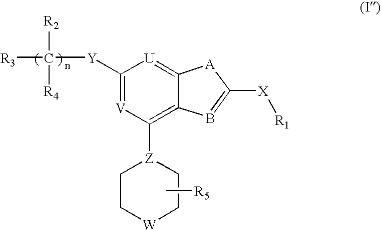

and pharmaceutically acceptable salts, solvates, clathrates, and prodrugs thereof, wherein:
R₁ is aryl or heteroaryl; each of R₂ and R₄, independently, is H, halogen, CN, alkyl, OR^{a}, or NR^{a}R^{b}; R₃ is H, halogen, CN, alkyl, alkenyl, alkynyl, aryl, heteroaryl, cyclyl, heterocyclyl, OR^{a}, OC(O)R^{a}, OC(O)NR^{a}R^{b}, NR^{a}R^{b}, R^{a}C(O)R^{b}, NR^{a}S(O)R^{b} NR^{a}S(O)₂R^{b}, NR^{a}C(O)NR^{b}R^{c}, NR^{a}C(S)NR^{b}R^{c}, NR^{a}C(NR^{b})NR^{c}R^{d}, NR^{a}C(O)R^{b}, S(O)NR^{a}R^{b}, S(O)₂NR^{a}R^{b}, S(O)R^{a}, S(O)₂R^{a}, C(O)R^{a}, C(O)OR^{a}, or C(O)NR^{a}R^{b}; R₅ is H or alkyl; n is 0, 1, 2, 3, 4, 5, or 6; A is O, S, S(O), S(O)₂, or NR^{e}; B is N or CR^{f}; X is O, S, S(O), S(O)₂, NR^{e}, or C(O); Y is a covalent bond, C(O), C═NR^{a}, O, S, S(O), S(O)₂, or NR^{e}; Z is N or CH; each of U and V, independently, is N or CR; and W is O, S, or NR^{e}; in which each of R^{a}, R^{b}, R^{c}, and R^{d}, independently, is H, alkyl, aryl, heteroaryl, cyclyl, or heterocyclyl; R^{e} is H, alkyl, aryl, acyl, or sulfonyl; and R₁ is H, alkyl, aryl, acyl, sulfonyl, alkoxyl, amino, ester, amide, CN, or halogen; and provided that if each of U and V is N, Y is a covalent bond, n is 0, then R₃ is H, CN, alkyl, alkenyl, alkynyl, aryl, heteroaryl, cyclyl, OR^{a}, OC(O)R^{a}, OC(O)NR^{a}R^{b}, NR^{a}R^{b}, NR^{a}C(O)R^{b}, NR^{a}S(O)R^{b}, NR^{a}S(O)₂R^{b}, NR^{a}C(O)NR^{b}R^{c}, NR^{a}C(S)NR^{b}R^{c}, NR^{a}C(NR^{b})NR^{c}R^{d}, NR^{a}C(O)OR^{b}, S(O)NR^{a}R^{b}, S(O)₂NR^{a}R^{b}, S(O)R^{a}, S(O)₂R^{a}, C(O)R^{a}, C(O)OR^{a}, or C(O)NR^{a}R^{b}. Note that the left atom shown in any substituted group described above is closest to the aromatic bicyclic ring. Also note that when there are more than one R^{a}-containing substituted groups in a compound of formula (I″), the R^{a} moieties can be the same or different. The same rule applies to other similar situations.

Compounds of formula (I), formula (I′), or formula (I″) and pharmaceutically acceptable salts, solvates, clathrates, and prodrugs thereof and compositions comprising those compounds may be used to treat or prevent disorders associated with excessive bone loss. Such disorders include, without limitation, periodontal disease, non-malignant bone disorders (such as osteoporosis, Page t's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

The compositions of this invention comprise an effective amount of a compound of formula (I), formula (I′), or formula (I″) or a pharmaceutically acceptable salt, solvate, clathrate, or prodrug thereof; and a pharmaceutically acceptable carrier or vehicle. These compositions may further comprise one or more additional active agents. The compositions are useful for treating or preventing the above mentioned disorders.

The invention further encompasses methods for inhibiting osteoclast formation in vitro or in vivo, comprising contacting a pre-osteoclast cell (e.g., a cell capable of forming an osteoclast cell upon differentiation and/or fusion) with an effective amount of a compound of formula (I), formula (I′), or formula (I″) or a pharmaceutically acceptable salt, solvate, clathrate, or prodrug thereof or a pharmaceutical composition comprising an effective amount of a compound of formula (I), formula (I′), or formula (I″) or a pharmaceutically acceptable salt, solvate, clathrate, or prodrug thereof.

The invention further encompasses methods of treating or preventing a disorder associated with excessive bone resorption by osteoclasts in a patient in need thereof, comprising the step of administering to the patient an effective amount of a compound of formula (I), formula (I′), or formula (I″) or a pharmaceutical composition comprising an effective amount of a compound of formula (I), formula (I′), or formula (I″) or a pharmaceutically acceptable salt, solvate, clathrate, or prodrug thereof.

### DETAILED DESCRIPTION OF THE INVENTION

### Definitions

Unless otherwise specified, the below terms used herein are defined as follows:

The term “alkyl” refers to a straight-chained or branched alkyl group containing 1 to 6 carbon atoms. Examples of alkyl groups include methyl (Me), ethyl (Et), n-propyl (Pr), isopropyl (i-Pr), tert-butyl, and n-pentyl. Any carbon in the alkyl group may optionally be substituted with carbonyl (C═O), oxygen (O), sulfur (S), or nitrogen (N).

The term “alkenyl” refers to a straight-chained or branched alkenyl group containing 2 to 6 carbon atoms. Examples of alkenyl groups include vinyl, allyl (2-propenyl), dimethylallyl, and butenyl.

The term “alkynyl” refers to a straight-chained or branched alkynyl group containing 2 to 6 carbon atoms. Examples of alkynyl groups include ethynyl and propargyl.

The term “aryl” refers to a hydrocarbon ring system (monocyclic or bicyclic) having at least one aromatic ring. Examples of aryl moieties include, but are not limited to, phenyl, naphthyl, and pyrenyl.

The term “heteroaryl” refers to a hydrocarbon ring system (monocyclic or bicyclic) having at least one aromatic ring which contains at least one heteroatom (e.g., O, N, or S) as part of the ring system. Examples of heteroaryl moieties include, but are not limited to, pyridinyl, triazolyl, tetrazolyl, pyrimidinyl, thiazolyl, indolyl, and indolizinyl.

The terms “cyclyl” and “heterocyclyl” refer to partially and fully saturated mono- or bi-cyclic rings having from 4 to 14 ring atoms. A heterocyclyl ring contains one or more heteroatoms (e.g., O, N, or S) as part of the ring. Exemplary cyclyl and heterocyclyl rings are cycylohexane, piperidine, piperazine, morpholine, thiomorpholine, 1,4-oxazepane and 1H-pyridin-2-one.

As used herein, the term “halogen” or “halo” means —F, —Cl, —Br or —I.

As used herein, the terms “animal”, “subject” and “patient”, include, but are not limited to, a cow, monkey, horse, sheep, pig, chicken, turkey, quail, cat, dog, mouse, rat, rabbit, guinea pig and human (preferably, a human).

As used herein, the term “lower” refers to a group having up to four atoms. For example, a “lower alkyl” refers to an alkyl radical having from 1 to 4 carbon atoms, and a “lower alkenyl” or “lower alkynyl” refers to an alkenyl or alkynyl radical having from 2 to 4 carbon atoms, respectively

As used herein, the term “sulfanyl” refers to a thio group.

As used herein, the terms “alkyl”, “alkenyl”, “alkynyl”, “aryl”, “heteroaryl”, “cyclyl”, and “heterocyclyl” and other groups that may contain substituents include both the substituted and unsubstituted moieties. The term “substituted” refers to one or more substituents (which may be the same or different), each replacing a hydrogen atom. Examples of substituents include, but are not limited to, halogen, hydroxyl, amino, alkylamino, arylamino, dialkylamino, diarylamino, cyano, nitro, mercapto, carbonyl, carbamido, carbamyl, carboxyl, thioureido, thiocyanato, sulfoamido, C₁-C₆ alkyl, C₁-C₆ alkenyl, C₁-C₆ alkoxy, aryl, heteroaryl, cyclyl, heterocyclyl, wherein alkyl, alkenyl, alkoxy, aryl, heteroaryl cyclyl, and heterocyclyl are optionally substituted with C₁-C₆ alkyl, aryl, heteroaryl, halogen, hydroxyl, amino, mercapto, cyano, or nitro.

As used herein, the term “compound(s) of this invention” and similar terms refer to a compound of formula (I), formula (I′), or formula (I″) or a pharmaceutically acceptable salt, solvate, clathrate, or prodrug thereof.

As used herein, the term “effective amount” means an amount of a compound of this invention sufficient to measurably inhibit formation of osteoclasts a relevant in vitro assay or cause a measurable improvement in an animal model of a particular disease associated with excessive bone loss. Alternatively, an “effective amount” is an amount of a compound of this invention sufficient to confer a therapeutic or prophylactic effect on the treated patient against a disease associated with excessive bone loss. The interrelationship of dosages for animals and humans (based on milligrams per meter squared of body surface) is described in Freireich et al., Cancer Chemother Rep 50: 219 (1966). Body surface area may be approximately determined from height and weight of the patient. See, e.g., Scientific Tables, Geigy Pharmaceuticals, Ardley, N.Y., 1970, 537. An effective amount of the compound when administered orally will typically range from about 0.1 mg/day to about 5000 mg/day (and preferably, about 1 mg/day to about 1000 mg/day and more preferably, about 10 to about 500 mg/day). These amounts may be administered in a single dosage form or may be administered in several (e.g., two to six, preferably two to four and more preferably, two or three) doses per day. Effective amounts will also vary, as recognized by those skilled in the art, depending on the diseases treated, route of administration, excipient usage, and the possibility of co-usage with other therapeutic treatments such as use of other agents.

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, but they may have activity in their unreacted forms. Examples of prodrugs contemplated in this invention include, but are not limited to, analogs or derivatives of compounds of formula (I), formula (I′), or formula (I″) that comprise biohydrolyzable moieties' such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of formula (I), formula (I′), or formula (I″) that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY 172-178, 949-932 (1995) (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “pharmaceutically acceptable salt,” is a salt formed from an acid and a basic group of one of the compounds of formula (I), formula (I′), or formula (I″). Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutarnate, methanesulfonate, ethanesulfonate, benzenesulfonate, p-toluenesulfonate, and pamoate (i.e., 1,1′-methylene-bis-(2-hydroxy-3-naphthoate)) salts. The term “Pharmaceutically acceptable salt” also refers to a salt prepared from a compound of formula (I), formula (I′), or formula (I″) having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. Other pharmaceutically acceptable salts are described in the Handbook of Pharmaceutical Salts. Properties, Selection, and Use (P. Heinrich Stahl and C. Wermuth, Eds., Verlag Helvetica Chica Acta, Zurich, Switzerland (2002)).

As used herein, the term “pharmaceutically acceptable solvate,” is a solvate formed from the association of one or more solvent molecules to one of the compounds of formula (I), formula (I′), or formula (I″). The term solvate includes hydrates (e.g., mono-hydrate, dihydrate, trihydrate, tetrahydrate, and the like).

As used herein, the term “pre-osteoclast cell” is a cell capable of forming an osteoclast cell upon differentiation and/or fusion and includes without limitation, circulating monocytes and tissue macrophages (N. Kurihara et al., Endocrinology 126: 2733-41 (1990)). Without wishing to be bound by theory, pre-osteoclasts are converted to activated osteoclasts in a process thought to involve two factors produced by pre-osteoblasts, M-CSF and ODF. These factors activate certain genes that are needed for the conversion of a pre-osteoclast into an osteoclast.

Carriers and vehicles used in the compositions of this invention must be “acceptable” in the sense of being compatible with the active ingredient of the formulation (and preferably, capable of stabilizing it) and not deleterious to the patient to be treated. For example, solubilizing agents such as cyclodextrins, which form specific, more soluble complexes with the compounds of this invention, or one or more solubilizing agents, can be utilized as pharmaceutical excipients for delivery of the pyrimidine compounds. Examples of other carriers include colloidal silicon dioxide, magnesium stearate, cellulose, sodium lauryl sulfate, and D&C Yellow # 10. Other suitable carriers and vehicles are known to those of ordinary skill in the art. For convenience, the term “carrier” as used herein will encompass all such carriers, adjuvants, diluents, excipients, solvents or other inactive additives. Formulation of the compound to be administered will vary according to the route of administration selected (e.g., solution, emulsion, capsule) and the disease, disorder or condition targeted. Suitable pharmaceutical carriers may contain inert ingredients which do not substantially interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

Further, the compounds of this invention also include their N-oxides. The term “N-oxides” refers to one or more nitrogen atoms, when present in a compound, are in N-oxide form, i.e., N→O.

It should also be noted that when a compound of formula (I), formula (I′), or formula (I″) has more than one R^{a-h}-containing substituents, the R^{a-h} moieties can be the same or different in each instance. The same rule applies to other similar situations where the same designation is used for different variable moieties in the same compound.

Also within the scope of this invention are a composition containing one or more of the compounds described above for use in treating or preventing a disorder associated with excessive bone loss, and the use of such a composition for the manufacture of a medicament for the just-described use.

As used herein, “disorders associated with excessive bone loss”, “disorders associated with excessive osteoclast activity” and similar terms mean a disease, disorder or condition characterized by excessive bone loss. Examples of such disorders include without limitation, periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

As used herein, a racemic mixture means about 50% of one enantiomer and about 50% of is corresponding enantiomer relative to all chiral centers in the molecule. The invention encompasses all enantiomerically-pure, enantiomerically-enriched, diastereomerically pure, diestereomerically enriched, and racemic mixtures of the compounds of formula (I), formula (I′), or formula (I″).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantionmers and diastereomers can also be obtained from diastereomerically- or enantiomerically-pure intermediates, reagents, and catalysts by well known asymmetric synthetic methods.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to a non-human animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least about 80%, preferably at least about 90%, more preferably at least about 95% and even more preferably at least about 98%, of a single compound of the invention by weight of the isolate.

Note that unless otherwise depicted, the leftmost atom shown in any substituted group described herein is closest to the ring or group to which it is attached.

Only those choices and combinations of substituents that result in a stable structure are contemplated. Such choices and combinations will be apparent to those of ordinary skill in the art and may be determined without undue experimentation. In addition, specific substituents that are exemplified, preferred or otherwise noted may be combined to form preferred compounds of this invention.

The invention can be understood more fully by reference to the following detailed description and illustrative examples, which are intended to exemplify non-limiting embodiments of the invention.

### SPECIFIC EMBODIMENTS

The invention relates to compounds and pharmaceutical compositions that are particularly useful for treating or preventing disorders associated with excessive bone loss (including, without limitation, periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of Multiple myeloma and osteolytic bone metastases of breast cancer and other metastatic cancers). The invention further encompasses methods for inhibiting osteoclast formation in vitro or in vivo, comprising contacting a pre-osteoclast cell (e.g., a cell capable of forming an osteoclast cell upon differentiation and/or fusion) with an effective amount of a compound of formulas (I), (I′), and (I″) or a pharmaceutically acceptable salt, solvate, clathrate, or prodrug thereof.

Specific methods and pharmaceutical compositions of the invention comprise a compound of formula (I), formula (I′), or formula (I″) as an active ingredient. In those methods and compositions, an effective amount of a compound of formula (I), formula (I′), or formula (I″) is employed.

Referring to formula (I), a subset of the compounds of this invention is featured by that R¹ is NC(R^{a}R^{b}). In these compounds, U can be N, V can be CH, Z can be N, and W can be O. In addition, X can be NR^{c}; R^{c} can be H, methyl, ethyl, or acetyl; Y can be O, S, or CH₂, and n can be 0, 1, 2, 3, or 4. In some embodiments, R₃ is aryl, heteroaryl (e.g., pyridinyl), OR^{c}, SR^{c}, C(O)OR^{c}, or C(O)NR^{c}R^{d}. In other embodiments, R₃ is

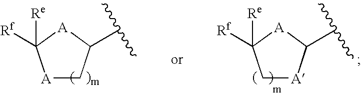

in which each of A and A′, independently, is O, S, or NH; each of R^{e} and R^{f}, independently, is H, alkyl, aryl, or heteroaryl; and m is 1 or 2.

In this subset of pyrimidine compounds, R^{a} or R^{b}, preferably, is

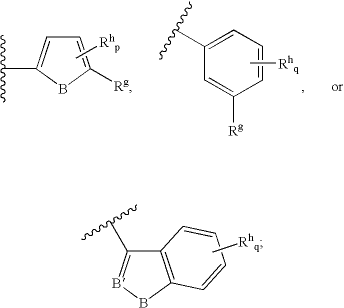

in which B is NRⁱ, O, or S; B′ is N or CRⁱ; R^{g} is H, halogen, CN, alkyl, cyclyl, alkyloxy, alkylcarbonyl, alkyloxycarbonyl, aryloxycarbonyl, heteroaryloxycarbonyl, hydroxyalkyl, alkylamino, or alkylaminocarbonyl; R^{h} is H, halogen, NO₂, CN, alkyl, aryl, heteroaryl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d}; Rⁱ is H, alkyl, or alkylcarbonyl; p is 0, 1, or 2; and q is 0, 1, 2, 3, or 4.

R^{a} or R^{b}, preferably, is

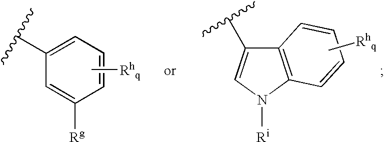

wherein R^{g} is H, methyl, ethyl, propyl, cyclopropyl, methoxy, ethoxy, halogen, methylaminocarbonyl or methoxycarbonyl; R^{h} is F, Cl, CN, methyl, methoxy, ethoxy, OC(O)CH₃, OC(O)C₂H₅, C(O)OH, C(O)OC₂H₅, C(O)NH₂, NHC(O)CH₃, or S(O₂)NH₂; Rⁱ is H, methyl, ethyl, or acetyl; and q is 0, 1, or 2.

Another subset of the pyrimidine compounds of this invention is featured by that R¹ is aryl or heteroaryl. In these compounds, U can be N, V can be CH, Z can be N, and W can be O. In addition, X can be NR^{c}; R^{c} can be H, methyl, ethyl, or acetyl; Y can be O, S, or CH₂, and n can be 0, 1, 2, 3, or 4. In some embodiments, R₃ is aryl, heteroaryl (e.g., pyridinyl, such as pyridin-2-yl or pyridin-3-yl), OR^{c}, SR^{c}, C(O)OR^{c}, or C(O)NR^{c}R^{d}. In other embodiments, R₃ is

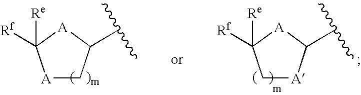

in which each of A and A′, independently, is O, S, or NH; each of R^{e} and R^{f}, independently, is H, alkyl, aryl or heteroaryl; and m is 1 or 2.

In this second subset of pyrimidine compounds, R₁, preferably, is

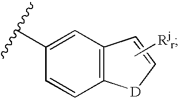

in which D is O, S, or NR^{m}; R^{j} is benzo, halogen, CN, hydroxyl, alkyl, aryl, heteroaryl, alkoxyl, aryloxyl, or heteroaryloxyl; R^{m} is H, alkyl, or alkylcarbonyl; and r is 0, 1, or 2. Preferably, R₁ is

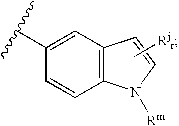

and R^{j} is methyl, ethyl, propyl, or benzo; and r can be 1 or 2.

A third subset of the pyrimidine compounds of formula (I) is featured by that R₁ is NC(R^{a}R^{b}); each of R₂ and R₄ is H; R₃ is H, alkyl, aryl, heteroaryl, cyclyl, heterocyclyl, alkyloxycarbonyl, alkylaminocarbonyl, or alkylcarbonyl; R₅ is H or alkyl; n is 0, 1, 2, 3, 4, 5, or 6; X is NR^{c}; Y is covalent bond, CH₂, C(O), C═N—R^{c}, C═N—OR^{c}, C═N—SR^{c}, O, S, S(O), S(O₂), or NR^{c}; Z is N or CH; one of U and V is N, and the other is CR^{c}; and W is O, S, S(O), S(O₂), NR^{c}, or NC(O)R^{c}; in which each of R^{a} and R^{b}, independently, is H, alkyl, aryl, heteroaryl; and R^{c} is H, alkyl, aryl, heteroaryl, cyclyl, heterocyclyl, or alkylcarbonyl.

In this third subset of pyrimidine compounds, preferably, one of R^{a} and R^{b} is H or alkyl; and the other is aryl or heteroaryl optionally substituted with R^{g} and R^{h}_{q}; R^{g} being halogen, CN, alkyl, alkyloxy, alkylcarbonyl, alkyloxycarbonyl, aryloxycarbonyl, heteroaryloxycarbonyl, hydroxyalkyl, alkylamino, or alkylaminocarbonyl; R^{h} being halogen, CN, hydroxyl, alkyl, aryl, heteroaryl, alkoxyl, aryloxyl, or heteroaryloxyl; and q being 0, 1, 2, 3, or 4. Preferably, one of R^{a} and R^{b} is H or alkyl; and the other is

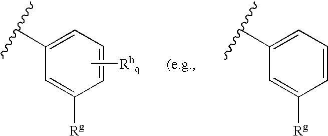

such as 3-methylphenyl);
in which R^{g} is H, alkyl, alkoxyl, methylaminocarbonyl, methoxycarbonyl, or halogen; R^{h} is halogen, CN, hydroxyl, alkyl, aryl, heteroaryl, alkoxyl, aryloxyl, or heteroaryloxyl; and q is 0, 1, 2, 3, or 4.

In some embodiments, X is NH; Y is O; n is 2, or R₃ is heteroaryl (e.g., pyridinyl or 1-oxy-pyridinyl) or heterocyclyl (e.g., 1H-pyridin-2-one). In other embodiments, U is N; V is CH; and R₃ is heteroaryl or heterocyclyl. Preferably, X is NH; Y is O; n is 2; and one of R^{a} and R^{b} is H; and the other is

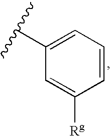

in which R^{g} can be CN, hydroxyalkyl, alkylamino, alkylaminocarbonyl alkyloxycarbonyl (e.g., C(O)OCH₃), or halogen (F, Cl, Br, or I) when R₃ is heteroaryl (e.g., pyridinyl), or R₉ can be halogen (e.g., I), alkyl (e.g., methyl), alkylaminocarbonyl (e.g., methylaminocarbonyl) or alkyloxycarbonyl (e.g., methoxycarbonyl) when R₃ is heterocyclyl (e.g., 1H-pyridin-2-one).

Set forth below are exemplary compounds useful in this invention:
N-{2-[3-(3,4-dimethoxy-phenyl)-propyl]-6-morpholin-4-yl-pyrimidin-4-yl}-N′-(1H-indol-3-ylmethylene)-hydrazine (Compound 1)
N-(2-n-butoxy-6-morpholin-4-yl-pyrimidin-4-yl)-N′-(1H-indol-3-ylmethylene)-hydrazine (Compound 2)
N-(2-(4-hydroxybutyl)-6-morpholin-4-yl-pyrimidin-4-yl)-N′-(1H-indol-3-ylmethylene)-hydrazine (Compound 3)
N-[2-(2-[1,3]dioxan-2-yl-ethyl)-6-morpholin-4-yl-pyrimidin-4-yl]-N′-(1H-indol-3-ylmethylene)-hydrazine (Compound 4)
N-(1H-indol-3-ylmethylene)-N′-[2-(3-methoxy-propyl)-6-morpholin-4-yl-pyrimidin-4-yl]-hydrazine (Compound 5)
3-{4-[N′-(1H-indol-3-ylmethylene)-hydrazino]-6-morpholin-4-yl-pyrimidin-2-ylsulfanyl}-propan-1-ol (Compound 6)
3-{2-[N′-(1H-indol-3-ylmethylene)-hydrazino]-6-morpholin-4-yl-pyrimidin-4-ylsulfanyl}-propan-1-ol (Compound 7)
N-[2-(2,2-dimethyl-[1,3]dioxolan-4-ylmethoxy)-6-morpholin-4-yl-pyrimidin-4-yl]-N′-(1H-indol-3-ylmethylene)-hydrazine (Compound 8)
N-{2-[2-(3,4-dimethoxy-phenyl)-ethoxy]-6-morpholin-4-yl-pyrimidin-4-yl}-N′-(1H-indol-3-ylmethylene)-hydrazine (Compound 9)
N-(1H-indol-3-ylmethylene)-N′-[6-morpholin-4-yl-2-(2-pyridin-2-yl-ethoxy)-pyrimidin-4-yl]-hydrazine (Compound 10)
N-(1H-indol-3-ylmethylene)-N′-[6-morpholin-4-yl-2-(3-pyridin-2-yl-propyl)-pyrimidin-4-yl]-hydrazine (Compound 11)
N-(3-methyl-benzylidene)-N′-[6-morpholin-4-yl-2-(2-pyridin-2-yl-ethoxy)-pyrimidin-4-yl]-hydrazine (Compound 12)
N-(3-ethyl-benzylidene)-N′-[6-morpholin-4-yl-2-(2-pyridin-2-yl-ethoxy)-pyrimidin-4-yl]-hydrazine (Compound 13)
N-(3-methyl-benzylidene)-N′-[6-morpholin-4-yl-2-(3-pyridin-2-yl-propyl)-pyrimidin-4-yl]-hydrazine (Compound 14)
N-[6-morpholin-4-yl-2-(2-pyridin-2-yl-ethoxy)-pyrimidin-4-yl]-N′-(1-m-tolyl-ethylidene)-hydrazine (Compound 15)
N-[1-(1H-indol-3-yl)-ethylidene]-N′-[6-morpholin-4-yl-2-(2-pyridin-2-yl-ethoxy)-pyrimidin-4-yl]-hydrazine (Compound 16)
3-methyl-benzaldehyde O-[6-morpholin-4-yl-2-(2-pyridin-2-yl-ethoxy)-pyrimidin-4-yl]-oxime (Compound 17)
1H-indole-3-carbaldehyde O—[6-morpholin-4-yl-2-(2-pyridin-2-yl-ethoxy)-pyrimidin-4-yl]-oxime (Compound 18)
N-(1H-indol-3-ylmethylene)-N′-{6-morpholin-4-yl-2-[2-(pyridin-3-yloxy)-ethoxy]-pyrimidin-4-yl}-hydrazine (Compound 19)
N-(3-methyl-benzylidene)-N′-{6-morpholin-4-yl-2-[2-(pyridin-3-yloxy)-ethoxy]-pyrimidin-4-yl}-hydrazine (Compound 20)
butyl-{4-[N′-(1H-indol-3-ylmethylene)-hydrazino]-6-morpholin-4-yl-pyrimidin-2-yl}-amine (Compound 21)
N-(3-methyl-benzylidene)-N′-[6-morpholin-4-yl-2-(pyridin-3-yloxy)-pyrimidin-4-yl]-hydrazine (Compound 22)
N-(3-methylbenzlidene)-N′-(5-methyl-6-morpholin-4-yl-2-phenylpyrimidin-4-yl)hydrazine (Compound 23)
N-(3-methyl-benzylidene)-N′-(2-phenyl-6-thiomorpholin-4-yl-pyrimidin-4-yl)-hydrazine (Compound 24)
(2,3-dimethyl-1H-indole-5-yl)-(6-morpholin-4-yl-2-[2-(pyridin-3-yloxy)-ethoxy]-pyrimidin-4-yl)-amine (Compound 25)
(2,3-dimethyl-1H-indole-5-yl)-{4-morpholin-4-yl-6-[2-(pyridin-3-yloxy)-ethoxy]-pyrimidin-2-yl}-amine (Compound 26)
3-{4-[N′-(3-methyl-benzylidene)-hydrazino]-6-morpholin-4-yl-pyrimidin-2-yl}-propionic acid ethyl ester (Compound 27)
N-(3-methyl-benzylidene)-N′-{6-morpholin-4-yl-2-[2-(1-oxy-pyridin-2-yl)-ethoxy]-pyrimidin-4-yl}-hydrazine (Compound 28)
1-(2-{4-[N′-(3-methyl-benzylidene)-hydrazino]-6-morpholin-4-yl-pyrimidin-2-yloxy}-ethyl)-1H-pyridin-2-one (Compound 29)
N-(3-iodo-benzylidene)-N′-[6-morpholin-4-yl-2-(2-pyridin-2-yl-ethoxy)-pyrimidin-4-yl]-hydrazine (Compound 30)
N-(3-fluoro-benzylidene)-N′-[6-morpholin-4-yl-2-(2-pyridin-2-yl-ethoxy)-pyrimidin-4-yl]-hydrazine (Compound 31)
N-(3-chloro-benzylidene)-N′-[6-morpholin-4-yl-2-(2-pyridin-2-yl-ethoxy)-pyrimidin-4-yl]-hydrazine (Compound 32)
N-(3-bromo-benzylidene)-N′-[6-morpholin-4-yl-2-(2-pyridin-2-yl-ethoxy)-pyrimidin-4-yl]-hydrazine (Compound 33)
3-{[6-morpholin-4-yl-2-(2-pyridin-2-yl-ethoxy)-pyrimidin-4-yl]-hydrazonomethyl}-benzoic acid methyl ester (Compound 34)
1-(2-{4-[N′-(3-iodo-benzylidene)-hydrazino]-6-morpholin-4-yl-pyrimidin-2-yloxy}-ethyl)-1H-pyridin-2-one (Compound 35)
3-{[6-morpholin-4-yl-2-(2-pyridin-2-yl-ethoxy)-pyrimidin-4-yl]-hydrazonomethyl}-benzoic acid N-methyl amide (Compound 36)
(3-{[6-morpholin-4-yl-2-(2-pyridin-2-yl-ethoxy)-pyrimidin-4-yl]-hydrazonomethyl}-phenyl)-methanol (Compound 37)
N,N-Diethyl-4-{4-[N′-(3-methyl-benzylidene)-hydrazino]-6-morpholin-4-yl-pyrimidin-2-yl}-butyramide (Compound 38)
4-{4-[N′-(3-Methyl-benzylidene)-hydrazino]-6-morpholin-4-yl-pyrimidin-2-yl}-1-(4-methyl-piperazin-1-yl)-butan-1-one (Compound 39)
4-{4-[N′-(3-Methyl-benzylidene)-hydrazino]-6-morpholin-4-yl-pyrimidin-2-yl}-N-pyridin-4-ylmethyl-butyramide (Compound 40)
4-{4-[N′-(3-Methyl-benzylidene)-hydrazino]-6-morpholin-4-yl-pyrimidin-2-yl}-N-pyridin-4-yl-butyramide (Compound 41)
2-{4-[N′-(3-Methyl-benzylidene)-hydrazino]-6-morpholin-4-yl-pyrimidin-2-yloxy}-1-pyridin-2-yl-ethanol (Compound 42)
6-(2-{4-[N′-(3-Methyl-benzylidene)-hydrazino]-6-morpholin-4-yl-pyrimidin-2-yloxy}-ethyl)-pyridin-3-ol (Compound 43)
6-(2-{4-[N′-(3-Hydroxymethyl-benzylidene)-hydrazino]-6-morpholin-4-yl-pyrimidin-2-yloxy}-ethyl)-pyridin-3-ol (Compound 44)

The structures of these compounds are depicted below:

### Compound 1:

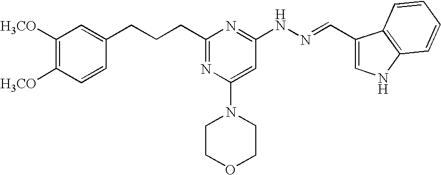

### Compound 2:

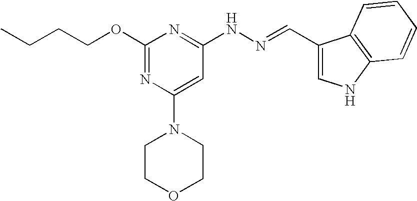

### Compound 3:

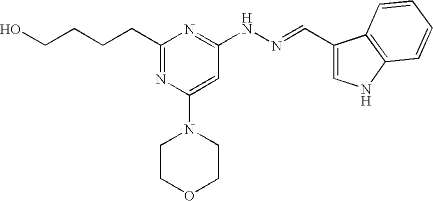

### Compound 4:

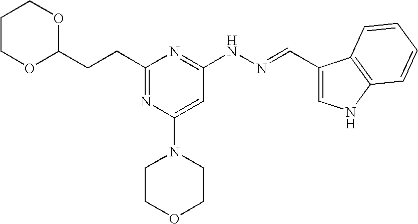

### Compound 5:

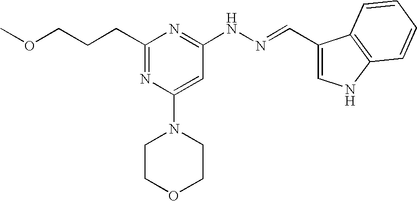

### Compound 6:

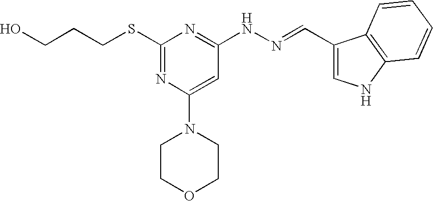

### Compound 7:

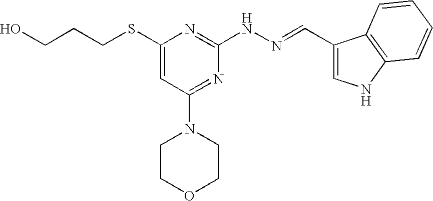

### Compound 8:

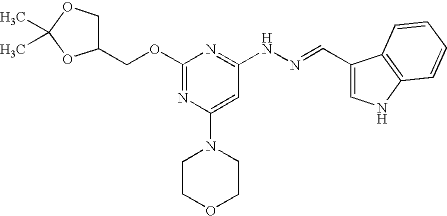

### Compound 9:

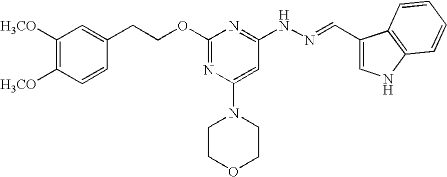

### Compound 10:

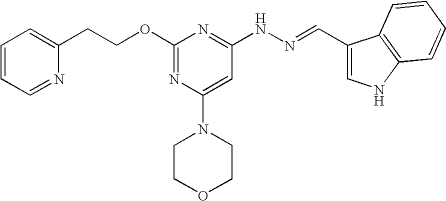

### Compound 11,

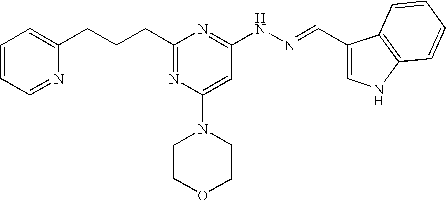

### Compound 12:

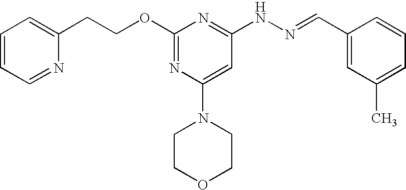

### Compound 13:

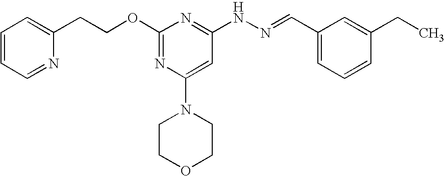

### Compound 14:

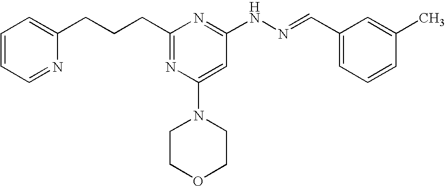

### Compound 15:

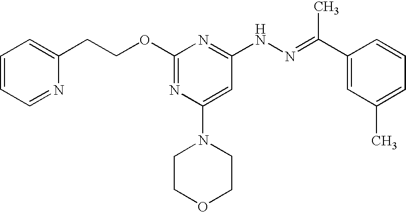

### Compound 16:

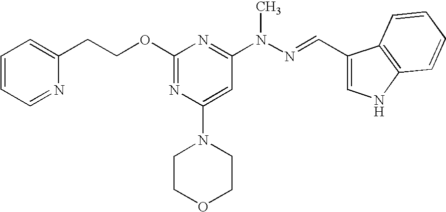

### Compound 17:

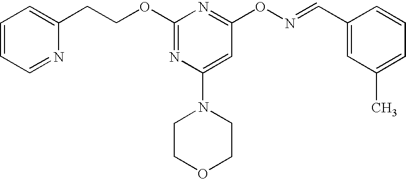

### Compound 18:

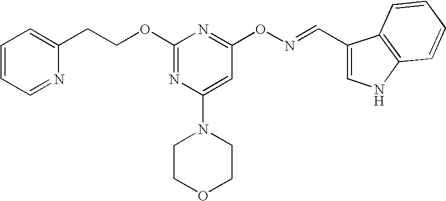

### Compound 19:

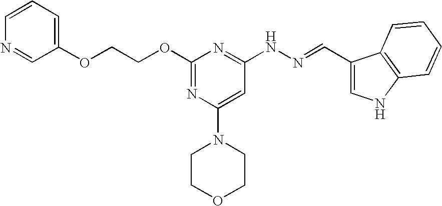

### Compound 20:

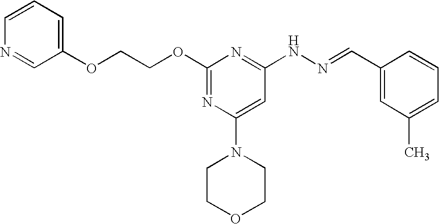

### Compound 21:

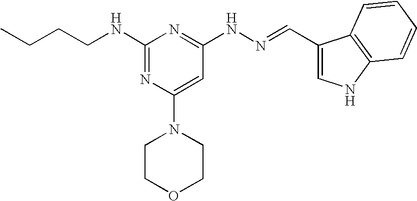

### Compound 22:

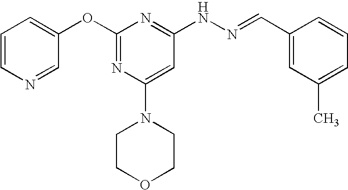

### Compound 23:

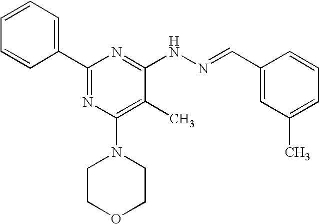

### Compound 24:

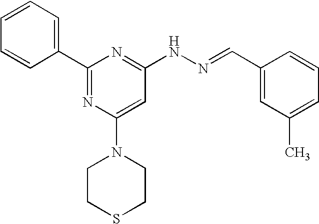

### Compound 25:

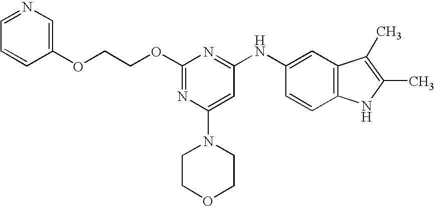

### Compound 26:

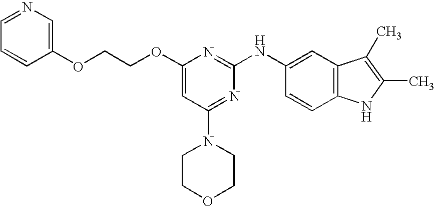

### Compound 27:

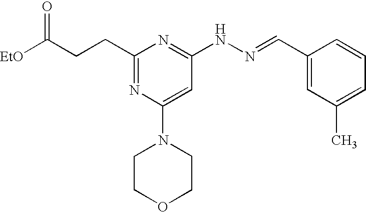

### Compound 28:

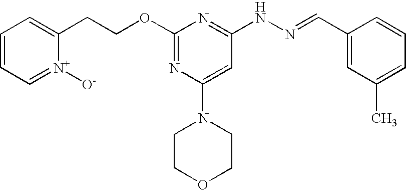

### Compound 29:

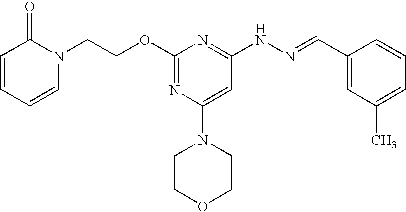

### Compound 30:

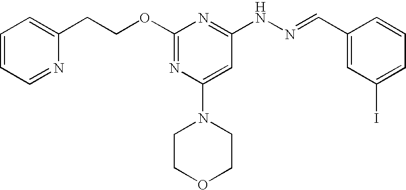

### Compound 31:

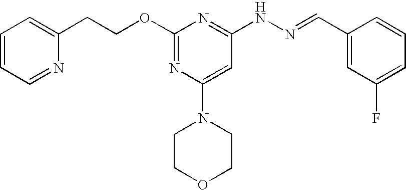

### Compound 32:

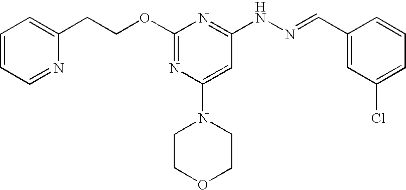

### Compound 33:

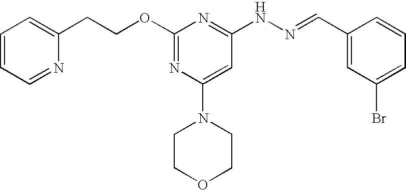

### Compound 34:

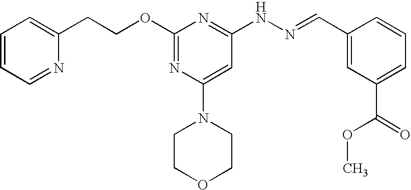

### Compound 35:

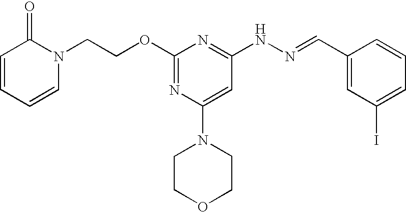

### Compound 36:

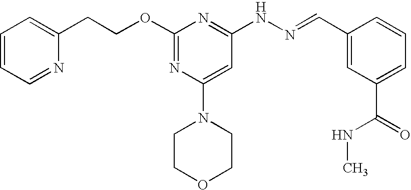

### Compound 37:

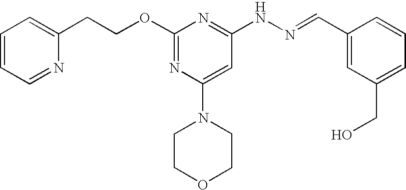

### Compound 38:

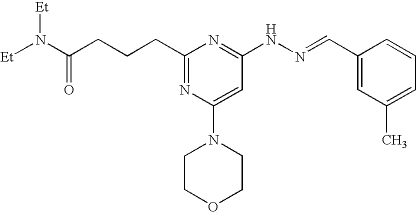

### Compound 39:

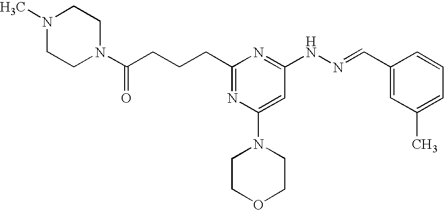

### Compound 40:

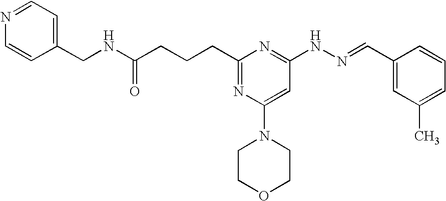

### Compound 41:

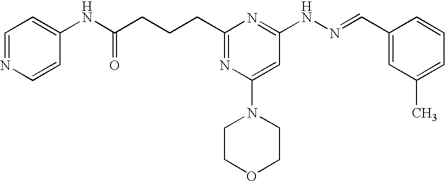

### Compound 42:

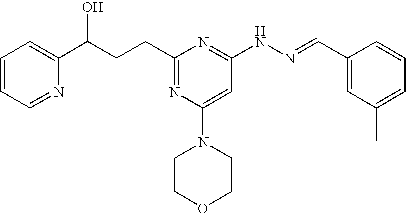

### Compound 43:

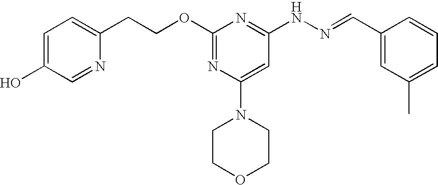

### Compound 44:

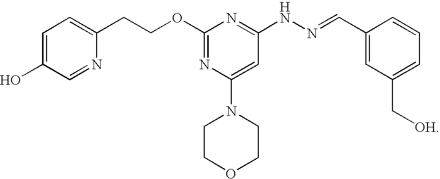

Referring to formula (I′), a subset of the triazine compounds of this invention is featured by that R¹ is NC(R^{a}R^{b}). In these compounds, W can be O; R₅ can be H or alkyl; X can be NR^{c}; R^{c} can be H, methyl, ethyl, or acetyl; Y can be O or CH₂, and n can be 0, 1, 2, 3, or 4. In some embodiments, R₃ is aryl, heteroaryl (e.g., pyridinyl), OR^{c}, SR^{c}, C(O)OR^{c}, or C(O)NR^{c}R^{d}. In other embodiments, R₃ is

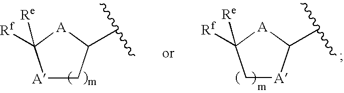

in which each of A and A′, independently, is O, S, or NH; each of R^{e} and R^{f}, independently, is H, alkyl, aryl, or heteroaryl; and m is 1 or 2.

In this subset of triazine compounds, R^{a} or R^{b}, preferably, is

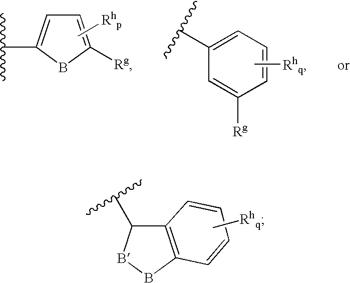

in which B is NR^{f}, O, or S; B′ is N or CR^{f}; R^{g} is H, alkyl, or alkoxyl; R^{h} is halogen, CN, hydroxyl, alkyl, aryl, heteroaryl, alkoxyl, aryloxyl, or heteroaryloxyl; Rⁱ is H, alkyl, or alkylcarbonyl; p is 0, 1, or 2; and q is 0, 1, 2, 3, or 4. Preferably, B is NRⁱ; B′ is CH; R^{g} is H, methyl, ethyl, methoxy, or ethoxy; R^{h} is F, Cl, CN, methoxy, methyl, or ethoxy; Rⁱ is H, methyl, ethyl, or acetyl; and q is 0, 1, or 2.

Another subset of the triazine compounds of this invention is featured by that R¹ is aryl or heteroaryl. In these compounds, W can be O; R₅ can be H or alkyl; X can be NR^{c}; R^{c} can be H, methyl, ethyl, or acetyl; Y can be Q or CH₂, and n can be 0, 1, 2, 3, or 4. In some embodiments, R₃ is aryl, heteroaryl (e.g., pyridinyl), OR^{c}, SR^{c}, C(O)OR^{c}, or C(O)NR^{c}R^{d}. In other embodiments, R₃ is

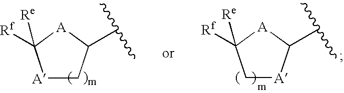

in which each of A and A′, independently, is O, S, or NH; each of R^{e} and R^{f}, independently, is H, alkyl, aryl, or heteroaryl; and m is 1 or 2.

In this second subset of triazine compounds, R₁, preferably, is

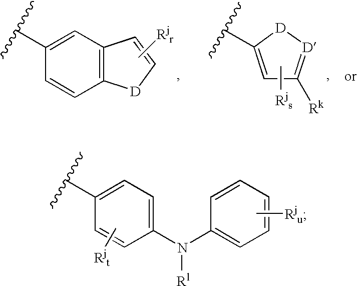

in which D is O, S, or NR^{m}; D′ is N or CR^{m}; R^{j} is halogens CN, hydroxyl, alkyl, aryl, heteroaryl, alkoxyl, aryloxyl, or heteroaryloxyl; R^{k} is aryl or hetereoaryl; R^{l} is H, alkyl, or alkylcarbonyl; R^{m} is H, alkyl, or alkylcarbonyl; r is 0, 1, or 2; s is 0 or 1; t is 0, 1, 2, 3, or 4; and u is 0, 1, 2, 3, 4, or 5. Preferably, R₁ is

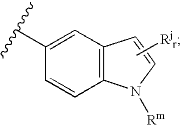

and R^{j} is methyl, ethyl, propyl, or benzyl; and r can be 1 or 2.

In another aspect, this invention also features triazine compounds of formula (I′), wherein R₁ is NC(R^{a}R^{b}), aryl, or heteroaryl; each of R₂, R₄, and R₅, independently, is R^{c}, halogen, nitro, nitroso, cyano, azide, isothionitro, SR^{c}, or OR^{c}; R₃ is R^{c}, alkenyl, alkynyl, aryl, heteroaryl, cyclyl, heterocyclyl, OR^{c}, OC(O)R^{c}, SO₂R^{c}, S(O)R^{c}, S(O₂)NR^{c}R^{d}, SRO, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d}; n is 0, 1, 2, 3, 4, 5, 6, or 7; X is O, S, S(O), S(O₂), or NR^{c}; Y is a covalent bond, CH₂, C(O), C═N—R^{c}, C═N—OR^{c}, C═N—SR^{c}, O, S, S(O), S(O₂), or NR^{c}; Z is CH; and W is O, S, S(O), S(O₂), NR^{c}, or NC(O)R^{c}; in which each of R^{a} and R^{b}, independently, is H, alkyl, aryl, heteroaryl; and each of R^{c} and R^{d}, independently, is H, alkyl, or alkylcarbonyl. A subset of the triazine compounds is featured by that R¹ is NC(R^{a}R^{b}); and another subset is featured by that R¹ is aryl or heteroaryl.

Set forth below are exemplary compounds (Compounds 100-116) useful in this invention:

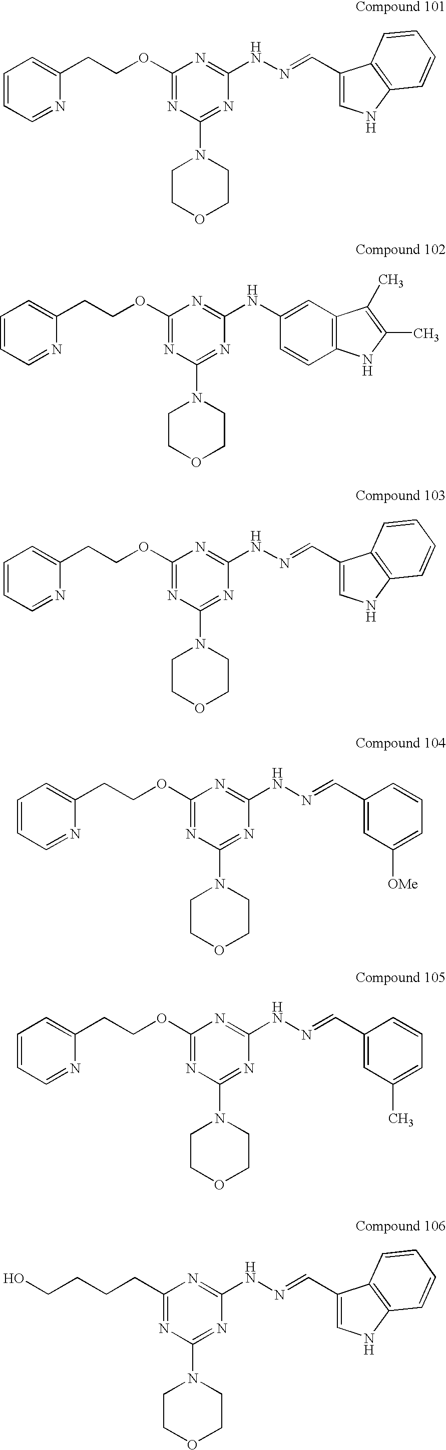

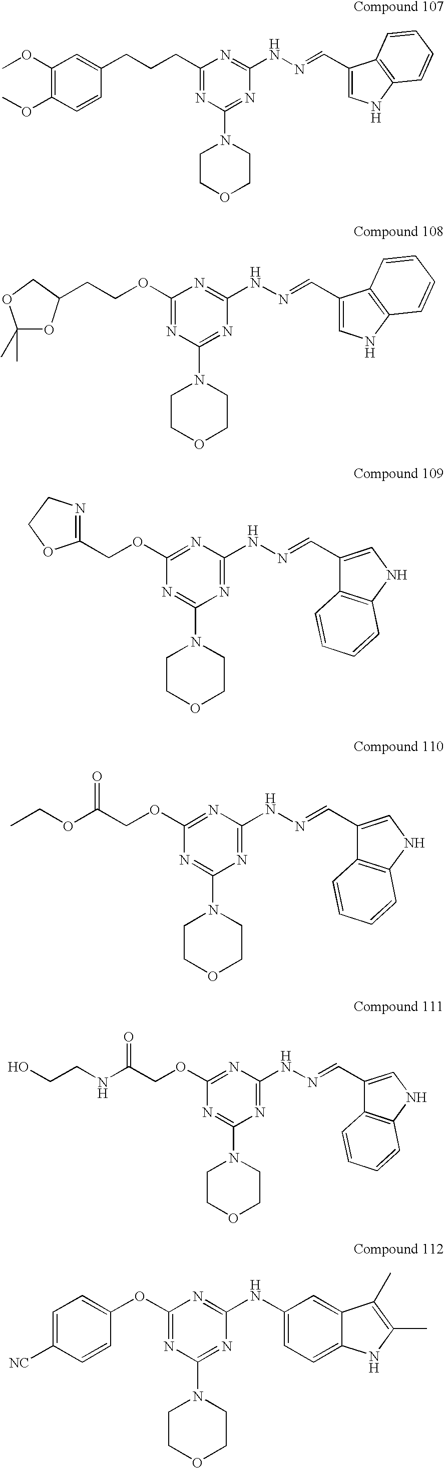

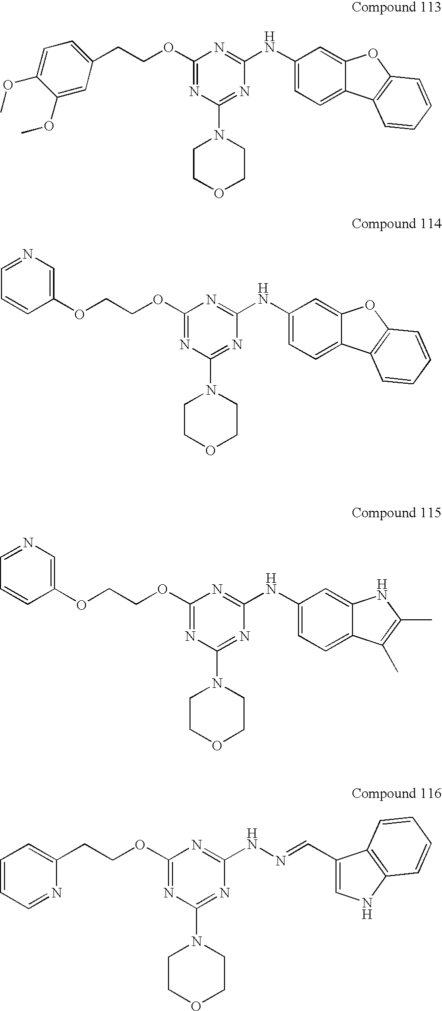

Referring to formula (I″), a subset of these compounds is featured by that A is NR^{e}, and B is N. Another subset of the compounds are those wherein Z is N and W is O; or X is NR^{e}.

Yet another subset of the compounds are those wherein each of U and V is N. In these compounds, A can be NR^{e}, B can be N, Y can be NR^{e} or O, Z can be N, W can be O, R₁ can be aryl, and R₃ can be halogen, CN, alkyl, aryl, hetereoaryl, OR^{a}, OC(O)R^{a}, NR^{a}NR^{b}, NR^{a}C(O)R^{b}, C(O)OR^{a}, or C(O)NR^{a}R^{b}. In some embodiments, R₃ is aryl, hetereoaryl (e.g., pyridinyl, triazolyl, tetrazolyl, pyrimidinyl, thiazolyl, indolyl, or indolizinyl), aryloxyl, or hetereoaryloxyl. In some other embodiments, R₁ is

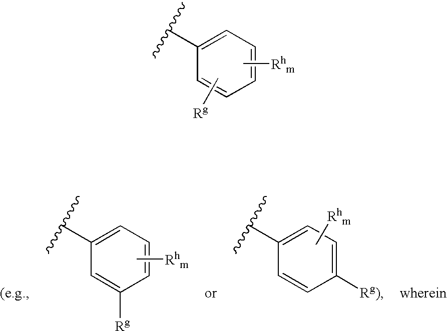

R⁹ is H, halogen, CN, alkyl, or alkoxyl; R^{h} is halogen (F, Cl, Br; or I), CN, hydroxyl, amino, alkyl (e.g., Me, Et, Pr, or i-Pr), aryl, heteroaryl, alkoxyl (e.g., OMe or OEt), aryloxyl, heteroaryloxyl, acyl (e.g., C(O)CH₃), alkoxycarbonyl (e.g., —C(O)OCH₃), alkylcarbonoxyl (e.g., OC(O)CH₃), mono- or dialkylaminocarbonyl (e.g., NC(O(CH₃)₂)), amidinyl (e.g., C(NH)NH₂), ureayl (e.g., NHC(O)NH₂), guanadinyl (e.g., NHC(NH)NH₂), sulfonyl (e.g., SO₂CH₃), or sulfonamidyl (e.g., SO₂NH₂); and m is 0, 1, 2, 3, or 4.

Set forth below are exemplary compounds useful in this invention:

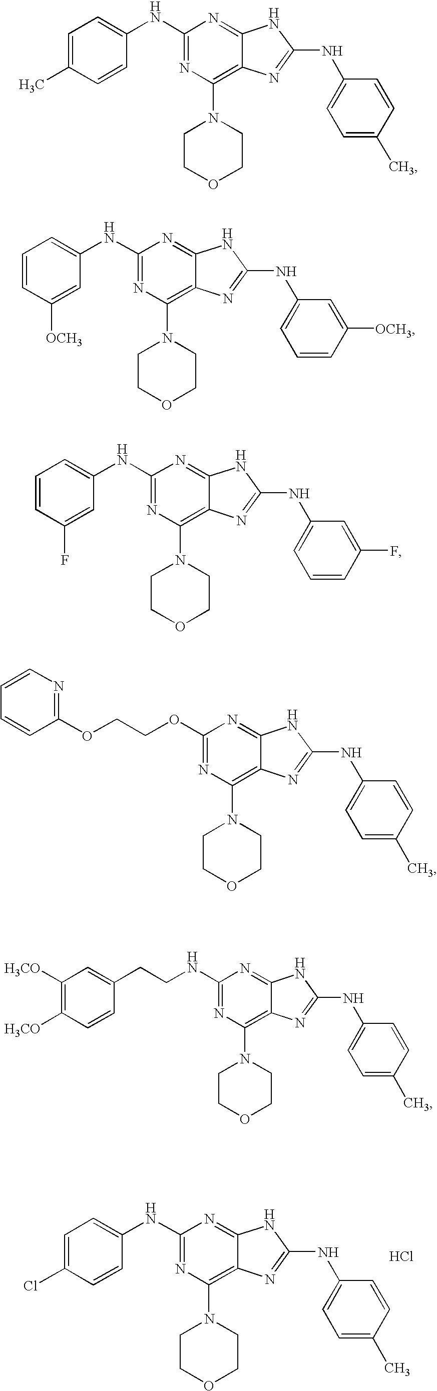

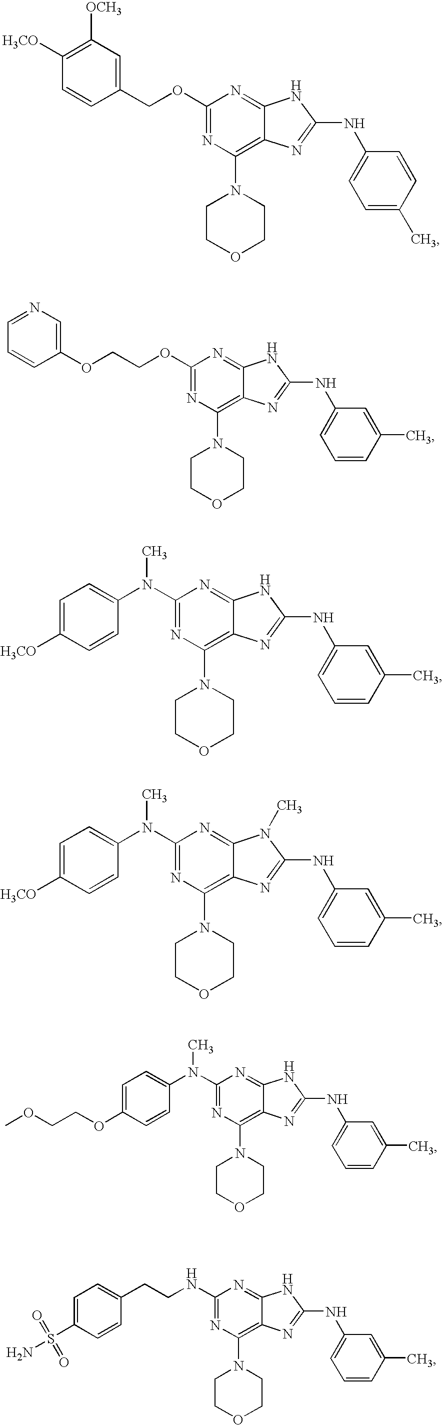

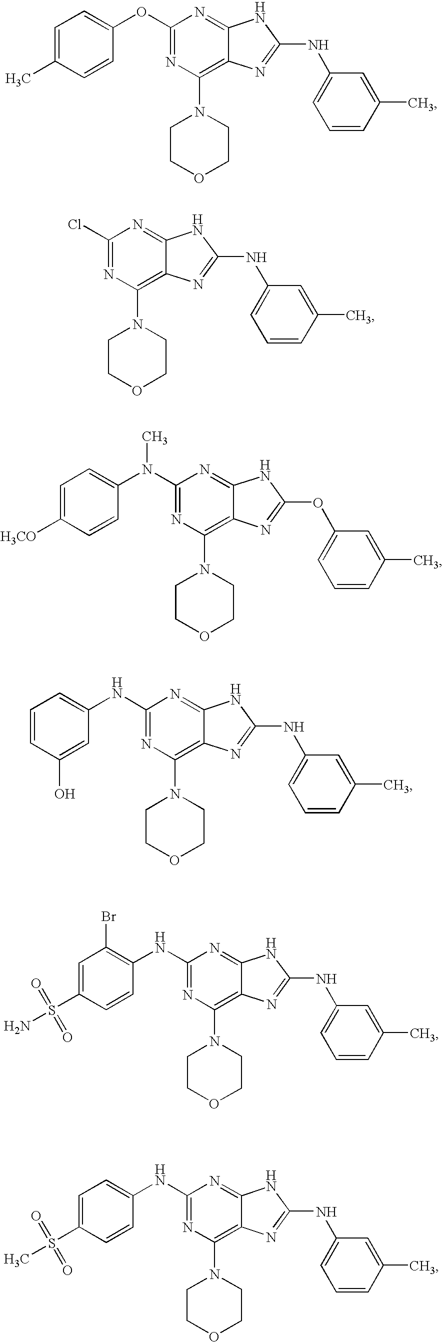

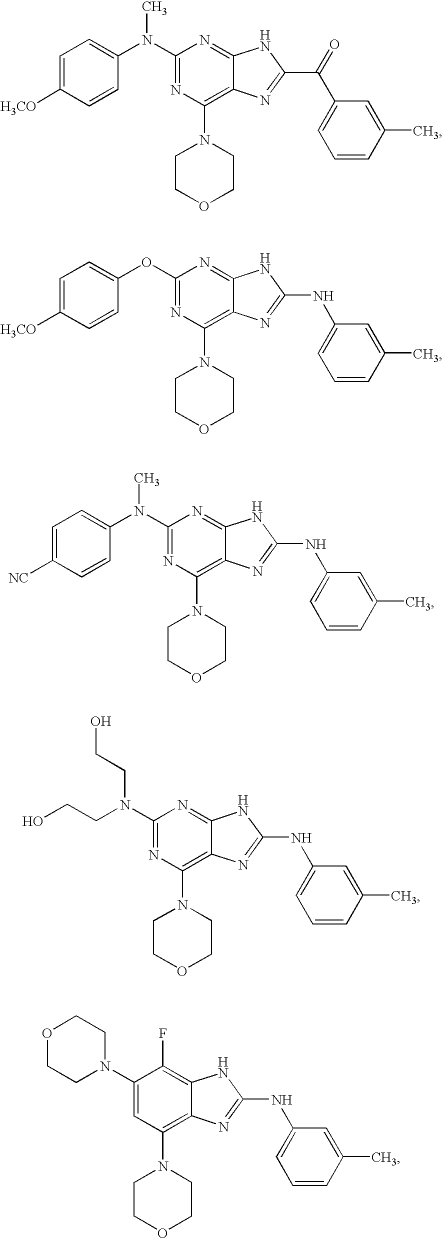

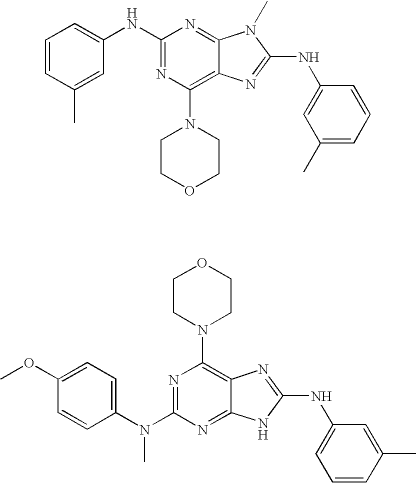

Other exemplary compounds useful in this invention are described in commonly owned co-pending U.S. patent application Ser. Nos. 10/000,742, 10/192,347 and 10/305,039 and PCT International patent application serial number PCT/US02/38161; in commonly owned U.S. Pat. No. 6,384,032 and co-pending U.S. patent application Ser. No. 10/006,624; and in commonly owned co-pending U.S. patent application Ser. No. 60/418,984 (the disclosures of which are hereby incorporated by reference in their entirety).

Methods of Treatment and Prevention

In accordance with the invention, an effective amount of a compound of formula (I), formula (I′), or formula (I″) or a pharmaceutically acceptable salt, solvate, clathrate, and prodrug thereof, or a pharmaceutical composition comprising a compound of formula (I), formula (I′), or formula (I″) or a pharmaceutically acceptable salt, solvate, clathrate, and prodrug thereof, is administered to an patient in need of treatment or prevention of a disorder associated with excessive bone loss (including, without limitation, periodontal disease, osteoporosis, estrogen deficiency, Paget's disease, inflammatory bone loss, bone malignancy, hyperparathyroidism, arthritis, and osteoporosis). Other conditions, diseases and disorders that would benefit from such uses are known to those of skill in the art.

Responsiveness of a particular condition, disease or disorder to compounds and compositions of this invention can be measured directly by comparison against conventional drugs, or can be inferred based on an understanding of disease etiology and progression. There are a number of cellular and bone resorption assay systems that are widely accepted in the art as predictive of in vivo effects. As the bone resorption assay uses material that includes all bone cells, it is an ex vivo assay. Thus, the showing that a compound of this invention inhibits bone resorption in these assays is evidence of the clinical utility of these for treating or preventing conditions associated with excessive bone loss. Various scientific publications (such as Carano et al. J. Clin. Invest. 85: 456-461 (1990); Blair & Schlesinger, The Biology and Physiology of the Osteoclast, CRC Press, Eds., Gay, C. V. and Rifkin, B. R., pp. 259-288 (1992); and Vaananen et al., J. Cell Biology 111: 1305-1311 (1990)) support the fact that such assays are accepted as being predictive of in-vivo activity. Furthermore, the in vitro effects of Herbimycin A on bone resorption were shown to correlate with in vivo activity (Yoneda et al., J. Clin. Invest. 91: 2791-95 (1993)).

In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder or symptoms thereof.

In certain embodiments, the compounds of the invention or the compositions of the invention are administered to a patient, preferably a human, as a prophylactic or preventative measure against particular conditions, diseases and disorders. As used herein, “prevention” or “preventing” refers to a reduction of the risk of acquiring a given condition, disease or disorder. In a preferred mode of the embodiment, the compositions of the present invention are administered as a preventative measure to a patient, preferably a human, having a genetic predisposition to any of the disorders described herein. In each of the therapeutic or prophylactic methods of the invention, a therapeutically or prophylactically effective amount of a compound of formula (I), formula (I′), or formula (I″) or a pharmaceutically acceptable salt, solvate, clathrate, and prodrug thereof is administered to a patient.

The compounds of formula (I), formula (I′), or formula (I″) or pharmaceutically acceptable salts, solvates, clathrates, and prodrugs thereof can be assayed in vitro or in vivo, for the desired therapeutic or prophylactic activity, prior to use in humans. For example, animal model systems can be used to demonstrate the safety and efficacy of compounds of this invention.

Without wishing to be bound by theory, it is believed that the compounds and compositions of this invention inhibit osteoclast formation and as a result, may be used to treat or prevent disorders associated with excessive bone loss. It should be noted, however, that the compounds might act by a secondary or a different activity, such as, without limitation, inhibiting resorptive osteoclast activity, increasing production of parathyroid hormone, enhancing osteoblast activity and/or otherwise increasing bone mass. It should also be noted that various compounds of formula (I), formula (I′), or formula (I″) and similar structures have been described in commonly owned co-pending U.S. patent application Ser. Nos. 10/000,742, 10/192,347, 10/305,039 and PCT International patent application serial number PCT/US02/38161 (the disclosures of which are hereby incorporated by reference in their entirety). To the extent not set forth in this application, the compounds described those prior filings are hereby included in the definition of formula (I), (I′), and (I″). The compounds of those prior filings were described as inhibiting production of IL-12. Without wishing to be bound to theory, we were surprised to discover that such compounds can inhibit osteoclast formation and prevent or treat disorders associated with excessive bone loss. Published research suggests that IL-12 itself (both alone and in synergy with IL-18) inhibits osteoclast formation (Horwood et al., J Immun. 1663(8), 4915-21, 2001). Accordingly, one would not expect an IL-12 inhibitor to also inhibit osteoclast formation. However, as demonstrated in the examples that follow, the compounds of this invention possess this activity.

Pharmaceutical Compositions and Dosage Forms

Pharmaceutical compositions and dosage forms of the invention comprise one or more active ingredients in relative amounts and formulated in such a way that a given pharmaceutical composition or dosage form inhibits the uptake of calcium. Preferred pharmaceutical compositions and dosage forms comprise a compound of formula (I), (I′), or (I″), or a pharmaceutically acceptable prodrug, salt, solvate, or clathrate thereof, optionally in combination with one or more additional active agents.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intra arterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Eastern Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., pp. 379-80 (1995). In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a compound of formula (I), (I′), or (I″), or a pharmaceutically acceptable salt, solvate, clathrate, or prodrug thereof in an amount of from about 0.1 mg to about 1000 mg, preferably in an amount of from about 1 mg to about 500 mg, and most preferably in an amount of from about 5 mg to about 250 mg. The typical total daily dosage of the compound of formula (I), (I′), or (I″), or a pharmaceutically acceptable salt, solvate, clathrate, or prodrug thereof can range from about 0.1 mg to about 5000 mg per day, preferably. In an amount from about 1 mg to about 1000 mg per day, more preferably from about 10 mg to about 500 mg per day. It is within the skill of the art to determine the appropriate dose and dosage form for a given patient.

Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Controlled Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to Initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of formula (I), (I′), or (I″), or a pharmaceutically acceptable salt, solvate, hydrate, clathrate, or prodrug thereof, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of formula (I), (I′), or (I″) by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.

Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active Ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

Kits

This invention encompasses kits which, when used by the medical practitioner, can simplify the administration of appropriate amounts of active ingredients to a patient.

A typical kit of the invention comprises a unit dosage form of an effective amount of a compound of formula (I), (I′), or (I″), or a pharmaceutically acceptable prodrug, salt, solvate, hydrate, or clathrate thereof, and a device that can be used to administer the active ingredient. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles for such use include, but are not limited to: Water for Injection USP; aqueous vehicles such as, but not limited to, Sodium Chloride Injection, Ringer'Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Combination Therapy

The methods for treating or preventing disorders associated with excessive bone loss in a patient in need thereof can further comprise administering to the patient being administered a compound of this invention, an effective amount of one or more other therapeutic agents. Such therapeutic agents may include other therapeutic agents such as those conventionally used to prevent or treat disorders associated with excessive bone resorption or symptoms thereof. For example, such other agents include anti-resorptive agents for example progestins, polyphosphonates, bisphosphonate(s), estrogen agonists/antagonists, estrogen (such as Premarin®), estrogen/progestin combinations, and estrogen derivatives (such as estrone, estriol or 17α,17β-ethynyl estradiol).

In such combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the ether therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, dthynodiol diacetate, etonogestrel, fluorogestone acetate, gestaclone, gestodene, gestonorone caproate, gestrinone, haloprogesterone, hydroxyprogesterone, caproate, levonorgestrel, lynestrenol, medrogestone, medroxyprogesterone acetate, melengestrol acetate, methynodiol diacetate, norethindrone, norethindrone acetate, norethynodrel, norgestimate, norgestomet, norgestrel, oxogestone phenpropionate, progesterone, quingestanol acetate, quingestrone, and tigestol. Preferred progestins are medroxyprogestrone, norethindrone and norethynodrel.

Exemplary bone resorption inhibiting polyphosphonates include polyphosphonates of the type disclosed in U.S. Pat. No. 3,683,080. Preferred polyphosphonates are geminal dipolyphosphonates (also referred to as bis-phosphonates). Tiludronate disodium is an especially preferred polyphosphonate. Ibandronic acid is an especially preferred polyphosphonate. Alendronate is an especially preferred polyphosphonate. Zoledronic acid is an especially preferred polyphosphonate. Other preferred polyphosphonates are 6-amino-1-hydroxy-hexylidene-biphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl-3-amino-1-hydroxy-1,1-diphosphonic acid, phenyl amino methane diphosphonic acid, N,N-dimethylamino methane diphosphonic acid, N(2-hydroxyethyl)amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used for this purpose. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue; and blocking the actions of estrogen in one or more tissues. Such activities are readily determined by those skilled in the art of standard assays including estrogen receptor binding assays, standard bone histomorphometric and densitometer methods, and E. F Eriksen et al., Bone Histomorphometry, Raven Press, New York, pp. 1-74 (1994); S. J. Grier et. al., The Use of Dual-Energy X-Ray Absorptiometry in Animals, Inv. Radiol. 31(1): 50-62 (1996); Wahner H. W. and Fogelman I., The Evaluation of Osteoporosis: Dual Energy X-Ray Absorptiometry in Clinical Practice, Martin Dunitz Ltd., London, pp. 1-296 (1994)). A variety of these compounds are described and referenced below.

A preferred estrogen agonist/antagonist is droloxifene: (phenol, 3-(1-(4-(2-(dimethylamino)ethoxy)phenyl)-2-phenyl-1-butenyl)-, (E)-) and related compounds which are disclosed in U.S. Pat. No. 5,047,431. Another preferred estrogen agonist/antagonist is 3-(4-(1,2-diphenyl-but-1-enyl)-phenyl)-acrylic acid, which is disclosed in Wilson et al., Endocrinology 138: 3901-11 (1997). Another preferred estrogen agonist/antagonist is tamoxifen: (ethanamine, 2-(−4-(1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516. Another related compound is 4-hydroxy tamoxifen which is disclosed in U.S. Pat. No. 4,623,660.

A preferred estrogen agonist/antagonist is raloxifene: (methanone, (6-hydroxy-2-(4-hydroxyphenyl)benzo[b]thien-3-yl)(4-(2-(1-piperidinyl)ethoxy)phenyl)hydrochloride) which is disclosed in U.S. Pat. No. 4,418,068. Another preferred estrogen agonist/antagonist is toremifene: (ethanamine, 2-(4-(4-chloro-1,2-diphenyl-1-butenyl)phenoxy)-N,N-dimethyl-, (Z)-, 2-hydroxy-1,2,3-propanetricarboxylate (1:1) which is disclosed in U.S. Pat. No. 4,996,225. Another preferred estrogen agonist/antagonist is centchroman: 1-(2-((4-(-methoxy-2,2,dimethyl-3-phenyl-chroman-4-yl)-phenoxy)-ethyl)-pyrrolidine, which is disclosed in U.S. Pat. No. 3,822,287. Also preferred is levormeloxifene. Another preferred estrogen agonist/antagonist is idoxifene: (E)-1-(2-(4-(1-(4-iodo-phenyl)-2-phenyl-but-1-enyl)-phenoxy)-ethyl)-pyrrolidinone, which is disclosed in U.S. Pat. No. 4,839,155. Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058. Another preferred estrogen agonist/antagonist is 6-(4-hydroxy-phenyl)-5-(4-(2-piperidin-1-yl-ethoxy)-benzyl)-naphthalen-2-ol which is disclosed in U.S. Pat. No. 5,484,795. Another preferred estrogen agonist/antagonist is (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer Inc. Other preferred estrogen agonist/antagonists include compounds as described in U.S. Pat. No. 5,552,412. Especially preferred compounds described therein are: cis-6-(4-fluoro-phenyl)-5-(4-(2-piperidin-1-yl-ethoxy)-phenyl)-5,6,7,8-tetrahydro-naphthalene-2-ol; (−)-cis-6-phenyl-5-(4-(2-pyrrolidin-1-yl-ethoxy)-phenyl)-5,6,7,8-tetrahydro-naphthalene-2-ol; cis-6-phenyl-5-(4-(2-pyrrolidin-1-yl-ethoxy)-phenyl)-5,6,7,8-tetrahydro-naphthalene-2-ol; cis-1-(6′-pyrrolodinoethoxy-3′-pyridyl)-2-phenyl-6-hydroxy-1,2,3,4-tetrahydronaphthalene; 1-(4′-pyrrolidinoethoxyphenyl)-2-(4″fluorophenyl)-6-hydroxy-1,2,3,4-tetrahydroisoquinoline; cis-6-(4-hydroxyphenyl)-5-(4-(2-piperidin-1-yl-ethoxy)-phenyl)-5,6,7,8-tetrahydro-naphthalene-2-ol; and 1-(4′-pyrrolidinolethoxyphenyl)-2-phenyl-6-hydroxy-1,2,3,4-tetrahydroisoquinoline. Other estrogen agonist/antagonists are described in U.S. Pat. No. 4,133,814. U.S. Pat. No. 4,133,814 discloses derivatives of 2-phenyl-3-aroyl-benzothiophene and 2-phenyl-3-aroylbenzothiophene-1-oxide.

Those skilled in the art will recognize that other bone anabolic agents, also referred to as bone mass augmenting agents, may be used in conjunction with the compounds of this invention. A bone mass augmenting agent is a compound that augments bone mass to a level which is above the bone fracture threshold as detailed in the World Health Organization Study World Health Organization, “Assessment of Fracture Risk and its Application to Screening for Postmenopausal Osteoporosis (1994). Report of a WHO Study Group. World Health Organization Technical Series 843.” Any prostaglandin, or prostaglandin agonist/antagonist may be used in combination with the compounds of this invention. Those skilled in the art will recognize that IGF-1, sodium fluoride, parathyroid hormone (PTH), active fragments of parathyroid hormone, growth hormone or growth hormone secretagogues may also be used. The following paragraphs describes in greater detail exemplary compounds that may be administered in combination with compounds of this invention

Prostaglandins: The term prostaglandin refers to compounds which are analogs of the natural prostaglandins PGD₁, PGD₂, PGE₂, PGE₁ and PGF₂ which are useful in the treatment of osteoporosis and other disorders associated with excessive osteoclastic bone resorption. These compounds bind to the prostaglandins receptors. Such binding is readily determined by those skilled in the art of standard assays (e.g., S. An et al., Cloning and Expression of the EP₂ Subtype of Human Receptors for Prostaglandin E₂ Biochemical and Biophysical Research Communications, 197(1): 263-270 (1993)).

Prostaglandins are alicyclic compounds related to the basic compound prostanoic acid. The carbon atoms of the basic prostaglandin are numbered sequentially from the carboxylic carbon atom through the cyclopentyl ring to the terminal carbon atom on the adjacent side chain. Normally the adjacent side chains are in the trans orientation. The presence of an oxo group at C-9 of the cyclopentyl moiety is indicative of a prostaglandin within the E class while PGE₂ contains a trans unsaturated double bond at the C₁₃-C₁₄ and a cis double bond at the C₅-C₆ position.

A variety of prostaglandins are described and referenced below. However, other prostaglandins will be known to those skilled in the art. Exemplary prostaglandins are disclosed in U.S. Pat. Nos. 4,171,331 and 3,927,197, Norrdin et al., The Role of Prostaglandins in Bone in Vivo, Prostaglandins Leukotriene Essential Fatty Acids 41: 139-150 (1990) is a review of bone anabolic prostaglandins. Any prostaglandin agonist/antagonist may be used in combination with the compounds of this invention. The term prostaglandin agonist/antagonist refers to compounds which bind to prostaglandin receptors (eg., An S. et al., Cloning and Expression of the EP₂ Subtype of Human Receptors for Prostaglandin E₂, Biochemical and Biophysical Research Communications 197(1): 263-70 (1993)) and mimic the action of prostaglandin in vivo (e.g., stimulate bone formation and increase bone mass). Such actions are readily determined by those skilled in the art of standard assays. Eriksen E. F. et al., Bone Histomorphometry, Raven Press, New York, 1994, pp. 1-74; S. J. Grier et al., The Use of Dual-Energy X-Ray Absorptiometry In Animals, Inv. Radiol. 31(1): 50-62 (1996); H. W. Wahner and I. Fogelman, The Evaluation of Osteoporosis: Dual Energy X-Ray Absorptiometry in Clinical Practice, Martin Dunitz Ltd. London, pp. 1-296 (1994). A number of these compounds are described and reference below. However, other prostaglandin agonists/antagonists will be known to those skilled in the art. Exemplary prostaglandin agonists/antagonists are disclosed as follows. U.S. Pat. No. 3,932,389 discloses 2-descarboxy-2-(tetrazol-5-yl)-11-desoxy-15-substituted-omega-pentanorprostaglandins useful for bone formation activity. U.S. Pat. No. 4,018,892, discloses 16-aryl-13,14-dihydro-PGE₂ p-biphenyl esters useful for bone formation activity. U.S. Pat. No. 4,219,483, discloses 2,3,6-substituted-4-pyrones useful for bone formation activity. U.S. Pat. No. 4,132,847, discloses 2,3,6-substituted-4-pyrones useful for bone formation activity. U.S. Pat. No. 4,000,309, discloses 16-aryl-13,14-dihydro-PGE₂ p-biphenyl esters useful for bone formation activity. U.S. Pat. No. 3,982,016, discloses 16-aryl-13,14-dihydro-PGE₂ p-biphenyl esters useful for bone formation activity. U.S. Pat. No. 4,621,100, discloses substituted cyclopentanes useful for bone formation activity. U.S. Pat. No. 5,216,183, discloses cyclopentanones useful for bone formation activity.

Sodium fluoride may be used in combination with the compounds of this invention. The term sodium fluoride refers to sodium fluoride in all its forms (e.g., slow release sodium fluoride, sustained release sodium fluoride). Sustained release sodium fluoride is disclosed in U.S. Pat. No. 4,904,478. The activity of sodium fluoride is readily determined by those skilled in the art of biological protocols.

Bone morphogenetic protein may be used in combination with the compounds of this invention (e.g., see Ono et al., Promotion of the Osteogenetic Activity of Recombinant Human Bone Morphogenetic Protein by Prostaglandin E₁, Bone 19(6): 581-588 (1996)).

Any parathyroid hormone (PTH) may be used in combination with the compound of this invention. The term parathyroid hormone refers to parathyroid hormone, fragments or metabolites thereof and structural analogs thereof which can stimulate bone formation and increase bone mass. Also included are parathyroid hormone related peptides and active fragments and analogs of parathyroid related peptides (see PCT publication No. WO 94/01460). Such bone anabolic functional activity is readily determined by those skilled in the art of standard assays. A variety of these compounds are described and referenced below. However, other parathyroid hormone will be known to those skilled in the art. Exemplary parathyroid hormones are disclosed in the following references. “Human Parathyroid Peptide Treatment of Vertebral Osteoporosis”, Osteoporosis Int., 3, (Supp 1): 199-203. “PTH 1-34 Treatment of Osteoporosis with Added Hormone Replacement Therapy: Biochemical, Kinetic and Histological Responses” Osteoporosis Int. 1: 162-170.

Any growth hormone or growth hormone secretagogue may be used in combination with the compounds of this invention. The term growth hormone secretagogue refers to a compound which stimulates the release of growth hormone or mimics the action of growth hormone (e.g., increases bone formation leading to increased bone mass). Such actions are readily determined by those skilled in the art of standard assays well known to those of skill in the art. A variety of these compounds are disclosed in the following published PCT patent applications: WO 95/14666; WO 95/13069; WO 94/19367; WO 94/13696; and WO 95/34311. However, other growth hormones or growth hormone secretagogues will be known to those skilled in the art. In particular, a preferred growth hormone secretagogue is N-[1(R)-[1,2-Dihydro-1-methanesulfonylspiro[3H-indole-3,4′-piperidin]-1′-yl)carbonyl]-2-(phenylmethyloxy)ethyl]-2-amino-2-methylpropanamide:MK-667. Other preferred growth hormone secretagogues include 2-amino-N-(2-(3a-(R)-benzyl-2-methyl-3-oxo-2,3,3a,4,6,7-hexahydro-pyrazolo-[4,3-c]pyridin-5-yl)-1-(R)-benzyloxymethyl-2-oxo-ethyl)-isobutyramide or its L-tartaric acid salt; 2-amino-N-(1-(R)-benzyloxymethyl-2-(3a-(R)-(4-fluoro-benzyl)-2-methyl-3-oxo-2,3,3a,4,6,7-hexahydro-pyrazolo[4,3-c]pyridin-5-yl)-2-oxo-ethyl)isobutyramide; 2-amino-N-(2-(3a-(R)-benzyl-3-oxo-2,3,3a,4,6,7-hexahydro-pyrazolo[4,3-c]pyridin-5-yl)-1-(R)benzyloxymethyl-2-oxo-ethyl)isobutyramide; and 2-amino-N-(1-(2,4-difluoro-benzyloxymethyl)-2-oxo-2-(3-oxo-3a-pyridin-2-ylmethyl-2-(2,2,2-trifluoro-ethyl)-2,3,3a,4,6,7-hexahydro-pyrazolo[4,3-c]pyridin-5-yl)-ethyl)-2-methyl-propionamide.

The other therapeutic agent can be a steroid or a non-steroidal anti-inflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene-acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

For arthritis, inflammation-mediated bone loss and other disorders that have an inflammatory component, preferred conventional treatments for use in combination therapy with the compounds and compositions of this invention include (without limitation) naproxen sodium (Anaprox® and Anaprox® DS, Roche), flurbiprofen (Ansaid®; Pharmacia), diclofenac sodium+misoprostil (Arthrotec®, Searle), valdecoxib (Bextra®, Pharmacia), diclofenac potassium (Cataflam® and Voltaren®, Novartis), celecoxib (Celebrex®, Pharmacia), sulindac (Clinoril®, Merck), oxaprozin (Daypro®, Pharmacia), salsalate (Disalcid®, 3M), diflunisal (Dolobid®, Merck), naproxen sodium (EC Naprosyn®, Roche), piroxicam (Feldene®, Pfizer), indomethacin (Indocin® and Indocin SR®, Merck), etodolac (Lodine® and Lodine XL®, Wyeth), meloxicam (Mobic®, Boehringer Ingelheim), ibuprofen (Motrin®, Pharmacia), naproxen (Naprelan®, Elan), naproxen (Naprosyn®, Roche), ketoprofen (Orudis® and Oruvail®, Wyeth), nabumetone (Relafen®, SmithKline), tolmetin sodium (Tolectin®, McNeil), choline magnesium trisalicylate (Trilisate®, Purdue Fredrick), and rofecoxib (Vioxx®, Merck).

In any case where pain in a component of the target disorder, the other therapeutic agent can be an analgesic. Useful analgesics include, but are not limited to, phenacetin, butacetin, acetaminophen, nefopam, acetoamidoquinone, and mixtures thereof.

For use against osteoporosis, Paget's disease and other disorders associated with bone deterioration, preferred conventional agents that mayu be used in combination with compounds and compositions of this invention include (without limitation) bisphosphonates (such as etidronate (Didronel®, Procter & Gamble), pamidronate (Aredia®, Novartis), and alendronate (Fosamax®, Merck)), tiludronate (Skelid®), Sanofi-Synthelabo, Inc.), risedronate (Actonel®, Procter & Gamble/Aventis), calcitonin (Miacalcin®), estrogens (Climara®, Estrace®, Estraderm®, Estratab®, Ogen®, Ortho-Est®, Vivelle®, Premarin®, and others) estrogens and progestins (Activella™, FemHrt®, Premphase®, Prempro®, and others), parathyroid hormone and portions thereof, such as teriparatide (Forteo®, Eli Lilly and Co.), selective estrogen receptor modulators (SERMs) (such as raloxifene (Evista®)) and treatments currently under investigation (such as other parathyroid hormones, sodium fluoride, vitamin D metabolites, and other bisphosphonates and selective estrogen receptor modulators).

The foregoing and other useful combination therapies will be understood and appreciated by those of skill in the art. Potential advantages of such combination therapies include the ability to use less of each of the individual active ingredients to minimize toxic side effects, synergistic improvements in efficacy, improved ease of administration or use and/or reduced overall expense of compound preparation or formulation.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description and the examples that follow, one skilled in the art can easily ascertain the essential characteristics of the present invention, and without departing from the spirit and scope thereof, can make various changes and modifications of the invention to adapt it to various usages and conditions. For example, the compounds of this Invention may be used as research tools (for example, to isolate new targets for performing drug discovery). The compounds may, for instance, be radiolabelled for imaging tissue or organs or be used to form bioconjugates for affinity assays. These and other uses and embodiments of the compounds and compositions of this invention will be apparent to those of ordinary skill in the art.

The invention is further defined by reference to the following examples describing in detail the preparation of compounds of, the invention. It will be apparent to those skilled in the art that many modifications, both to materials and methods, may be practiced vvithout departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### Synthesis

The compounds of this invention can be prepared by methods well known in the art, as well as by the synthetic routes disclosed herein. For example, a compound of this invention can be prepared by using 2,4,6-trichloro-pyrimidine as a starting material. The three chloro groups can be displaced by various substitutes. More specifically, first chloro group (e.g., at position 6) can react with, e.g., morpholine, to form a morpholinyl pyrimidine. 2-Aryl and 2-alkylpyrimidinde dichloro compounds can also be prepared by reacting an amidine with a malonic ester followed by treatment with phosphorous oxychloride. Second chloro group can be replaced by reacting with a nucleophile, such as an alcohol in the presence of base, e.g., sodium hydride. In other examples, a compound of formula (I), (I′), or (I″), wherein Y is CH₂ (e.g., Compound 1), can be prepared by reacting the pyrimidine chloride with a Grignard reagent, an organotin reagent, an organocopper reagent, an organoboric acid, or an organozinc reagent in the presence of an organopalladium compound as a catalyst. Isomeric forms may be produced. The desired isomeric product can be separated from others by, e.g., high performance liquid chromatography. Third chloro group undergoes a displacement reaction with, e.g., hydrazine, and the primary amine of the coupled hydrazine moiety further reacts with an aldehyde, e.g., indole-3-carboxaldehyde to form a hydrazone linkage. Thus, a compound of this invention is obtained. If preferred, other types of linkages can be prepared by similar reactions. Sensitive moieties on a pyrimidinyl intermediate and a nucleophile can be protected prior to coupling.

The compounds described above can be prepared by methods well known in the art, as well as by the synthetic routes disclosed herein. For example, a triazine compound of this invention (e.g., Compound 101) can be prepared in a stepwise manner by using cyanuric chloride as a starting material and replacing its three chloro groups with various substitutes by the methods described above. Due to the symmetry of cyanuric chloride, the order of displacement is not of particular importance. For example, a chloro group of cyanuric chloride can be substituted with a nucleophile X—R₁—H, wherein X is O or S, thus forming an ether linkage. In another example, a compound of formula (I′), wherein Y is CH₂ (e.g., Compound 107), can be prepared by reacting the cyanuric chloride with a Grignard reagent, an organotin reagent, an organoboric acid, an organocopper reagent or an organozinc reagent in the presence of an organopalladium compound as a catalyst. If preferred, other types of linkages can be prepared by similar nucleophilic reactions. Sensitive moieties on the triazinyl intermediates and on the nucleophiles can be protected prior to coupling. For suitable protecting groups, see, e.g., Greene (1981) Protective Groups in Organic Synthesis, John Wiley & Sons, Inc., New York. A triazine compound thus synthesized can be further purified by flash column chromatography, high performance liquid chromatography, or crystallization.

The bicyclic compounds of this invention can be prepared by methods well known in the art, as well as by the synthetic routes disclosed herein. For example, a purine compound (i.e., each of U and V is N, A is NR^{e}, and B is N. U, V, A, B and R^{e} are as defined in Summary) is prepared by using 2,4,8-trichloropurine as a starting material. The three chloro groups can be displaced by various substituents. More specifically, the most reactive chloro group (i.e., chloro at position 4) is substituted with a morpolino group to form morpholinopurine. Further reaction of morpholinopurine with a primary or secondary aromatic amine affords a desired compound. In another example, a purine compound is synthesized by reacting 4,8-dichloropuine subsequently with morpholine, a primary or secondary amine, halogen (e.g., bromine), and another primary or secondary amine, or an aryloxy agent (e.g., sodium phenoxide). In further another example, a compound described in Summary is prepared by reacting 3,4-diaminopyrimidine with an arylisocyanate (e.g., m-tolyl isocyanate) or aryidithioiminocarbonate (e.g., dimethyl N-(m-tolyl)-dithioiminocarbonate).

The chemicals used in the above-described synthetic routes may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the pyrimidine compounds. In addition, various synthetic steps may be performed in an alternate sequence or order to give the desired compounds. Synthetic chemistry transformations and protecting group methodologies (protection and deprotection) useful in synthesizing applicable pyrimidine compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A compound thus obtained can be further purified by conventional methods known to those of skill in the art, including without limitation, flash column chromatography, high performance liquid chromatography, and crystallization.

### Synthesis of Exemplary Compounds

### Example 1

### Preparation of Compound 1: N-{2-[3-(3,4-dimethoxy-phenyl)-propyl]-6-morpholin-4-yl-pyrimidin-4-yl}-N′-(1H-indol-3-ylmethylene)-hydrazine

To a solution of 3-(3,4-dimethoxyphenyl)-propyl iodide (1.224 g, 4.0 mmol) in 20 mL dry THF, highly active zinc (suspension in THF, Rieke metal from Aldrich, 5.2 mL 0.05 g/mL, 4.0 mmol) was added to obtain a mixture. The mixture was stirred at room temperature overnight. 2,4-dichloro-6-morpholinopyrimidine (0.932 g, 4.0 mmol) and trans-benzyl-(chloro)-bis-(triphenylphosphine)palladium(II) (0.03 g, 0.04 mmol) were added to the mixture, and stirred at 60° C. for 2 days. After routine workup, 4-chloro-2-[3-(3,4-dimethoxyphenyl)propyl]-6-morpholinopyrimidine (0.34 g, 0.90 mmol, 22.4%) was separated from 2-chloro-4-[3-(3,4-dimethoxyphenyl)propyl]-6-morpholinopyrimidine (0.45 g, 1.19 mmol, 30%) by flash chromatography purification.

¹H NMR (300 MHz, CDCl₃), δ (ppm): 6.70-6.80 (m, 3H); 6.32 (s, 1H); 3.87 (s, 3H); 3.85 (s, 3H); 3.73-3.78 (m, 4H); 3.60-3.64 (m, 4H); 2.76 (d, J=7.8 Hz, 2H); 2.63 (d, J=7.5 Hz, 2H); and 2.01-2.12 (m, 2H).

MS (ESI): m/z 380.2 (M+H).

Further, 4-chloro-2-[3-(3,4-dimethoxyphenyl)propyl]-6-morpholinopyrimidine (0.34 g, 0.90 mmol) was reacted with hydrazine (0.29 g, 9 mmol) to obtain 2-[3-(3,4-dimethoxyphenyl)propyl]-4-hydrazino-6-morpholinopyrimidine as a white solid (0.30 g, 0.80 mmol, 89%).

¹H NMR (300 MHz, CDCl₃), δ (ppm): 6.73-6.80 (m, 3H); 5.88 (s, 1H); 5.74 (s, 1H); 3.87 (s, 3H); 3.85 (s, 3H); 3.76-3.79 (m, 4H); 3.69 (d, J=0.6 Hz, 2H); 3.56-3.60 (m, 4H); 2.64 (d, J=7.5 Hz, 4H); and 2.00-2.15 (m, 2H).

MS (ESI): m/z 374.2 (M−H).

A 5 mL methanol solution containing 2-[3-(3,4-dimethoxyphenyl)-propyl]-4-hydrazino-6-morpholinopyrimidine (0.177 g, 0.50 mmol), indole-3-carboxaldehyde (0.073 g, 0.50 mmol), and AcOH (20 mg, cat.) was stirred at 70° C. for 4 hours. Solvent was removed and the crude residue was purified using flash chromatography to give Compound 1 as a light brown solid (0.21 g, 0.42 mmol, 84%).

¹H NMR (300 MHz, CDCl₃), δ (ppm): 8.57 (br s, 1H); 8.45 (br s, 1H); 8.29-8.32 (m, 1H); 8.00 (s, 1H); 7.39-7.43 (m, 2H); 7.23-7.34 (m, 2H); 6.74-6.80 (m, 3H); 6.30 (s, 1H); 3.86 (s, 3H); 3.85 (s, 3H); 3.78-3.84 (m, 4H); 3.67-3.70 (m, 4H); 2.63-2.71 (m, 4H), and 2.03-2.13 (m, 2H).

MS (ESI): m/z 501.2 (M+H).

### Example 2

### Preparation of Compound 2: N-(2-n-butoxy-6-morpholin-4-yl-pyrimidin-4-yl)-N′-(1H-indol-3-ylmethylene)-hydrazine

To a solution of 2,4,6-trichloro pyrimidine (25 g, 136 mmol) in CH₂Cl₂ (500 mL) at −78° C., morpholine (11.89 mL, 136 mmol) was slowly added, followed by DIPEA (25 mL, 143 mmol). The obtained reaction mixture was stirred at −78° C. for 5 h, and then warmed up to room temperature. The reaction mixture was washed with water. The obtained organic phase was dried over Na₂SO₄. The solvent was removed under reduced pressure. The crued residue, 2,4-Dichloro-6-(morpholin-4-yl)pyrimidine, was recrystallized from EtOAc to give white crystals (24.7 g, 77%) 15 g.

¹H NMR (300 MHz, CDCl₃), δ (ppm): 6.40 (s, 1H); and 4.0-3.5 (m, 8H). MS (ESI): m/z 234.0 (M+H).

To a solution of n-butanol (0.633 g, 8.54 mmol) in anhydrous DMF (50 mL) at 0° C. under the N₂, NaH (0.307 g, 12.8 mmol) was added quickly. The obtained suspension was stirred for 0.5 h at 0° C. 2,4-Dichloro-6-(morpholin-4-yl)pyrimidine (2 g, 8.54 mmol) was added to the suspension. After the suspension was warmed to room temperature and stirred for 12 h, the reaction mixture was quenched with ice/brine and extracted with 200 mL EtOAc. The extract was washed with brine, and dried over Na₂SO₄. The solvent was removed under reduced pressure. The crude residue was purified using flash chromatography (silica; EtOAc/Hexane: ⅙) to yield 1.4 g of 2-n-butoxy-4-chloro-6-(morpholin-4-yl)pyrimidine (white solid, 60%).

¹H NMR (300 MHz, CDCl₃), δ (ppm): 6.20 (s, 1H); 4.26 (t, J=6.6 Hz, 2H); 3.78-3.70 (m, 4H); 3.66-3.56 (m, 4H); 1.80-1.68 (m, 2H); 1.54-1.40 (m, 2H); and 0.96 (t, J=6.9, 3H).

MS (ESI): m/z 272.1 (M+H).

To a solution of 2-n-butoxy-4-chloro-6-(morpholin-4-yl)pyrimidine (1.38 g, 5.1 mmol) in dioxane (50 ml), anhydrous hydrazine (1.6 mL, 50 mmol) was added. The obtained reaction mixture was heated to 95° C., and stirred for 12 h under N2. After cooling to room temperature, the reaction mixture was quenched with ice-brine and extracted with EtOAc (200 mL). The organic extract was washed with brine, water, and dried over Na₂SO₄. The solvent was removed under reduced pressure. The crude residue was recrystallized from methanol to obtain 2-n-butoxy-4-hydrazino-6-(morpholin-4-yl)pyrimidine as white crystals (1.10 g, 81%).

¹H NMR (300 MHz, CDCl₃), δ (ppm): 5.89 (br s, 1H), 5.49 (s, 1H), 4.26 (t, J=6.6, 2H), 3.84-3.78 (m, 6H), 3.62-3.47 (m, 4H), 1.82-1.67 (m, 2H), 1.55-1.42 (m, 2H), and 0.96 (t, J=6.9, 3H);

MS (ESI): m/z 268.2 (M+H).

To a solution of 2-n-butoxy-4-hydrazino-6-(morpholin-4-yl)pyrimidine (200 mg, 0.748 mmol) in MeOH (20 mL), indole-3-carboxaldehyde (108.6 mg, 0.748 mmol) and acetic acid (a drop) were added sequentially. The obtained reaction mixture was stirred at room temperature for 12 h. White precipitate was formed, collected, and washed with 2 mL methanol to give 200 g of Compound 2 (68%).

¹H NMR (300 MHz, CDCl₃), δ (ppm): 8.36 (br s, 1H), 8.30 (dd, J=6.6, 1.8, 1H), 8.05 (s, 1H), 8.00 (s, 1H), 7.44-7.40 (m, 2H), 7.33-7.24 (m, 2H), 6.13 (s, 1H), 4.26 (t, 2H, J=6.6), 3.84-3.78 (m, 4H), 3.70-3.64 (m, 4H), 1.80-1.70 (m, 2H), 1.54-1.42 (m, 2H), and 0.96 (t, J=6.9, 3H);

MS (ESI): m/z 395.2 (M+H).

### Example 3

### Preparation of Compound 3: N-(2-(4-hydroxybutyl)-6-morpholin-4-yl-pyrimidin-4-yl)-N′-(1H-indol-3-ylmethylene)-hydrazine

A mixture of 4-ethoxy-4-oxo-butylzinc bromide (50 mL 0.5M in THF, 25 mmol), 2,4-dichloro-6-morpholinopyrimidine (4.68 g, 20.0 mmol) and trans-benzyl(chloro)bis(triphenylphosphine)palladium(II) (0.15 g, 0.2 mmol) in THF (total volume 80 mL) was stirred at 60° C. for 2 days. After routine workup, flash chromatography purification was performed to obtain 4-chloro-2-(4-ethoxy-4-oxo-butyl)-6-morpholinopyrimidine as a white solid (2.073 g, 6.60 mmol, 33.0%).

To a solution of 4-chloro-2-(4-ethoxy-4-oxo-butyl)-6-morpholinopyrimidine (1.108 g, 3.54 mmol) in 50 mL THF at −78° C., a diisobutylaluminum hydride (DIBAL) solution (4.72 mL 1.5 M in Toluene, 7.08 mmol) was slowly added. After addition, the obtained reaction mixture was warmed up slowly to 0° C. and kept at 0° C. for 10 min. After routine workup, flash chromatography was performed to obtain 4-chloro-2-(4-hydroxybutyl)-6-morpholinopyrimidine (0.76 g, 2.80 mmol, 79%) as light yellow solid.

¹H NMR (300 MHz, CDCl₃), δ (ppm): 6.33 (s, 1H), 3.76-3.79 (m, 4H); 3.61-3.68 (m, 6H); 2.76 (t, J=7.8 Hz, 2H); 1.81-1.91 (m, 2H); and 1.60-1.74 (m, 3H).

MS (ESI): m/z 370.2 (M+H).

Following the typical procedure, 4-chloro-2-(4-hydroxybutyl)-6-morpholinopyrimidine (0.542 g, 2.00 mmol, 1.00 equiv.) was reacted with hydrazine and indole-3-carboxaldehyde to give Compound 3 as an off-white solid (0.75 g, 1.90 mmol, 95%).

¹H NMR (300 MHz, DMSO-d₆), δ (ppm): 11.47 (s, 1H); 10.64 (s, 1H); 8.25 (s, 1H); 8.18 (d, J=6.6 Hz, 1H); 7.71 (s, 1H); 7.43 (d, J=8.4 Hz, 1H); 7.17-7.20 (m, 2H); 6.16 (s, 1H), 4.37 (t, J=4.8 Hz, 1H); 3.72 (br s, 4H); 3.55 (br s, 4H); 3.41-3.45 (m, 2H); 2.49-2.54 (m, 2H), 1.66-1.76 (m 2H); and 1.42-1.53 (m 2H).

MS (ESI): m/z 395.1 (M+H).

### Example 4

### Preparation of Compound 4: N-[2-(2-[1,3]dioxan-2-yl-ethyl)-6-morpholin-4-yl-pyrimidin-4-yl]-N′-(1H-indol-3-ylmethylene)-hydrazine

Compound 4 was prepared in a similar manner as described in Example 1. ¹H NMR (300 MHz, DMSO-d₆), δ (ppm): 11.46 (s, 1H); 10.64 (s, 1H); 8.25 (s, 1H); 8.18 (d, J=6.6 Hz, 1H); 7.71 (s, 1H); 7.43 (d, J=6.0 Hz, 7.5 Hz, 1H); 7.16-7.19 (m, 2H); 6.15 (s, 1H), 4.58 (t, J=5.1 Hz, 1H); 4.00 (dd, J=11.4 Hz, 4.5 Hz, 2H); 3.64-3.72 (m, 6H); 3.54 (br s, 4H); 2.50-2.59 (m, 2H); 1.80-1.94 (m, 3H), and 1.33 (d, J=9.6 Hz, 1H).

MS (ESI): m/z 437.2 (M+H).

### Example 5

### Preparation of Compound 5: N-(1H-indol-3-ylmethylene)-N′-[2-(3-methoxy-propyl)-6-morpholin-4-yl-pyrimidin-4-yl]-hydrazine

Following the procedure for the synthesis of N-(2-(4-Hydroxybutyl)-6-morpholin-4-yl-pyrimidin-4-yl)-N′-((H-indol-3-yl methylene)-hydrazine (Corm pound 3),4-chloro-2-(3-hydroxypropyl)-6-morpholinopyrimidine (0.81 g, 3.15 mmol) was synthesized, methylated with sodium hydride (0.48 g, 6.30 mmol) for 10 min, and MeI (0.895 g, 6.30 mmol) for 5 h in 30 mL THF at 0° C. to give 4-chloro-2-(3-methoxypropyl)-6-morpholinopyrimidine as colorless viscous oil (0.792 g, 3.03 mmol, 96%).

¹H NMR (300 MHz, CDCl₃), δ (ppm): 6.32 (s, 1H), 3.75-3.79 (m, 4H); 3.61-3.64 (m, 4H); 3.44 (t, J=6.6 Hz, 2H); 3.34 (s, 3H); 2.78 (t, J=7.8 Hz, 2H); and 2.00-2.09 (m, 2H).

MS (ESI): m/z 262.1 (M+H).

Following the typical procedure, 4-chloro-2-(3-methoxypropyl)-6-morpholinopyrimidine (0.783 g, 3.00 mmol) was treated with hydrazine and indole-3-carboxaldehyde sequentially to yield 0.89 g of Compound 5 (2.26 mmol, 75%).

¹H NMR (300 MHz, DMSO-d₆), δ (ppm): 11.46 (s, 1H); 10.64 (s, 1H); 8.26 (s, 1H); 8.17-8.20 (m, 1H); 7.72 (d, J=2.4 Hz, 1H); 7.43 (dd, J=6.0 Hz, 2.4 Hz, 1H); 7.15-7.21 (m, 2H); 6.16 (s, 1H), 3.70-3.73 (m, 4H); 3.52-3.56 (m, 4H); 3.37 (t, J=6.9 Hz, 2H); 3.23 (s, 3H); 2.50-2.57 (m, 2H), and 1.88-1.97 (m, 2H).

MS (ESI): m/z 395.2 (M+H).

### Example 6

### Preparation of Compound 6: 3-{4-[N′-(1H-indol-3-ylmethylene)-hydrazino]-6-morpholin-4-yl-pyrimidin-2-ylsulfanyl}-propan-1-ol

Compound 6 was prepared in a similar manner as described in Example 2.

¹H NMR (300 MHz, DMSO-d₆), δ (ppm): 11.48 (s, 1H); 10.68 (s, 1H); 8.26 (s, 1H); 8.15-8.18 (m, 1H); 7.73 (d, J=2.1 Hz, 1H); 7.42-7.44 (m, 1H); 7.16-7.20 (m, 2H); 6.04 (s, 1H), 4.53 (t, J=5.1 Hz, 1H); 3.65-3.71 (m, 4H); 3.48-3.56 (m, 6H); 3.06 (t, J=7.2 Hz, 2H), and 1.76-1.85 (m, 2H).

MS (ESI): m/z 413.1 (M+H).

### Example 7

### Preparation of Compound 7: 3-{2-[N′-(1H-indol-3-ylmethylene)-hydrazino]-6-morpholin-4-yl-pyrimidin-4-ylsulfanyl}-propan-1-ol

Compound 7 was prepared in a similar manner as described in Example 2.

¹H NMR (300 MHz, DMSO-d₆), δ (ppm): 11.34 (s, 1H); 10.48 (s, 1H); 8.45 (d, J=7.8 Hz, 1H); 8.25 (s, 1H); 7.64 (d, J=2.7 Hz, 1H); 7.40 (d, J=8.1 Hz, 1H); 7.05-7.19 (m, 2H); 6.08 (s, 1H), 4.60 (t, J=5.1 Hz, 1H); 3.50-3.68 (m, 10H); 3.20-3.30 (m, 2H); and 1.78-1.86 (m, 2H).

MS (ESI): m/z 413.1 (M+H).

### Example 8

### Preparation of Compound 8: N-[2-(2,2-dimethyl-[1,3]dioxolan-4-yl methoxy)-6-morpholin-4-yl-pyrimidin-4-yl]-N′-(1H-indol-3-ylmethylene)-hydrazine

Compound 8 was prepared in a similar manner as described in Example 2.

¹H NMR (300 MHz, CDCl₃), δ (ppm): 8.38 (br s, 1H); 8.30 (dd, J=7.2, 1.8, 1H), 8.02 (br s, 1H); 8.00 (s, 1H); 7.44-7.41 (m; 2H); 7.32-7.26 (m, 2H); 6.14 (s, 1H); 4.51-4.42 (m, 2H); 4.22-4.12 (m, 2H); 3.96-3.91 (m, 1H); 3.84-3.79 (m, 4H); 3.70-3.64 (m, 4H); 1.47 (s, 3H); and 1.38 (s, 3H).

MS (ESI): m/z 453.2 (M+H).

### Example 9

### Preparation of Compound 9: N-{2-[2-(3,4-dimethoxy-phenyl)-ethoxy]-6-morpholin-4-yl-pyrimidin-4-yl}-N′-(1H-indol-3-ylmethylene)-hydrazine

Compound 9 was prepared in a similar manner as described in Example 2.

¹H NMR (300 MHz, CDCl₃), δ (ppm): 8.43 (bs, 1H); 8.30 (d, J=7.5 Hz 1H); 8.2 (bs, 1H); 8.02 (d, J=2.7 Hz, 1H); 7.46-7.40 (m, 2H); 7.30-7.26 (m, 2H); 6.82 (d, J=1 Hz, 3H); 4.45 (d, J=3.6 Hz, 1H); 4.45 (t, J=5.2 Hz, 2H); 3.87 (d, J=3.9 Hz, 3H); 3.86 (d, J=3.9 Hz, 3H); 3.81 (s, 4H); 3.67 (s, 4H); and 3.04 (t, J=5.0 Hz, 2H).

MS (ESI): m/z 503.2 (M+H).

### Example 10

### Preparation of Compound 10: N-(1H-indol-3-ylmethylene), N′-[6-morpholin-4-yl-2-(2-pyridin-2-yl-ethoxy)-pyrimidin-4-yl]-hydrazine

Compound 10 was prepared in a similar manner as described in Example 2.

¹H NMR (300 MHz, CDCl₃), δ (ppm): 9.3 (bs, 1H); 8.66 (s, 1H); 8.55-8.53 (m, 1H); 8.28-8.26 (m, 1H); 8.04 (s, 1H); 7.62-7.57 (m, 1H); 7.41-7.10 (r, 6H); 6.08 (s, 1H); 4.64 (t, J=6.6 Hz, 2H); 3.76 (s, 4H); 3.62 (s, 4H); and 3.26 (t, J=6.6 Hz, 2H).

MS (ESI): m/z 444.2 (M+H).

### Example 11

### Preparation of Compound 11: N-(1H-indol-3-ylmethylene)-N′-[6-morpholin-4-yl-2-(3-Pyridin-2-yl-propyl)-pyrimidin-4-yl]-hydrazine

Compound 11 was prepared in a similar manner as described in Example 1.

¹H NMR (300 MHz, DMSO-d₆), δ (ppm): 11.47 (s, 1H); 10.65 (s, 1H); 8.50 (d, J=4.5 Hz, 1H); 8.26 (s, 1H); 8.20-8.18 (m, 1H); 7.72-7.68 (m, 2H); 7.45-7.42 (m, 1H); 7.29-7.18 (m, 4H); 6.17 (s, 1H); 3.73 (s, 4H); 3.5 (s, 4H); 2.79 (t, J=7.5 Hz, 2H); 2.58-2.51 (m, 2H); and 2.18-2.06 (m, 2H).

MS (ESI): m/z 442.2 (M+H).

### Example 12

### Preparation of Compound 12: N-(3-methyl-benzylidene)-N′-[6-morpholin-4-yl-2-(2-pyridin-2-yl-ethoxy)-pyrimidin-4-yl]-hydrazine

Compound 12 was prepared in a similar manner as described in Example 2.

¹H NMR (300 MHz, CDCl₃), Λ (ppm): 8.55-8.48 (m, 2H); 7.71 (s, 1H); 7.65-7.55 (m, 1H); 7.49-7.42 (m, 2H); 7.30-7.15 (m, 4H); 6.08 (s, 1H); 4.64 (t, J=6.6 Hz, 2H); 3.81-3.75 (m, 4H); 3.64-3.61 (m, 4H); 3.25 (ti J=7.0 Hz, 2H); and 2.38 (s, 3H).

MS (ESI): m/z 419.2 (M+H).

### Example 13

### Preparation of Compound 13: N-(3-ethyl-benzylidene)-N′-[6-morpholin-4-yl)-2-(2-pyridin-2-yl-ethoxy)-pyrimidin-4-yl]-hydrazine

Compound 13 was prepared in a similar manner as described in Example 2.

¹H NMR (300 MHz, CDCl₃), δ (ppm): 8.58-8.50 (m, 1H); 8.43 (s, 1H); 7.95 (s, 1H); 7.64-7.58 (m, 2H); 7.30-7.25 (m, 1H); 7.18-7.05 (m, 3H); 6.07 (s, 1H); 4.65 (t, J=6.9 Hz, 2H); 3.80-3.76 (m, 4H); 3.64-3.61 (m, 4H); 3.26 (t, J=6.9 Hz, 2H); 2.40 (q, J=7.6 Hz, 2H); and 1.45 (t, J=7.6 Hz, 3H).

MS (ESI): m/z 433.3 (M+H).

### Example 14

### Preparation of Compound 14: N-(3-methyl-benzylidene)-N′-[6-morpholin-4-yl-2-(3-pyridin-2-yl-propyl)-pyrimidin-4-yl]-hydrazine

Compound 14 was prepared in a similar manner as described in Example 1.

¹H NMR (300 MHz, CDCl₃), δ (ppm): 9.6 (bs, 1H); 8.53 (d, J=4.5 Hz, 1H); 7.76 (s, 1H); 7.56 (t, J=6 Hz, 1H); 7.49-1.47 (m, 2H); 7.28 (m, 1H); 7.18-7.06 (m, 3H); 6.26 (s, 1H); 3.81-3.79 (m, 4H); 3.69-3.67 (m, 4H); 2.89 (t, J=7.8 Hz, 2H); 2.71 (t, J=7.5 Hz, 2H); 2.39 (s, 3H); and 2.22 (t, J=7.5 Hz, 2H).

MS (ESI): m/z 417.2 (M+H).

### Example 15

### Preparation of Compound 15: N-[6-morpholin-4-yl-2-(2-pyridin-2-yl-ethoxy)-pyrimidin-4-yl]-N′-(1-m-tolyl-ethylidene)-hydrazine

Compound 15 was prepared in a similar manner as described in Example 2.

¹H NMR (300 MHz, CDCl₃), δ (ppm): 8.56 (bs, 1H), 7.66-7.46 (m, 4H), 7.32-7.26 (m, 2H), 7.16-7.14 (m, 2H), 6.44 (s, 1H), 4.69 (t, J=6.9 Hz, 2H), 3.80-3.77 (m, 4H), 3.63-3.60 (m, 4H), 3.31 (t, J=6.9 Hz, 2H), 2.39 (s, 3H).

MS (ESI): m/z 433.2 (M+H).

### Example 16

### Preparation of Compound 16: N-[1-(1H-indol-3-yl)-ethylidene]-N′-[6-morpholin-4-yl-2-(2-pyridin-2-yl-ethoxy)-pyrimidin-4-yl]-hydrazine

Compound 16 was prepared in a similar manner as described in Example 2.

¹H NMR (300 MHz, CDCl₃), δ (ppm): 9.35 (bs, 1H); 8.54 (dd, J=0.9, 4.2 Hz, 1H); 8.33 (d, J=7.5 Hz, 1H); 7.93 (s, 1H); 7.58 (t, J=7.2 Hz, 1H); 7.36-7.33 (m, 2H); 7.27-7.120 (m, 4H); 6.49 (s, 1H); 4.68 (t, J=7.2 Hz, 2H); 3.76-3.73 (m, 4H); 3.60-3.57 (m, 4H); 3.50 (s, 3H); and 3.33-3.28 (t, J=7.0 Hz, 2H).

MS (ESI): m/z 458.2 (M+H).

### Example 17

### Preparation of Compound 17: 3-Methyl-benzaldehyde O-[6-morpholin-4-yl-2-(2-pyridin-2-yl-ethoxy)-pyrimidin-4-yl]-oxime

Compound 17 was prepared in a similar manner as described in Example 2.

¹H NMR (300 MHz, CDCl₃), δ (ppm): 8.56-8.53 (m, 1H); 8.45 (s, 1H); 7.62-7.50 (m, 3H); 7.38-7.26 (m, 3H); 7.18-7.10 (m, 1H); 6.17 (s, 1H); 4.68 (t, J=6.9 Hz, 2H); 3.80-3.76 (m, 4H); 3.67-3.64 (m, 4H); 3.29 (t, J=6.9 Hz, 2H); and 2.41 (s, 3H).

MS (ESI): m/z 420.1 (M+H).

### Example 18

### Preparation of Compound 18: 1H-indole-3-carbaldehyde O-[6-morpholin-4′-yl-2-(2-pyridin-2-yl-ethoxy)-pyrimidin-4-yl]-oxime

Compound 18 was prepared in a similar manner as described in Example 2.

¹H NMR (300 MHz, DMSO-d₆), δ (ppm): 11.82 (bs, 1H); 8.81 (s, 1H); 8.50 (d, J=4.5 Hz, 1H); 8.04 (d, J=6.9 Hz, 1H); 7.93 (s, 1H); 7.72 (t, J=6.9 Hz, 1H); 7.49 (d, J=6.9 Hz, 1H); 7.33 (d, J=7.8 Hz, 1H); 7.30-7.18 (m, 3H); 6.22 (s, 1H); 4.57 (t, J=6.3 Hz, 2H); 3.67 (s, 4H); 3.56 (s, 4H); and 3.15 (t, J=6.3 Hz, 2H).

MS (ESI): m/z 445.2 (M+H).

### Example 19

### Preparation of Compound 19: N-(1H-indol-3-ylmethylene)-N′-{6-morpholin-4-yl-2-[2-(pyridin-3-yloxy)-ethoxy]-pyrimidin-4-yl}-hydrazine

Compound 19 was prepared in a similar manner as described in Example 2.

¹H NMR: (300 MHz, CDCl₃), δ (ppm): 9.20 (br s, 1H); 8.30 (br s, 1H); 8.29 (t, J=3.3 Hz, 1H); 8.18-8.12 (m, 2H); 7.44-7.41 (m, 2H); 7.26-7.18 (m, 5H); 6.08 (s, 1H); 4.66 (t, J=4.8 Hz, 2H); 4.29 (t, J=5.0 Hz, 5H); 3.80-3.76 (m, 4H); and 3.67-3.62 (m, 4H),

MS (ESI): m/z 460.2 (M+H).

### Example 20

### Preparation of Compound 20: N-(3-methyl-benzylidene)-N′-{6-morpholin-4-yl-2-[2-(pyridin-3-yloxy)-ethoxy]-pyrimidin-4-yl}-hydrazine

Compound 20 was prepared in a similar manner as described in Example 2.

¹H NMR (300 MHz, CDCl₃), δ (ppm): 8.55 (s, 1H); 8.34 (br s, 1H); 8.30-8.23 (m, 1H); 7.78 (s, 1H); 7.50-7.47 (m, 2H); 7.32-7.24 (m, 1H); 7.20-7.17 (m, 3H); 6.14 (s, 1H); 4.66 (t, J=5.0 Hz, 2H); 4.35 (t, J=4.8 Hz, 2H); 3.83-3.80 (m, 4H); 3.68-3.65 (m, 4H); and 2.40 (s, 3H).

MS (ESI): m/z 435.2 (M+H).

### Example 21

### Preparation of Compound 21: Butyl-{4-[N′-(1H-indol-3-ylmethylene)-hydrazino]-6-morpholin-4-yl-pyrimidin-2-yl}amine

Compound 21 was prepared in a similar manner as described in Example 2.

¹H NMR (300 MHz, CDCl₃), δ ppm: 8.41 (bs, 1H), 8.33-8.30 (m, 1H), 8.19 (bs, 1H), 7.95 (s, 1H), 7.41-7.37 (m, 2H), 7.29-7.25 (m, 2H), 5.96 (s, 1H), 4.65 (t, J=4 Hz, 1H), 3.83-3.80 (m, 4H), 3.65-3.62 (m, 4H), 3.36 (dd, J=6.3, 13.5 Hz, 2H), 1.60-1.55 (m, 2H), 1.35-1.33 (m, 4H), 0.92-0.87 (m, 3H).

MS (ESI): m/z 408.2 (M+H).

### Example 22

### Preparation of Compound 22: N-(3-Methyl-benzylidene)-N′-[6-morpholin-4-yl-2-(pyridin-3-yloxy)-pyrimidin-4-yl]-hydrazine

To a solution of 3-hydroxypyridine (950 mg, 10 mmol) in anhydrous THF (50 mL) at 0° C. under the nitrogen protection was added NaH (60% in oil) (480 mg, 12 mmol). The suspension was stirred for 0.5 h at 0° C., and 2,4,6-trichloropyrimidine (1.84 g, 10 mmol) was added. After the mixture warmed to room temperature and stirred for 2 h, the reaction was quenched by ice brine and extracted with EtOAc (300 mL). The organic phase was washed with brine, dried (Na₂SO₄), filtered, evaporated in vacuo. The cure product was purified by flash chromatography on a column of silica gel (EtOAc-Hexane, 1:7). The product (1.80 g, 7.4 mmol) in CH₂Cl₂ (150 mL) at 0° C. was added slowly morpholine (2.5 g, 28 mmol). The reaction mixture was stirred at 0° C. for 1 h and another 1 h at room temperature. The mixture was washed with water. The organic phase was dried (Na₂SO₄), filtered and evaporated in vacuo and presented three isomers. The isomers was separated by flash chromatography on a column of silica gel (EtOAc-Hexane, 1:7 and 1:3) to obtain 4-[6-chloro-2-(pyridin-3-yloxy)-pyrimidin-4-yl]-morpholine (320 mg, 14.7%).

¹H NMR (300 MHz, CDCl₃), δ (ppm): 8.51 (d, 1H, J=2.7 Hz), 8.44 (dd, 1H, J=1.5, J=3.3 Hz), 7.53-7.49 (m, 1H), 7.34-7.3 (m, 1H), 6.25 (s, 1H), 3.71-3.67 (m, 4H), 3.51-3.48 (m, 4H).

MS (ESI): m/z 293.1.

To a solution of 4-[6-chloro-2-(pyridin-3-yloxy)-pyrimidin-4-yl]-morpholine (295 mg, 1 mmol) in THF (10 mL) was added anhydrous hydrazine (0.320 ml, 10 mmol) under the nitrogen protection. The mixture was heated at 70° C. for 15 min. After cooling to room temperature, the reaction mixture was quenched by ice brine and extracted with EtOAc (100 mL). The organic phase was washed with brine (10 mL) and water (10 ml×2), dried (Na₂SO₄), filtered, evaporated, and purified by flash chromatography on a column of silica gel (CH₂Cl₂ and CH₂Cl₂-MeOH, 95:5) and to give [6-morpholin-4-yl-2-(pyridin-3-yloxy)-pyrimidin-4-yl]-hydrazine (180 mg) in 62% yield. M/Z (M+1) 289.2

To a solution of [6-Morpholin-4-yl-2-(pyridin-3-yl oxy)-pyrimidin-4-yl]-hydrazine (180 mg) (145 mg, 0.5 mmol) and m-tolylaldehyde (72 mg, 0.6 mmol) in MeOH (10 mL) was added acetic acid (1 drop). The reaction mixture was stirred at room temperature for 12 h and white solid was precipitated. The resulting precipitate was collected by filtration and washed with little amount of metanol and to give 125 mg of Compound 22 in 64% yield.

¹H NMR (300 MHz, CDCl₃), δ (ppm): 8.71 (s, 1H), 8.57 (d, 1H, J=2.4 Hz), 8.44 (dd, 1H, J=1.5, 3.2 Hz), 7.78 (s, 1H), 7.56-7.52 (m, 1H), 7.46-7.43 (m, 2H), 7.34-7.26 (m, 2H), 7.17 (d, 1H, J=8.1 Hz), 6.17 (s, 1H), 3.76-3.73 (m, 4H), 3.57-3.54 (m, 4H), 2.38 (s, 3H).

MS (ESI): m/z 391.2.

### Example 23

### Preparation of Compound 23: N-(3-Methylbenzlidene)-N′-(5-methyl-6-morpholin-4-yl-2-phenylpyrimidin-4-yl)hydrazine

Benzamidine hydrochloride (7.06 g, 0.045 mol) and dimethyl methylmalonate (6.0 g, 0.041 mol) were dissolved in methanol (100 mL). Sodium methoxide (21.5 mL, 0.099 mmol, 25 wt % solution in methanol) was added and the solution was stirred at room temperature for 18 h. The volume of solvent was reduced to approximately 50 mL under reduced pressure, then poured onto ice water. This solution was neutralized with HOAc which produced a white precipitate. This precipitate was collected and dried to produce a white solid (6.1 g, 74%).

¹H NMR (DMSO-d₆) δ (ppm) 1.68 (s, 3H), 7.70-7.87 (m, 3H), 8.21 (d, J=8.4 Hz).

MS (ESI): m/z 203.1 (M+H)⁺

5-Methyl-2-phenyl-pyrimidine-4,6-diol (3.3 g, 0.016 mol) and POCl₃ were heated to 60 C for 3 hrs. The solution was allowed to cool to room temperature then poured onto ice. The resultant white precipitate was filtered and dried to produce the desired compound as a white solid (810 mg, 21%).

¹H NMR (DMSO-d₆) δ (ppm) 2.40 (5, 3H), 7.51-7.56 (m, 3H), 8.23 (d, 8.4 Hz).

MS (ESI): m/z 239.1 (M+H)⁺

4,6-Dichloro-5-methyl-2-phenylpyrimidine (2.5 g, 0.010 mol) and morpholine (2.93 g, 0.031 mol) were dissolved in THF (50 mL) and heated to reflux for 3 hrs. The solution was allowed to cool then EtOAc (100 mL) and water (100 mL) were added. The EtOAc layer was washed with water (3×100 mL), dried over MgSO₄, filtered and solvent was removed under reduced pressure. The resultant solid was used without further purification (2.66 g, 92%).

MS (ESI): m/z 298.1 (M+H)⁺

4-(6-Chloro-5-methyl-2-phenylpyrimidin-4-yl)morpholine (439 mg, 1.51 mmol) was dissolved in THF (50 mL). Hydrazine (0.25 mL, 7.96 mmol) was added and the solution was heated to reflux for 18 hrs. The reaction was allowed to cool the solvent was removed under reduced pressure. EtOAc (100 mL) and water (100 mL) were added. The EtOAc layer was washed with water (3×100 mL), dried over MgSO₄, filtered and solvent was removed under reduced pressure to produce a white solid (374 mg). This solid was redissolved in THF (50 mL) and m-tolualdehyde (157 mg, 1.31 mmol) was added. The solution was heated to reflux for 4 hrs then allowed to cool. Solvent was removed under reduced pressure then EtOAc (100 mL) and water (100 mL) were added. The EtOAc layer was washed with water (3×100 mL), dried over MgSO₄, filtered and solvent was removed under reduced pressure. The crude product was purified by silcagel column chromatography, eluting with 25% EtOAc/hexane to produce the pure desired product as a yellow solid (313 mg, 53%).

¹H NMR (DMSO-d₆) δ (ppm) 2.26 (s, 3H), 2.36 (s, 3H), 3.35 (m, 4H), 3.75-3.78 (m, 4H), 7.20 (d, J=6.9 Hz), 7.33 (t, J=6.9 Hz), 7.47-7.52 (m, 5H), 8.19 (s, 1H), 8.35-8.38 (m, 2H), 10.60 (s, 1H).

MS (ESI): m/z 388.3 (M+H)⁺

### Example 24

### Preparation of Compound 24: N-(3-methyl-benzylidene)-N′-(2-Phenyl-6-thiomorpholin-4-yl-pyrimidin-4-yl)-hydrazine

Compound 24 was prepared in a similar manner as described in Example 23.

¹H-NMR (DMSO-d₆) δ 2.36 (s, 3H), 2.76 (s, 4H), 4.07 (s, 4H), 6.36 (s, 1H), 7.19 (d, J=8.1 Hz), 7.32 (t, J=8:1 Hz), 7.47-7.57 (m, 5H), 8.09 (s, 1H), 8.30-8.31 (m, 1H), 11.02 (s, 1H).

MS (ESI): m/z 389.1.

### Example 25

### Preparation of Compound 25: (2,3-Dimethyl-1H-indole-5-yl)-{6-morpholin-4-yl-2-[2-(pyridin-3-yloxy)-ethoxy]-pyrimidin-4-yl}-amine

To a solution of 2-(pyridin-3-yloxy)-ethanol (3.48 g, 25 mmol) in 40 mL of anhydrous THF at room temperature under the N₂, 2,4,6-trichloro pyrimidine (4.56 g, 25 mmol) was added followed by portionwise addition of NaH (60% suspension in oil, 1.1 g, 27.5 mmol). After 30 min of stirring reaction was quenched with water, water layer extracted with EtOAc, combined organic solutions washed with brine and dried over MgSO₄. Purification using flash chromatography (silica; dichloromethane/acetone/methanol: 3/1/0.1) afforded mixture of 4,6-dichloro-2- and 2,6-dichloro-4-[2-(pyridin-3-yloxy)-ethoxy]-pyrimidines (3.72 g, 52%), (NMR ratio 1:1.2) as an oil.

To a solution of the above mixture (3.72 g, 13 mmol) in 20 mL of 1,4-dioxane was added DIPEA (2.49 mL, 14.3 mmol), followed by 2,3-dimethyl-5-amino-indole (2.08 g, 13 mmol) and a mixture was refluxed for 1 hour. Solvent was removed under reduced pressure and reaction mixture was separated using column chromatography (silica; dichloromethane/acetone/methanol: 3/1/0.1) to afford {6-chloro-2-[2-(pyridin-3-yloxy)-ethoxy]-pyrimidin-4-yl}-amine (2.07 g, 39%). An mixture of {4-chloro-6-[2-(pyridin-3-yl oxy)-ethoxy]-pyrimidin-4-yl}-amine and {2-chloro-6-[2-(pyridin-3-yloxy)-ethoxy]-pyrimidin-4-yl}-amine (2.5 g, 47%) was also obtained and used in another reaction.

A solution of (6-chloro-2-[2-(pyridin-3-yloxy)-ethoxy]-pyrimidin-4-yl)-amine (2.07 g, 5.05 mmol) and morpholine (1.32 mL, 15.15 mmol) in 1,4-dioxane was heated at 110° C. for 24 hours. Solvent was removed under reduced pressure and reaction mixture was purified using flash chromatography (silica; dichloromethane/acetone/methanol: 3/1/0.1) to afford Compound 25 (2 g, 86%) as a colorless solid.

¹H NMR (300 MHz, CDCl₃), δ (ppm): 8.34 (br s, 1H), 8.23 (dd, 1H, J=3.6, 2.1), 7.96 (brs, 1H), 7.34-7.21 (m, 4H), 6.98 (dd, 1H, J=8.4, 1.8 Hz), 6.60 (brs, 1H), 5.36 (s, 1H), 4.65 (t, 2H, J=5.1 Hz), 4.34 (t, 2H, J=5.1 Hz), 3.66 (m, 4H), 3.42 (m, 4H), 2.37 (s, 3H), and 2.20 (s, 3H).

MS (ESI): m/z 461.5 (M+H).

### Example 26

### Preparation of Compound 26: (2,3-Dimethyl-1H-indole-5-yl)-{4-morpholin-4-yl-6-[2-(pyridin-3-yloxy)-ethoxy]-pyrimidin-2-yl)-amine

Reaction of a mixture of (4-chloro-6-[2-(pyridin-3-yloxy)-ethoxy]-pyrimidin-4-yl}-amine and 2-chloro-6-[2-(pyridin-3-yloxy)-ethoxy]-pyrimidin-4-yl)-amine (2.5 g, 47%) and (2.5 g, 6.1 mmol) with morpholine was carried out as described in Example 24. Purification by flash chromatography and recrystallization from ether-pentane gave 0.3 g of Compound 26.

¹H NMR (300 MHz, CDCl₃), δ (ppm): 8.36 (br s, 1H), 8.24 (m, 1H), 7.85 (m, 1H), 7.70 (brs, 1H), 7.26-7.14 (m, 4H), 6.78 (brs, 1H), 5.42 (s, 1H), 4.68 (t, 2H, J=5.1), 4.31 (t, 2H, J=5.1), 3.70 (m, 4H), 3.54 (m, 4H), 2.35 (s, 3H), and 2.18 (s, 3H).

MS (ESI): m/z 461.5 (M+H).

### Example 27

### Preparation of Compound 27: 3-{4-[N′-(3-Methyl-benzylidene)-hydrazino]-6-morpholin-4-yl-pyrimidin-2-yl}-propionic acid ethyl ester

Compound 27 was prepared in a similar manner as described in Example 1.

¹H NMR (300 MHz, CDCl₃), δ (ppm): 8.22 (s, 1H); 7.69 (s, 1H); 8.07 (s, 1H); 7.47 (m, 2H); 7.28 (t, J=7.5 Hz, 1H); 7.17 (d, J=7.5 Hz, 1H); 6.23 (s, 1H); 4.13 (q, J=7.2 Hz, 2H); 3.78-3.81 (m, 4H); 3.62-3.65 (m, 4H); 2.98 (t, J=7.2 Hz; 2H); 2.77 (t, J=7.2 Hz, 2H); 2.39 (s, 3H); and 1.24 (t, J=7.2 Hz, 3H).

MS (ESI): m/z 398.2 (M+H).

### Example 28

### Preparation of Compound 28: N-(3-Methyl-benzylidene)-N′-{6-morpholin-4-yl-2-[2-(1-oxy-pyridin-2-yl)-ethoxy]-pyrimidin-4-yl}-hydrazine

To a solution of 4-[6-chloro-2-(2-pyridin-2-yl-ethoxy)-pyrimidin-4-yl]-morpholine (1.61 g, 5.0 mmol) in CH₂Cl₂ (40 ml) was added methanol (10 ml) followed by the addition of MCPBA (70%, 1.43 g, 5.8 mmol) in one portion. The reaction mixture was stirred overnight at room temperature, affording a clear solution. The solution was cast into saturated aqueous NaHCO₃ (35 mL) then the organic phase was separated, washed with 10% aqueous Na₂S₂O₃ (40 mL) and brine (40 mL), and dried (Na₂SO₄), filtered and evaporated in vacuo to give a pure product, 4-{6-chloro-2-[2-(1-oxy-pyridin-2-yl)-ethoxy]-pyrimidin-4-yl}-morpholine as a white solid, (1.46 g, 86.7%).

¹H-NMR (CDCl₃) (ppm), J (Hz): 8.25-8.23 (m, 1H); 7.41-7.7.38 (m, 1H); 7.20-7.16 (m, 2H); 6.14 (s, 1H); 4.71 (t, J=6.0, 2H); 3.77-3.73 (m, 4H); 3.63-3.55 (m, 4H); and 3.40 (t, J=6.0, 2H).

Anhydrous hydrazine (0.640 ml, 20 mmol) was added to a solution of 4-{6-chloro-2-[2-(1-oxy-pyridin-2-yl)-ethoxy]-pyrimidin-4-yl})-morpholine (1.35 g, 4.0 mmol) in dioxane (15 ml) under the nitrogen protection. The obtained mixture was heated at 95-100° C. for 2 h. After it was cooled down, the solvent was evaporated in vacuo until the white solid began to precipitate (to a half the original volume), and then H₂O (15 ml) was added. The resulting precipitate was collected by filtration and washed with water (until the pH was neutral). {6-Morpholin-4-yl-2-[2-(1-oxy-pyridin-2-yl)-ethoxy]-pyrimidin-4-yl}-hydrazine (1.02 g) has been obtained in 76.7% yield.

¹H-NMR (DMSO-d₆) (ppm), J (Hz): 8.25 (bs, 1H); 7.66 (s, 1H); 7.44-7.41 (m, 1H); 7.33-7.25 (m, 2H); 5.59 (s, 1H); 4.46 (t, J=6.0, 2H); 3.64-3.61 (m, 4H); 3.41-3.38 (m, 4); and 3.17 (t, J=6, 2H).

To a solution of {6-morpholin-4-yl-2-[2-(1-oxy-pyridin-2-yl)-ethoxy]-pyrimidin-4-yl}-hydrazine (820 mg, 2.46 mmol) and m-tolualdehyde (97%, 320 mg, 2.58 mmol) in methanol (7 mL) acetic acid (2 drops) was added. The reaction mixture was heated under reflux for 15 min. Upon cooling to room temperature, a precipitating has been formed, and the solid was collected by filtration, washed with little amount of methanol and Et₂O, and dried to afford 950 mg (89%) of N-(3-Methyl-benzylidene)-N′-{6-morpholin-4-yl-2-[2-(1-oxy-pyridin-2-yl)-ethoxy]-pyrimidin-4-yl}-hydrazine as a white solid (m. p. 187-188° C.).

¹H NMR (300 MHz, CDCl₃), δ (ppm): 10.86 (s, 1H); 8.28-8.26 (m, 1H); 7.98 (s, 1H); 7.50-7.43 (m, 3H); 7.33-7.26 (m, 3H); 7.17 (d, J=7.8 Hz, 1H); 6.05 (s, 1H); 4.53 (t, J=6.3 Hz, 2H); 3.68-3.64 (m, 4H); 3.54-3.50 (m, 4H); 3.21 (t, J=6.3, 2H); and 2.33 (s, 3H).

ESMS calcd for C₂₃H₂₆N₆O₃: 434.21; Found: 457.2 (M+Na)⁺.

### Example 29

### Preparation of Compound 29: 1-(2-{4-[N′-(3-Methyl-benzylidene)-hydrazino]-6-morpholin-4-yl-pyrimidin-2-yloxy}-ethyl)-1H-pyridin-2-one

1-(hydroxy-ethyl)-1H-pyridin-2-one (1.5 g, 10.7 mmol) was coupled with 4-(2,6-dichloropyrimidin-4-yl)-morpholine in the presence of sodium hydride in DMF. After addition of water, precipitate was filtered out, washed with water, and dried to afford almost a desired regioisomer (1.7 g, 47%). The obtained regioisomer was refluxed with 3.5 equivalents of hydrazine in dioxane. Water was added to the reaction mixture, and precipitate was formed. The precipitate was collected by filtration, washed 3 times with water, and dried to give a hydrazine derivative (1.7 g, 85%). Condensation with m-tolyl aldehyde afforded title compound (2.1 g, 95%).

¹H NMR (DMSO-d₆): δ 10.90 (s, 1H), 7.98 (s, 1H), 7.62 (dd, J=6.8, 2.1 Hz, 1H), 7.49 (d, J=7.5 Hz, 1H), 7.48 (s, 1H), 7.41 (td, J=7.8, 2.1 Hz, 1H), 7.29 (t, J=7.5 Hz, 1H), 7.17 (d, J=7.8 Hz, 1H), 6.39 (d, J=9.3 Hz, 1H), 6.20 (t, J=6.2 Hz, 1H), 6.05 (s, 1H), 4.43 (t, J=5.1 Hz, 2H), 4.22 (t, J=5.2 Hz, 2H), 3.66 (m, 4H), 3.52 (m, 4H), 2.34 (s, 3H).

ESMS calcd for C₂₃H₂₆N₆O₃: 434.21; Found: 457.2 (M+23)⁺.

### Example 30

### Preparation of Compound 30: N-(3-Iodo-benzylidene)-N′-[6-morpholin-4-yl-2-(2-pyridin-2-yl-ethoxy)-pyrimidin-4-yl]-hydrazine

Compound 30 was prepared in a similar manner as described in Example 29.

¹H NMR (DMSO-d₆): δ 10.97 (s, 1H), 8.51 (d, J=4.5 Hz, 1H), 8.00 (s, 1H), 7.95 (s, 1H), 7.78-7.70 (m, 3H), 7.34 (d, J=7.8 Hz, 1H), 7.26-7.18 (m, 2H), 6.08 (s, 1H), 4.55 (t, J=6.6 Hz, 2H), 3.66 (m, 4H), 3.53 (m, 4H), 3.14 (t, J=6.6 Hz, 2H).

ESMS calcd for C₂₂H₂₃IN₆O₂: 530.09; Found: 531.1 (M+1)⁺.

### Example 31

### Preparation of Compound 31: N-(3-fluoro-benzylidene)-N′-[6-morpholin-4-yl-2-(2-pyridin-2-yl-ethoxy]-pyrimidin-4-yl]-hydrazine

Compound 31 was prepared in a similar manner as described in Example 29.

¹H NMR (DMSO-d₆): δ10.98 (s, 1H), 8.51 (d, J=3.9 Hz, 1H), 8.01 (s, 1H), 7.72 (td, J=7.6, 1.8 Hz, 1H), 7.57 (brd, J=9.9 Hz, 1H), 7.51-7.40 (m, 2H), 7.33 (d, J=7.2 Hz, 1H), 7.24 (dd, J=7.6, 5.2 Hz, 1H), 7.20 (brt, J=7.8 Hz, 1H); 6.11 (s, 1H), 4.54 (t, J=6.8 Hz, 2H), 3.65 (m, 4H), 3.54 (m, 4H), 3.14 (t, J=6.7 Hz, 2H).

ESMS calcd for C₂₂H₂₃FN₆O₂: 422.19; Found: 445.2 (M+23)⁺.

### Example 32

### Preparation of Compound 32: N-(3-chloro-benzylidene)-N′-[6-morpholin-4-yl-2-(2-pyridin-2-yl-ethoxy)-pyrimidin-4-yl]-hydrazine

Compound 32 was prepared in a similar manner as described in Example 29.

¹H NMR (DMSO-d₆): δ 11.00 (s, 1H), 8.51 (d, J=4.5 Hz, 1H), 8.00 (s, 1H), 7.74-7.70 (m, 2H), 7.65 (d, J=6.6 Hz, 1H), 7.45-7.41 (m, 2H), 7.33 (d, J=7.8 Hz, 1H), 7.24 (dd, J=7.8, 4.8 Hz, 1H), 6.09 (s, 1H), 4.54 (t, J=6.6 Hz, 2H), 3.66 (m, 4H), 3.54 (m, 4H), 3.14 (t, J=6.6 Hz, 2H).

ESMS calcd for C₂₂H₂₃CIN₆O₂: 438.16; Found: 461.2 (M+23)⁺.

### Example 33

### Preparation of Compound 33: N-(3-bromo-benzylidene)-N′-[6-morpholin-4-yl-2-(2-pyridin-2-yl-ethoxy)-pyrimidin-4-yl]-hydrazine

Compound 33 was prepared in a similar manner as described in Example 29.

¹H NMR (DMSO-d₆): δ 10.99 (s, 1H), 8.51 (d, J=4.2 Hz, 1H), 7.98 (s, 1H), 7.86 (s, 1H), 7.72 (t, J=8.5 Hz, 1H), 7.71 (d, J=8.1 Hz, 1H), 7.54 (d, J=7.5 Hz, 1H), 7.38-7.32 (m, 2H), 7.24 (dd, J=7.2, 4.8 Hz, 1H), 6.09 (s, 1H), 4.54 (t, J=6.6 Hz, 2H), 3.66 (m, 4H), 3.53 (m, 4H), 3.14 (t, J=6.6 Hz, 2H).

ESMS calcd for C₂₂H₂₃BrN₆O₂: 482.11; Found: 505.10 (M+23)⁺.

### Example 34

### Preparation of Compound 34: 3-{[6-Morpholin-4-yl-2-(2-pyridin-2-yl-ethoxy)-pyrimidin-4-yl]-hydrazonomethyl}-benzoic acid methyl ester

Compound 34 was prepared in a similar manner as described in Example 29.

¹H NMR (DMSO-d₆): δ 11.00 (s, 1H), 8.51 (d, J=5.4 Hz, 1H), 8.12 (s, 1H), 8.10 (s, 1H), 8.06 (d, J=8.1 Hz, 1H), 7.93 (d, J=6.6 Hz, 1H), 7.73 (t, J=7.6 Hz, 1H), 7.57 (t, J=8.0 Hz, 1H), 7.34 (d, J=7.8 Hz, 1H), 7.24 (dd, J=6.0, 4.5 Hz, 1H), 6.07 (s, 1H), 4.55 (t, J=6.4 Hz, 2H), 3.88 (s, 3H), 3.68 (m, 4H), 3.53 (m, 4H), 3.15 (t, J=6.6 Hz, 2H).

ESMS calcd for C₂₄H₂₆N₆O₄: 462.20; Found: 463.3 (M+1)⁺.

### Example 35

### Preparation of Compound 35: 1-(2-{4-[N′-(3-Iodo-benzylidene)-hydrazino]-6-morpholin-4-yl-pyrimidin-2-yloxy}-ethyl)-1H-pyridin-2-one

Compound 35 was prepared in a similar manner as described in Example 29.

¹H NMR (DMSO-d₆): δ 11.02 (s, 1H), 8.00 (s, 1H), 7.93 (s, 1H), 7.75-7.69 (m, 2H), 7.61 (dd, J=7.0, 1.8 Hz, 1H), 7.41 (td, J=7.9, 2.1 Hz, 1H), 7.20 (t, J=8.0 Hz, 1H), 6.38 (d, J=8.4 Hz, 1H), 6.19 (t, J=6.7 Hz, 1H), 6.06 (s, 1H), 4.43 (t, J=5.3 Hz, 2H), 4.22 (t, J=5.3 Hz, 2H), 3.66 (m, 4H), 3.53 (m, 4H), 3.14 (t, J=6.6 Hz, 2H).

ESMS calcd for C₂₂H₂₃IN₆O₃: 546.09; Found: 569.2 (M+23)⁺.

### Example 36

### Preparation of Compound 36: 3-{[6-Morpholin-4-yl-2-(2-pyridin-2-yl-ethoxy)-pyrimidin-4-yl]-hydrazonomethyl}-benzoic acid N-methyl amide

Compound 36 was prepared in a similar manner as described in Example 29.

¹H NMR (DMSO-d₆): δ 11.00 (s, 1H), 8.6 (s, 1H), 8.41 (d. J=5.4 Hz, 1H), 8.12 (s, 1H), 8.11 (s, 1H), 8.0 (d, J=8.1 Hz, 1H), 7.83 (d, J=68 Hz, 1H); 7.73 (t, J=7.2 Hz, 1H), 7.57 (t, J=8.0 Hz, 1H), 7.34 (d, J=7.8 Hz, 1H). 7.34 (dd, J=6.0, 4.5 Hz, 1H), 6.07 (s, 1H), 4.55 (t, J=6.4 Hz, 2H), 3.5-3.0 (m, 7H).

ESMS calcd for C₂₄H₂₇N₇O₃: 461.2; Found: 485.1 (M+Na)⁺.

### Example 37

### Preparation of Compound 37: (3-{[6-Morpholin-4-yl-2-(2-pyridin-2-yl-ethoxy)-pyrimidin-4-yl]-hydrazonomethyl}-phenyl)-methanol

Compound 37 was prepared in a similar manner as described in Example 29.

¹H NMR (DMSO-d₆): δ 10.86 (s, 1H), 8.51 (d, J=3.9 Hz, 1H), 8.03 (s, 1H), 7.73 (td, J=7.8 and 1.8 Hz, 1H), 7.39 (m, 2H), 7.39-7.32 (m, 3H), 7.24 (dd, J=6.3 and 4.8 Hz, 1H), 6.06 (s, 1H), 5.25 (t, J=5.7 Hz, 1H), 4.54 (t, J=6.8 Hz, 2H); 4.53 (d, J=6.5 Hz, 2H), 3.66 (m, 4H), 3.53 (m, 4H), 3.14 (t, J=6.9 Hz, 2H).

ESMS clcd for C₂₃H₂₆N₆O₃: 434.49; Found: 435.2 (M+1)₊.

Compounds 38-41 were prepared by the following method.

4-Carbamimidoyl-butiric acid ethyl ester hydrochloride was prepared following a procedure starting from 4-cyanobutyrate (6.49 g, 43.9 mmol) and coupled with diethyl malonate in the presence of sodium ethylate to afford desired dihydroxypyrimidine (1.27 g, 15%). Treatment of the dihydroxypyrimidine with phosphorus oxychloride gave dichloro-derivative (0.88 g, 60%), which was converted into morpholine derivative (0.89 g, 85%) after reacting with DIPEA and morpholine in THF. The dichloro-derivative was refluxed in dioxane with 4 equivalents of hydrazine to afford a hydrazine derivative (0.52 g, 59%) that was condensed with m-tolyl aldehyde to obtain hydrazone (0.61 g, 88%). The hydrazone was hydrolyzed with KOH in methanol to yield an acid: 4-{4-[N′-(3-Methyl-benzylidene)-hydrazino]-6-morpholin-4-yl-pyrimidin-2-yl}-butyric acid (0.47 g, 82%).

To a solution of the acid, EDC, DMAP, and an appropriate amine in DMF were added. The obtained reaction mixture was stirred overnight at room temperature, and was distributed between dichloromethane and water layers. The dichloromethane layer was washed two times with water, brine, and dried. The obtained amide (70-80% yield) was isolated by column chromatography.

### Example 38

### Preparation of Compound 38: N,N-Diethyl-4-{4-[N″-(3-methyl-benzylidene)-hydrazino]-6-morpholin-4-yl-pyrimidin-2-yl}-butyramide

¹H NMR (CDCl₃): δ 8.38 (brs, 1H), 7.71 (s, 1H), 7.47 (m, 2H), 7.31-7.26 (m, 2H), 7.17 (d, J=7.5 Hz, 1H), 6.24 (s, 1H), 3.78 (m, 4H), 3.66 (m, 4H), 3.37 (q, J=7.2 Hz, 2H), 3.30 (q, J=7.2 Hz, 2H), 2.67 (t, J=7.4 Hz, 2H), 2.39 (m, 4H), 2.13 (qv, J=7.4 Hz, 2H), 1.13 (t, J=7.4 Hz, 3H), 1.11 (t, J=7.4 Hz, 3H).

ESMS calcd for C₂₄H₃₄N₆O₂: 438.27; Found: 439.30 (M+1)⁺.

### Example 39

### Preparation of Compound 39: 4-{4-[N′-(3-Methyl-benzylidene)-hydrazino]-6-morpholin-4-yl-pyrimidin-2-yl}-1-(4-methyl-piperazin-1-yl)-butan-1-one

¹H NMR (CDCl₃): δ 8.36 (brs, 1H), 7.71 (s, 1H), 7.46 (m, 2H), 7.31-7.26 (m, 2H), 7.17 (d, J=7.8 Hz, 1H), 6.25 (s, 1H), 3.80 (m, 4H), 3.65 (m, 6H), 3.46 (t, J=4.9 Hz, 2H), 2.67 (t, J=7.4 Hz, 2H), 2.42-2.34 (m, 8H), 2.30 (s, 3H), 2.11 (qv, J=7.5 Hz, 2H).

ESMS calcd for C₂₅H₃₅N₇O₂: 465.29; Found: 466.30 (M+1)⁺.

### Example 40

### Preparation of Compound 40: 4-{4-[N′-(3-Methyl-benzylidene)-hydrazino]-6-morpholin-4-yl-pyrimidin-2-yl}-N-pyridin-4-ylmethyl-butyramide

¹H NMR (CDCl₃): δ8.59 (brs, 1H), 7.92 (s, 1H), 7.60 (m, 2H), 7.37 (m, 2H), 7.22-7.11 (m, 4H), 7.00 (m, 1H), 6.15 (s, 1H), 4.36 (d, J=5.7 Hz, 2H), 3.68 (m, 4H), 3.53 (m, 4H), 2.62 (t, J=7.4 Hz, 2H), 2.31 (s, 3H), 2.25 (t, J=6.9 Hz, 2H), 2.05 (qv, J=6.8 Hz, 2H).

ESMS calcd for C₂₆H₃₁N₇O₂: 473.25; Found: 474.30 (M+1)⁺.

### Example 41

### Preparation of Compound 41: 4-(4-[N′-(3-Methyl-benzylidene)-hydrazino]-6-morpholin-4-yl-pyrimidin-2-yl)-N-pyridin-4-yl-butyramide

¹H NMR (CDCl₃): δ 9.43 (s, 1H), 8.68 (brs, 1H), 8.43 (d, J=4.8 Hz, 2H), 7.75 (s, 1H), 7.51 (d, J=5.4 Hz, 2H), 7.44 (m, 2H), 7.27 (t, J=7.2 Hz, 1H), 7.16 (d, J=6.9 Hz, 1H), 6.23 (s, 1H), 3.77 (m, 4H), 3.64 (m, 4H), 2.72 (t, J=6.9 Hz, 2H), 2.46 (t, J=6.9 Hz, 2H), 2.37 (s, 3H), 2.15 (qv, J=6.9 Hz, 2H).

ESMS clcd for C₂₅H₂₉N₇O₂: 459.24; Found: 460.30 (M+1)⁺.

### Example 42

### Preparation of Compound 42: 2-{4-[N′-(3-Methyl-benzylidene)-hydrazino]-6-morpholin-4-yl-pyrimidin-2-yloxy}-1-pyridin-2-yl-ethanol

Compound 42 was synthesized by a similar manner as described in Example 28. The following analytical data were obtained:

¹H NMR (DMSO-d₆): δ 10.82 (s, 1H), 8.52 (d, J=4.2 Hz, 1H), 8.00 (s, 1H), 7.82 (t, J=8.1 Hz, 1H), 7.57 (d, J=7.8 Hz, 1H), 7.48 (d, J=8.5 Hz, 1H), 7.47 (s, 1H), 7.31 (m, 2H), 7.17 (d, J=7.8 Hz, 1H), 6.05 (s, 1H), 5.77 (d, J=5.4 Hz, 1H), 4.93 (m, 1H), 4.52 (dd, J=10.8 and 3.6 Hz, 1H), 4.29 (dd, J=10.7 and 7.0 Hz, 1H), 3.66 (m, 4H), 3.54 (m, 4H), 2.33 (s, 3H); ESMS clcd for C₂₃H₂₆IN₆O₃: 434.21; Found: 435.3 (M+1)⁺.

### Example 43

### Preparation of Compound 43: 6-(2-{4-[N′-(3-Methyl-benzylidene)-hydrazino]-6-morpholin-yl-pyrimidin-2-yloxy}-ethyl)-pyridin-3-ol

Compound 43 was synthesized by a similar manner as described in Example 28. The following analytical data were obtained:

¹H-NMR (DMSO-d₆) δ (ppm), 10.85 (s, 1H), 9.69 (s, 1H), 8.07 (s, 1H), 8.00 (s, 1H), 7.49 (s, 2H), 7.30 (t, J=7.5 Hz, 1H), 7.21-7.12 (m, 3H), 6.07 (s, 1H), 4.47 (t, J=7.5 Hz, 2H), 3.67-3.65 (m, 4H), 3.54-3.53 (m, 4H), 3.03 (t, J=7.5 Hz, 2H), 2.34 (s, 3H);

ESMS clcd for C₂₃H₂₆N₆O₃: 434.21; Found: 457.2 (M+Na)⁺.

### Example 44

### Preparation of Compound 44: 6-(2-{4-[N′-(3-Hydroxymethyl-benzylidene)-hydrazino]-6-morpholin-4-yl-pyrimidin-2-yloxy}-ethyl)-pyridin-3-ol

Compound 44 was synthesized by a similar manner as described in Example 28. The following analytical data were obtained:

¹H-NMR (DMSO-d₆) δ (ppm), 10.85 (s, 1H), 9.68 (s, 1H), 8.05-8.03 (m, 2H), 7.57 (s, 2H), 7.38-7.31 (m, 2H), 7.15-7.09 (m, 2H), 6.05 (s, 1H), 4.53-4.51 (m, 2H), 4.46 (t, J=7.5 Hz, 2H), 3.69-3.62 (m, 4H), 3.52-3.48 (m, 4H), 3.02 (t, J=7.5 Hz, 2H);

ESMS clcd for C₂₃H₂₆N₆O₄: 450.20; Found: 473.2 (M+Na)⁺.

Inhibitory Activity of Exemplary Compounds on Osteoclast Formation

### Example 45

Materials and Methods:

Human peripheral blood mononuclear cells (PBMC) were isolated from healthy donor blood. The cells were seeded in multi-well plates at 7.5×10⁵ cells/ml in RPMI 1640 medium including 10% FBS. Osteoclast formation was induced with 20 ng/ml of recombinant human receptor activator of NF-kB-ligand (RANKL) and 10 ng/ml of human M-CSF in the presence of various doses of test compounds. After 48 hours of culture, RANKL and M-CSF was replenished and further cultured for 2 days. Then, the cultured cells were stained for tartrate-resistant acid phosphatase (TRAP). Osteoclasts were identified as TRAP-positive cells with more than 3 nuclei. Total cell viability was assessed by CCK-8 assay (Dojindo, Gaithersburg, Md.) with 24 hour incubation.

Results:

The tested compounds of this invention significantly reduced osteoclast formation as compared to two positive controls (Tamoxifen and 17β-estradiol). The obtained IC50 values (compound concentration required for 50% inhibition of osteoclast formation) and CC50 values (compound concentration required for 50% inhibition of cell viability) are shown in Table 1.

TABLE 1
IC50 and CC50 values for Osteoclast Formation and Cell Viability

| Compound | IC50 | CC50 |
|---|---|---|
| No. | (nM) | (nM) |
| 12 | 15 | 3250 |
| 28 | 70 | >1000 |
| 42 | 8 | >1000 |
| 43 | 12 | >1000 |
| 44 | 17 | >1000 |
| Tamoxifen | 474 | |
| 17β-estradiol | 78 | |

### Example 46

### Preparation of Compound 101: N-(1H-indol-3-ylmethylene)-N′-[4-morpholin-4-yl-6-(2-pyridin-2-yl-ethoxy)-[1,3,5]triazin-2-yl]-hydrazine

Cyanuric chloride (13.66 g, 74 mmol) was dissolved in methylene chloride (100 mL) at −78° C., followed by the addition of diisopropylethylamine (12.9 mL, 74 mmol). The reaction mixture was stirred for 5 minutes. Morpholine (6.46 mL, 74 mmol) was added dropwise into the reaction mixture in 10 min. The resulting white precipitate was filtered, washed with water, and dried to afford the desired intermediate in quantitative yield (17 g, 100%).

2-(2-Hydroxyethyl)pyridine (2 g, 16.2 mmol) was dissolved in THF (20 mL) at 0° C. 6.6 mL of 2.5 M n-butyl lithium (16.2 mmol) was added into the pyridine solution dropwise in 5 min. The resulting solution was then added dropwise via cannula to a triazine dichloride solution (3.8 g, 16.2 mmol, in THF) at −78° C. The reaction was allowed to warm to room temperature for overnight to yield the triazine monochloride intermediate (2.8 g, 54%) as a white powder.

Hydrazine (0.5 mL, 15.5 mmol) was dissolved in 10 mL ethanol at room temperature. The triazine monochloride intermediate (1 g, 3.11 mmol) was added to a solution of ethanol (20 mL) and heated to 60° C. before adding into the hydrazine solution. After stirring for 30 ml, white crystals precipitated, which were then filtered, washed with water and air dried to yield the triazine hydrazine intermediate (781 mg, 78%) as a white powder.

Indole-3-aldehyde (1.05 g, 7.25 mmol) and the triazine hydrazine intermediate (2.3 g, 7.25 mmol) were added to 30 mL of methanol at room temperature. 5 mL of acetic acid was added to the reaction mixture and was refluxed for 5 min. Upon cooling, a white precipitate was formed, which was filtered and washed with water to yield Compound 101 as a white powder (1.7 g, 52%).

¹H NMR (CDCl₃), δ (ppm): 3.28 (t, J=6.9, 2H); 3.7 (broad s, 4H); 3.86 (broad s, 4H); 4.73 (broad t, 2H); 7.14-7.24 (m, 2H); 7.27-7.30 (m, 3H); 7.37 (d, J=8.1, 1H); 7.45 (d, J=2.4, 1H); 7.59 (t, J=7.5, 1H); 8.14 (s, 1H); 8.42 (d, J=7.8, 1H); 8.49 (s, 1H); and 8.56 (d, J=8.5, 1H).

MS (ESI): m/z 445.2 (M+H).

### Example 47

### Preparation of Compound 102: 2,3-dimethyl-1H-indol-5-yl)-[4-morpholin-4-yl-6-(2-pyridin-2-yl-ethoxy)-[1,3,5]triazin-2-yl]-amine

To a solution of cyanuric chloride (0.922 g, 5.00 mmol, 1.00 equiv.) in 15 mL CH₂Cl₂ at 0° C. was added slowly DIPEA (1.422 g, 11.00 mmol, 2.20 equiv.) during a period of 10 minutes. Ice bath was removed, and 2-(2-hydroxyethyl)pyridine (0.677 g, 5.50 mmol, 1.10 equiv.) was added, and the reaction mixture was stirred at room temperature for 15 minutes. 5-Amino-2,3-dimethylindole (0.641 g, 4.00 mmol, 0.80 equiv.) was then added, and stirred for 4 hours at room temperature. A light brown solid precipitated out after 10 mL of water was added to the reaction mixture and stirred for about 10 minutes; The light brown solid was collected by filtration, washed with 2×10 mL water, 5 mL EtOAc and dried (1.50 g, 3.80 mmol, 95%). This solid was then added to a solution of morpholine (0.827 g, 9.5 mmol, 2.50 equiv.) in 30 mL THF, and stirred at 60° C. for 4 hours. Usual workup and flash chromatography purification gave Compound 102 as an off-white solid (1.30 g, 2.92 mmol, 77%).

¹H NMR (300 MHz, DMSO-d₆), δ ppm: 10.50 (s, 1H); 9.29 (br s, 1H); 8.51 (d, J=4.8 Hz, 1H); 7.70-7.79 (m, 2H); 7.22-7.34 (m, 2H); 7.10 (s, 2H); 4.63 (t, J=6.9 Hz, 2H); 3.71 (brs, 4H); 3.63 (br s, 4H); 3.16 (t, J=6.9 Hz, 2H); 2.78 (s, 3H), 2.07 (br s, 3H); MS (ESI): m/z 446.2 (M+H)⁺.

### Example 48

### Preparation of Compound 103: N-(1H-indol-3-Ylmethylene)-N′-[4-morpholin-4-yl-6-(2-pyridin-3-yl-ethoxy)-[1,3,5]triazin-2-yl]-hydrazine

Compound 103 was prepared in a similar manner as described in Example 46.

¹H NMR (300 MHz, CDCl₃), δ ppm: 9.10 (br s, 1H); 8.55 (d, J=1.8 Hz, 1H); 8.47-8.49 (m, 2H); 8.34-8.41 (m, 1H); 8.07 (s, 1H); 7.60 (dt, J=1.8 Hz, 7.5 Hz, 1H); 7.34-7.39 (m, 2H); 7.14-7.25 (m, 3H); 4.58 (br s, 2H); 3.86 (br s, 4H); 3.75 (br s, 4H); 3.09 (t, J=7.2 Hz, 1H); MS (ESI): m/z 445.1 (M+H)⁺.

### Example 49

### Preparation of Compound 104: N-(3-Methoxy-benzylidene)-N′-[4-morpholin-4-yl-6-(2-pyridin-2-yl-ethoxy)-[1,3,5]triazin-2-yl]-hydrazine

Compound 104 was prepared in a similar manner as described in Example 46.

¹H NMR (300 MHz, DMSO-d₆), δ ppm: 11.19 (s, 1H); 8.52 (dd, J=3.9 Hz, 0.9 Hz, 1H); 8.07 (s, 1H); 7.73 (m, 1H); 7.19-7.36 (m, 4H); 6.95 (dd, J=7.8 Hz, 2.4 Hz, 1H); 4.64 (t, J=6.3 Hz, 2H); 3.64-3.78 (m, 11H); 3.17 (t, J=6.3 Hz, 2H); MS (ESI): m/z 436.2 (M+H)⁺.

### Example 50

### Preparation of Compound 105: N-(3-methyl-benzylidene)-N′-[4-morpholin-4-yl-6-(2-pyridin-2-yl-ethoxy)-[1,3,5]triazin-2-yl]-hydrazine

Compound 105 was prepared in a similar manner as described in Example 46.

¹H NMR (300 MHz, DMSO-d₆), δ ppm: 11.14 (s, 1H); 8.52 (dd, J=3.9 Hz, 0.9 Hz, 1H); 8.07 (s, 1H); 7.73 (m, 1H); 7.17-7.45 (m, 6H); 4.64 (t, J=6.3 Hz, 2H); 3.63-3.73 (m, 8H); 3.17 (t, J=6.3 Hz, 2H); 2.33 (s, 3H); MS (ESI): m/z 420.2 (M+H)⁺.

### Example 51

### Preparation of Compound 106: 4-{4-[N′-(1H-indol-3-ylmethylene)-hydrazino]-6-morpholin-4-yl-[1,3,5]triazin-2-yl}-butan-1-ol

Compound 106 was prepared in a similar manner as described in Example 52.

¹H NMR (300 MHz, CDCl₃+ DMSO-d₆, 8.1), δ ppm: 10.16 (br s, 1H); 9.17 (br s, 1H); 8.37-8.47 (m, 1H); 8.21 (s, 1H); 7.36-7.47 (m, 3H); 7.17-7.26 (m, 2H); 3.93 (br s, 4H); 3.77 (br s, 4H); 3.65 (t, J=6.3 Hz, 2H); 2.62 (br s, 2H); 1.84-1.92 (m, 2H); 1.62-1.71 (m, 2H); MS (ESI): m/z 396.2 (M+H)⁺.

### Example 52

### Preparation of Compound 107: N-{4-[3-(3,4-dimethoxy-phenyl)-propyl]-6-morpholin-4-yl-1,3,5-triazin-2-yl}-N′-[1-(1H-indol-3-yl)-meth-(E)-ylidene]-hydrazine

To a solution of 3-(3,4-dimethoxyphenyl)-propyl iodide (1.224 g, 4.00 mmol, 1.00 equiv.) in 20 mL dry THF was added highly active zinc (suspension in THF, Rieke metal from Aldrich, 5.2 mL 0.05 g/mL, 4.00 mmol, 1.00 equiv.). The mixture was stirred at room temperature overnight. 2,4-dichloro-6-morpholin-4-yl-1,3,5-triazine (0.936 g, 4.0 mmol, 1.00 equiv.) and trans-benzyl-(chloro)-bis-(triphenylphosphine)palladium(II) (0.03 g, 0.04 mmol, 0.01 equiv.) were added, and the reaction mixture was stirred at room temperature for 8 hours. Usual workup and flash chromatography purification gave 4-chloro-2-[3-(3,4-dimethoxyphenyl)propyl]-6-morpholin-4-yl-1,3,5-triazine as a light yellow solid which was treated with hydrazine following the typical procedure to yield {4-[3-(3,4-Dimethoxy-phenyl)-propyl]-6-morpholin-4-yl-1,3,5-triazin-2-yl}-hydrazine as a white solid (0.85 g, 2.27 mmol, 57%). MS (ESI): m/z 375.2 (M+H)⁺.

A mixture of {4-[3-(3,4-dimethoxy-phenyl)-propyl]-6-morpholin-4-yl-1,3,5-triazin-2-yl}-hydrazine (0.75 g, 2.00 mmol, 1.00 equiv.), indole-3-carboxaldehyde (0.29 g, 2.00 mmol, 1.00 equiv.), and AcOH (80 mg, cat.) in 15 mL MeOH was stirred at 75° C. for 4 hours. Solvent was removed and the residue was subjected to flash chromatography purification and crystallization in MeOH to yield Compound 107 as an off-white solid (0.72 g, 1.44 mmol, 72%).

¹H NMR (300 MHz, CDCl₃), δ ppm: 8.57 (br s, 1H); 8.45 (br s, 1H); 8.29-8.32 (m, 1H); 8.00 (s, 1H); 7.39-7.43 (m, 2H); 7.23-7.34 (m, 2H); 6.74-6.80 (m, 3H); 6.30 (s, 1H); 3.86 (s, 3H); 3.85 (s, 3H); 3.78-3.84 (m, 4H); 3.67-3.70 (m, 4H); 2.63-2.71 (m, 4H), 2.03-2.13 (m, 2H); MS (ESI): m/z 502.2 (M+H)⁺.

### Example 53

### Preparation of Compound 108: N-{4-[2-(2,2-Dimethyl-[1,3]-dioxolan-4-yl)-ethoxy]-6-morpholin-4-yl-[1,3,5]triazin-2-yl}-N′-(1H-indol-3-ylmethylene)-hydrazine

Compound 108 was prepared in a similar manner as described in Example 46.

¹H NMR (300 MHz, CD₃Cl) δ (ppm): 8.50 (s, 1H), 8.42 (d, J=8.4 Hz, 1H), 8.24 (s, 1H), 8.09 (s, 1H), 7.44 (d, J=3.0 Hz, 1H), 7.38 (d, 1H, J=7.2 Hz), 7.20-7.26 (m, 2H), 4.55 (br, 2H), 4.28 (d, J=7.4 Hz, 1H) 3.84 (m, 4H), 3.71 (m, 4H), 3.60 (t, J=7.4 Hz, 2H), 2.03 (m, 2H), 1.42 (s, 3H), 1.35 (s, 3H). MS (ESI): m/z 468.3 (M+H)⁺.

### Example 54

### Preparation of Compound 109: N-[4-(4,5-dihydro-oxazol-2-ylmethoxy)-6-morpholin-4-yl-[1,3,5]triazin-2-yl]-N′-(1H-indol-3-ylmethylene)-hydrazine

Compound 109 was prepared in a similar manner as described in Example 46.

¹H NMR (300 MHz, DMSO-d₆) δ (ppm): 11.40 (s, 1H), 10.91 (s, 1H), 8.32-8.28 (m, 2H), 7.68 (bs, 1H), 7.40-7.37 (m, 1H), 7.21-7.05 (m, 2H), 4.80-4.66 (m, 4H), 3.75-3.55 (m, 8H), 3.15 (s, 2H); MS (ESI): m/z 423.1.

### Example 55

### Preparation of Compound 110: {4-[N′-(1H-indol-3-ylmethylene)-hydrazino]-6-morpholin-4-yl-[1,3,5]triazin-2-yloxy}-acetic acid ethyl ester

Compound 110 was prepared in a similar manner as described in Example 46.

¹H NMR (300 MHz, DMSO-d₆) δ (ppm): 8.62-8.60 (m, 1H), 8.42 (d, 1H, J=9.0 Hz), 8.09 (s, 1H), 7.45 (bs, 1H), 7.39-7.36 (m, 1H), 7.28-7.20 (m, 3H), 4.84 (s, 0.2H), 4.27-4.19 (m, 2H), 3.80-3.65 (m, 8H), 1.25 (t, 3H, J=7.2 Hz); MS (ESI): m/z 426.1.

### Example 56

### Preparation of Compound 111: N-(2-hydroxy-ethyl)-2-{4-[N′-(1H-indol-3-ylmethylene)-hydrazino]-6-morpholin-4-yl-[1,3,5]triazin-2-yloxy}-acetamide

Compound III was prepared in a similar manner as described in Example 46.

¹H NMR (DMSO-d₆) δ (ppm): 11.40 (s, 1H), 10.92 (s, 1H), 8.32-8.28 (m, 2H), 8.00-7.93 (m, 1H), 7.69 (bs, 1H), 7.40-7.37 (m, 1H), 7.21-7.05 (m, 2H), 4.75-4.60 (m, 4H), 3.75-3.55 (m, 8H), 3.20-3.10 (m, 2H); MS (ESI): m/z 441.1.

### Example 57

### Preparation of Compound 112: 4-[4-(2,3-Dimethyl-1H-indol-5-ylamino)-6-morpholin-4-yl-[1,3,5]triazin-2-yloxy]-benzonitrile

Compound 112 was prepared in a similar manner as described in Example 47.

¹H-NMR (300 MHz, DMSO-d₆), δ (ppm): 1.93 (s, 1H), 2.08 (s, 2H), 2.27 (s, 3H), 3.74-3.27 (m, 8H), 6.99 (s, 1H), 7.09 (s, 1H), 7.46 (d, J=8.7 Hz), 7.79 (s, 1H), 7.91 (d, J=8.7 Hz), 9.46 (s, 1H), 10.51 (s, 1H). MS (ESI): m/z 441.2 (M+H)⁺.

### Example 58

### Preparation of Compound 113: Dibenzofuran-3-yl-{4-[2-(3,4-dimethoxy-phenyl)-ethoxy]-6-morpholin-4-yl-[1,3,5]triazin-2-yl}-amine

Compound 113 was prepared in a similar manner as described in Example 47.

¹H NMR (DMSO) 2.94 (t, 2H, J=6.9), 3.64-3.70 (m, 14H), 4.46 (t, 2H, J=6.9), 6.79 (q, 2H, J=6.9), 6.90 (s, 1H), 7.33 (m, 1H), 7.49 (t, 1H, J=8.4), 7.61 (m, 2H), 7.85 (br s, 1H), 8.49 (s, 1H), 9.70 (s, 1H). ESMS: calculated for C₂₉H₂₉N₅O₅: 527.5; found: 528.2 (M+H).

### Example 59

### Preparation of Compound 114: Dibenzofuran-3-yl-{4-morpholin-4-yl-6-[2-(pyridin-3-yloxy)-ethoxy]-[1,3,5]triazin-2-yl}-amine

Compound 114 was prepared in a similar manner as described in Example 47.

¹H NMR (CDCl₃): δ 8.22 (s, 1H), 7.57-7.20 (m, 8H), 6.8-7.1 (m, 3H), 4.60 (m, 2H), 4.44 (m, 2H), 3.8-3.5 (m, 2H); ESMS clcd for C₂₉H₂₉N₅O₅: 527.22; Found: 528.2 (M+1)⁺.

### Example 60

### Preparation of Compound 115: (2,3-Dimethyl-1H-indol-6-yl)-{4-morpholin-4-yl-6-[2-(pyridin-3-yloxy)-ethoxy]-[1,3,5]triazin-2-yl}-amine

Compound 115 was prepared in a similar manner as described in Example 47.

¹H NMR (CDCl₃): δ 8.33 (s, 1H), 8.23 (t, J=2.6 Hz, 1H), 7.72 (m, 2H), 7.20 m, 2H), 7.17 (s, 1H), 7.13 (m, 1H), 6.95 (s, 1H), 4.69 (t, J=4.9 Hz, 2H), 4.34 (t, J=4.9 Hz, 2H), 3.84 (m, 4H), 3.71 (m, 4H), 2.36 (s, 3H), 2.17 (s, 3H); ESMS clcd for C₂₄H₂₇IN₇O₃: 461.22; Found: 462.2 (M+1)⁺.

### Example 61

### Preparation of Compound 116: N-(1H-indol-3-ylmethylene)-N′-[4-morpholin-4-yl-6-(2-pyridin-2-yl-ethoxy)-[1,3,5]triazin-2-yl]-hydrazine

Cyanuric chloride (13.66 g, 74 mmol) was dissolved in methylene chloride (100 mL) at −78° C., followed by the addition of diisopropylethylamine (12.9 mL, 74 mmol). The reaction mixture was stirred for 5 minutes. Morpholine (6.46 mL, 74 mmol) was added dropwise into the reaction mixture in 10 min. The resulting white precipitate was filtered, washed with water, and dried to afford the desired intermediate in quantitative yield (17 g, 100%).

2-(2-Hydroxyethyl)pyridine (2 g, 16.2 mmol) was dissolved in THF (20 mL) at 0° C. 6.5 mL of 2.5 M n-butyl lithium (16.2 mmol) was added into the pyridine solution dropwise in 5 min. The resulting solution was then added dropwise via cannula to a triazine dichloride solution (3.8 g, 16.2 mmol, in THF) at −78° C. The reaction was allowed to warm to room temperature for overnight to yield the triazine monochloride intermediate (2.8 g, 54%) as a white powder.

Hydrazine (0.5 mL, 15.5 mmol) was dissolved in 10 mL ethanol at room temperature. The triazine monochloride intermediate (1 g, 3.11 mmol) was added to a solution of ethanol (20 mL) and heated to 60° C. before adding into the hydrazine solution. After stirring for 30 min, white crystals precipitated, which were then filtered, washed with water and air dried to yield the triazine hydrazine intermediate (781 mg, 78%) as a white powder.

Indole-3-aldehyde (1.05 g, 7.25 mmol) and the triazine hydrazine intermediate (2.3 g, 7.2.5 mmol) were added to 30 mL of methanol at room temperature. 5 mL of acetic acid was added to the reaction mixture and was refluxed for 5 min. Upon cooling, a white precipitate was formed, which was filtered and washed with water to yield Compound 116 as a white powder (1.7 g, 52%).

¹H NMR (CDCl₃), δ (ppm): 3.28 (t, J=6.9, 2H); 3.7 (broad s, 4H); 3.86 (broad s, 4H); 4.73 (broad t, 2H); 7.14-7.24 (m, 2H); 7.27-7.30 (m, 3H); 7.37 (d, J=8.1, 1H); 7.45 (d, J=2.4, 1H); 7.59 (t, J=7.5, 1H); 8.14 (s, 1H); 8.42 (d, J=7.8, 1H); 8.49 (s, 1H); and 8.56 (d, J=8.5, 1H).

MS (ESI): m/z 445.2 (M+H)

Inhibitory Activity of Exemplary Compounds on Osteoclast Formation

### Example 62

### Materials and Methods

Human peripheral blood mononuclear cells (PBMC) were isolated from healthy donor blood. The cells were seeded in multi-well plates at 7.5×10⁵ cells/ml in RPMI 1640 medium including 10% FBS. Osteoclast formation was induced with 20 ng/ml of recombinant human receptor activator of NF-kB-ligand (RANKL) and 10 ng/ml of human M-CSF in the presence of various doses of test compounds. After 48 hours of culture, RANKL and M-CSF was replenished and further cultured for 2 days. Then, the cultured cells were stained for tartrate-resistant acid phosphatase (TRAP). Osteoclasts were identified as TRAP-positive cells with more than 3 nuclei. Total cell viability was assessed by CCK-8 assay (Dojindo, Gaithersburg, Md.) with 24 hour incubation.

Results:

The tested compounds of this invention significantly reduced osteoclast formation as compared to two positive controls (Tamoxifen and 17β-estradiol). The obtained IC50 values (compound concentration required for 50% inhibition of osteoclast formation) and CC50 values (compound concentration required for 50% inhibition of cell viability) are shown in Table 2.

TABLE 2
IC50 and IC50 values for Osteoclast Formation and Cell Viability

| Compound | | |
|---|---|---|
| No. | IC50 (nM) | CC50 (nM) |
| 102 | 3 | >1000 |
| 113 | 12 | >1000 |
| 114 | 24 | >1000 |
| 115 | 16 | >1000 |
| 116 | 35 | >10,000 |
| Tamoxifen | 474 | |
| 17β-estradiol | 78 | |

### Example 63

### Preparation of {6-morpholin-4-yl-2-[2-(pyridin-2-yloxy)-ethoxy]-9H-purin-8-yl}-m-tolyl-amine

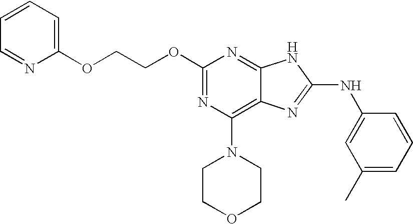

The title compound was synthesized by one of the following two methods:

### Method A:

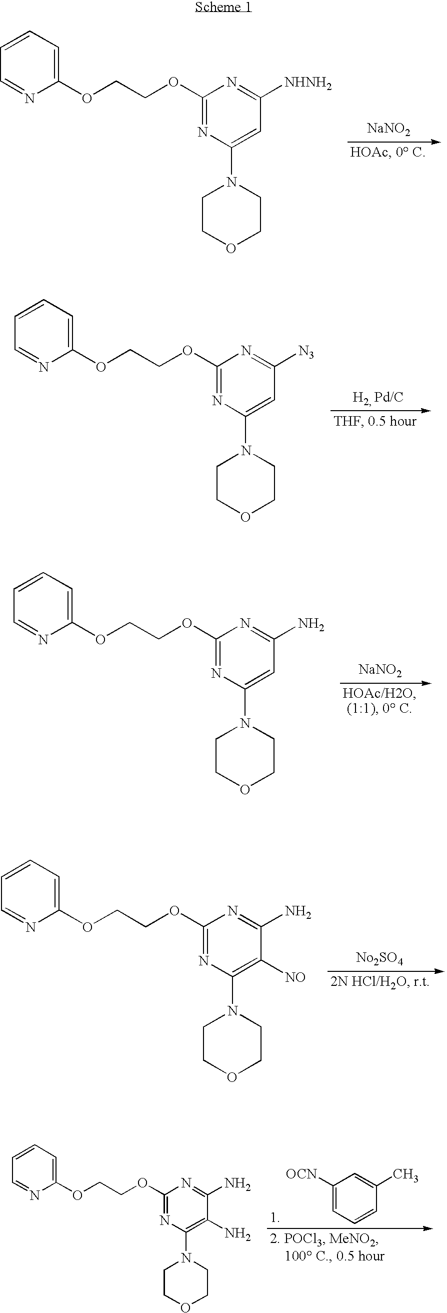

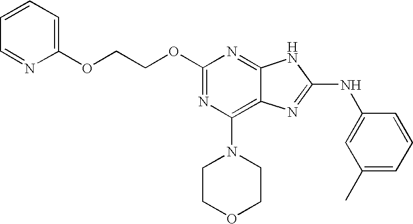

As shown in Scheme 1 above, to a solution of 2-[2-(pyridin-2-yloxy)-ethoxy]-6-hydrazino-4-morphlinopyrimidine (4.98 g, 15.00 mmol, 1.00 equiv.) in 40 mL HOAc was added NaNO₂ (1.553 g, 22.50 mmol, 1.50 equiv.) in six portions over a period of 1 hour. The reaction mixture was stirred at room temperature for 1 hour, and subjected to usual workup to yield 6-azido-2-[2-(pyridin-2-yloxy)-ethoxy]-4-morphlinopyrimidine as green viscous oil (5.0 g, 14.57 mmol, 97% yield). This oil was dissolved in 80 mL THF, and subjected to hydrogenation in the presence of 10% Pd on carbon (0.775 g of 10% Pd/C, 0.73 mmol, 0.05 equiv.) to yield 6-amino-2-[2-(pyridin-2-yloxy)-ethoxy]-4-morphlinopyrimidine as light yellow solid (4.25 g, 13.4 mmol, 89% total yield).

¹H NMR (300 MHz, CDCl₃), δ (ppm): 8.11-8.14 (m, 1H); 7.57 (dd, J=6.9 Hz, 2.1 Hz, 1H); 7.54 (dd, J=5.4 Hz, 2.1 Hz, 1H); 8.00 (s, 1H); 6.87-6.75 (m, 2H); 5.23 (s, 1H); 4.93 (br s, 2H); 4.62 (s, 4H); 3.72-3.75 (m, 4H); 3.48-3.52 (m, 4H).

6-Amino-2-[2-(pyridin-2-yloxy)-ethoxy]-4-morphlinopyrimidine (1.90 g, 6.00 mmol, 1.0 equiv.) was dissolved in 8 mL HOAc, and 8 mL H₂O was added. The solution was cooled to 0° C., and NaNO₂ (0.414 g, 6.00 mmol, 1.0 equiv.) was added. The reaction mixture was stirred at 0° C. for 1 hour. Water (20 mL) was added to dilute the slurry, and the solid was collected by filtration, washed with water, EtOAc (2 mL), then dried to yield 6-amino-2-[2-(pyridin-2-yloxy)-ethoxy]-4-morphlino-5-nitroso-pyrimidine (1.47 g, 4.25 mmol, 85% yield) as blue solid. The nitroso compound (1.385 g, 4.00 mmol, 1.0 equiv.) was treated with 5 mL water and enough 2 N HCl so that a clear dark blue solution was formed. Na₂S₂O₄ (2.79 g, 16.00 mmol, 4.0 equiv.) was added in three portions, and the solution was stirred at room temperature for 1 hour. The resulting clear yellow solution was carefully neutralized with cold 2 M NaOH solution, and subjected to EtOAc extraction. 5,6-Diamino-2-[2-(pyridin-2-yloxy)-ethoxy]-4-morphlinopyrimidine (0.80 g, 2.41 mmol, 60%) was obtained as light yellow solid after usual workup.

¹H NMR (300 MHz, CDCl₃), δ (ppm): 8.12-8.14 (m, 1H); 7.52-7.58 (m, 1H); 6.83-6.87 (m, 1H); 6.75-6.78 (m, 1H); 4.57-4.65 (m, 6H); 3.79-3.83 (m, 4H); 3.22-3.26 (m, 4H); 2.71 (br s, 2H).

ESMS calcd. for C₁₅H₂₁N₆O₃ 332.1; Found: 333.1 (M+H)⁺.

5,6-Diamino-2-[2-(pyridin-2-yloxy)-ethoxy]-4-morphlinopyrimidine (0.332 g, 1.00 mmol, 1.00 equiv.) and m-tolyl isocyanate (0.133 g, 1.00 mmol, 1.00 equiv.) were mixed in 10 mL THF and stirred at room temperature for 15 hours. THF was removed, and the residue was treated with POCl₃ in 2 mL CH₃NO₂ at 100° C. for 30 minutes. The reaction mixture was neutralized with 2N NaOH solution at 0° C., and subjected to EtOAc extraction. The organic solution was dried over MgSO₄, filtered through a plug of silica gel, concentrated to around 2 mL, and cooled to 0° C., resulting in formation of the titled compound as off-white crystal which was collected by filtration, washed with EtOAc, and dried (0.095 g, 0.212 mmol, 21.2% yield).

¹H NMR (300 MHz, DMSO-d₆), δ (ppm): 11.70 (s, 1H); 9.10 (s, 1H); 8.16-8.18 (m, 1H); 7.69-7.75 (m, 1H); 7.43 (s, 1H); 7.35 (d, J=8.1 Hz, 1H); 7.14 (t, J=7.8 Hz, 1H); 6.97-7.01 (m, 1H); 6.85 (d, J=7.8 Hz, 1H); 6.71 (d, J=7.8 Hz, 1H); 4.52-4.57 (m, 4H); 4.09 (br s, 4H); 3.69-3.72 (m, 4H); 2.27 (s, 3H).

ESMS calcd. for C₂₃H₂₆N₇O₃: 447.2; Found: 448.2 (M+H)⁺.

### Method B:

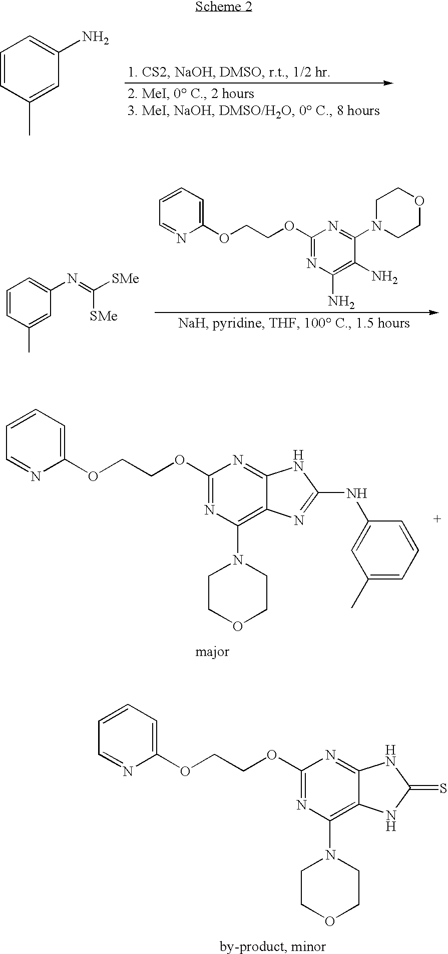

As shown in Scheme 2 above, 5,6-diamino-2-[2-(pyridin-2-yloxy)-ethoxy]-4-morphlinopyrimidine (0.166 g, 0.5 mmol, 1.00 equiv.), dimethyl N-(m-tolyl)-dithioiminocarbonate (0.106 g, 0.5 mmol, 1.00 equiv., prepared from m-toluidine, CS₂, NaOH and MeI), pyridine (0.2 mL), and THF (5 mL) were mixed in a sealed tube. NaH (0.12 g 600% in oil, 3 mmol, 6.0 equiv) was added in the presence of nitrogen gas. The mixture was sealed in the tube, and heated at 100° C. for 1.5 hours. The titled compound was isolated as white solid (0.090 g, 0.20 mmol, 40% yield) after workup and purification. A side product, 6-morpholin-4-yl-2-[2-(pyridin-2-yloxy)-ethoxy]-7,9-dihydro-purine-8-thione, was also isolated as a white solid (0.018 g, 0.048 mmol, 10% yield).

### Example 64

### Preparation of (3-methoxyphenyl)-{6-Morpholin-4-yl-2-[2-(pyridin-2-yloxy)-ethoxy]-9H-purin-8-yl}-amine

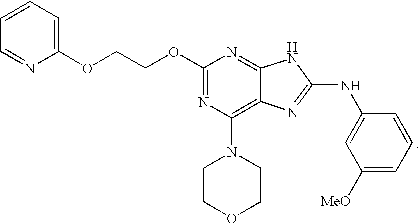

The title compound was synthesized as light brown solid in the same manner as described in Example 63, Method A.

¹H NMR (300 MHz, DMSO-d₆), δ (ppm): 11.73 (s, 1H), 9.28 (s, 1H), 8.16-8.18 (m, 1H), 7.69-7.75 (m, 1H), 7.58 (s, 1H), 7.15 (t, J=8.4 Hz, 1H), 6.97-7.01 (m, 2H), 6.85 (d, J=8.4 Hz, 1H), 6.44-6.47 (m, 1H), 4.50-4.60 (m, 4H), 4.10 (br s, 4H), 3.73 (s, 3H), 3.66-3.72 (m, 4H).

ESMS calcd for C₂₃H₂₄N₇O₄: 463.2; Found: 462.2 (M−H)⁻.

### Example 65

### Preparation of {6-Morpholin-4-yl-2-[2-(pyridin-2-yloxy)-ethoxy]-9H-purin-8-yl}-p-tolyl-amine

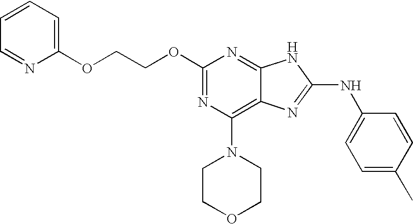

The title compound was synthesized as light brown solid in the same manner as described in Example 63, Method A.

¹H NMR (300 MHz, acetone-d₆), δ (ppm): 10.6 (s, 1H), 8.45 (br s, 1H), 8.11-8.20 (m, 1H), 7.58-7.70 (m, 3H), 7.05-7.15 (m, 2H), 6.92-6.97 (m, 1H), 6.75-6.80 (m, 1H), 4.57-4.67 (m, 4H), 4.18 (br s, 4H), 3.72-3.78 (m, 4H), 2.26 (s, 3H).

ESMS calcd for C₂₃H₂₆N₇O₃: 448.2; Found: 448.2 (M+H)⁺.

### Example 66

### Preparation of N²-[2-(3,4-Dimethoxy-phenyl)-ethyl]-6-morpholin-4-yl-N⁸-p-tolyl-9H-purine-2,8-diamine

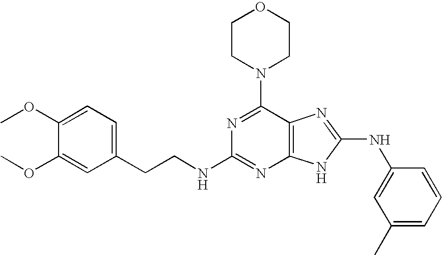

The title compound was synthesized by the method shown in Scheme 3

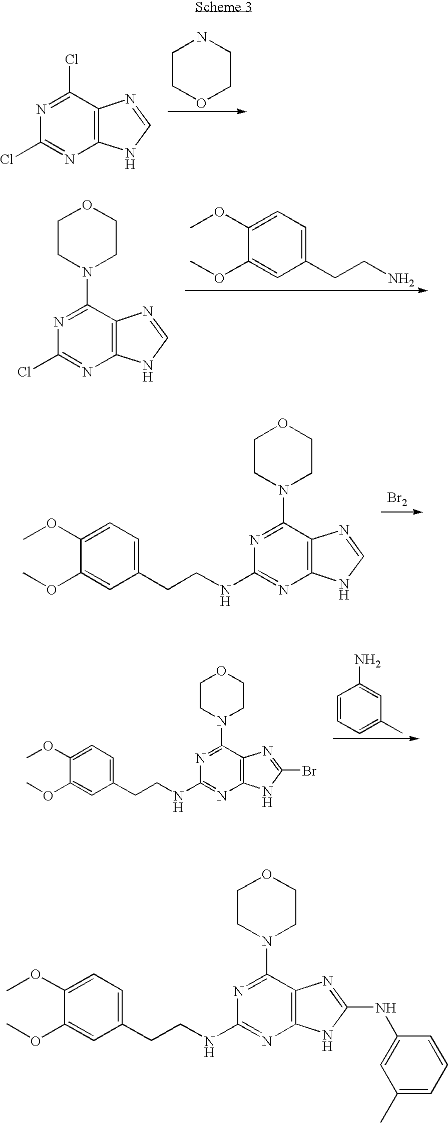

As shown in Scheme 3 above, a mixture of 2,6-dichloropurine (1.90 g, 10 mmol) and morpholine (2.34 g, 30 mmol) in water (25 mL) was heated under reflux for 15 min. Solidified reaction mixture was cooled to room temperature. Solid was filtered out and washed with water, methanol and ether. The 2-chloro-6-morpholin-4-yl-9H-purine was obtained in 96% yield (2.30 g). A mixture of 2-chloro-6-morpholin-4-yl-9H-purine (1.92 g, 8 mmol) and 2-(3,4-dimethoxyphenyl)ethylamine (4.35 g, 24 mmol) in sealed tube and under nitrogen was stirred at 190-195° C. for 1 hour. The reaction mixture turned to clear solution initially and then formed a slurry. The reaction mixture was cooled to room temperature diluted with methanol (8 mL) and the solid was collected by filtration, washed with methanol and Et₂p and dried to afford 2.30 g (740% yield) of [2-(3,4-dimethoxy-phenyl)-ethyl]-(6-morpholin-4-yl-9H-purin-2-yl)amine.

¹H NMR (DMSO-d₆) δ (ppm), 12.22 (bs, 1H), 7.69 (d, J=9.0 Hz, 1H), 6.86-6.73 (m, 3H), 6.30-6.22 (m, 1H), 4.12 (bs, 4H), 3.74-3.69 (m, 10H), 3.43 (t, J=6.0 Hz, 2H), 2.78-2.73 (m, 2H).

ESMS calcd for C₁₉H₂₄N₆O₃: 384.19; Found: 385.2 (M+H)⁺.

To a solution of [2-(3,4-dimethoxy-phenyl)-ethyl]-(6-morpholin-4-yl-9H-purin-2-yl)amine (1.16 g, 3 mmol) in dioxane (75 mL) was added bromine (0.180 mL, 3.3 mmol) in dioxane (5 mL) dropwise over a period of 1 hour. The mixture was stirred at room temperature for additional 4 hours and diluted with water (25 mL) and extracted with EtOAc. The organic phase was washed with brine, water, dried over Na₂SO₄. The solvent was evaporated in vacuo and solid was washed with methanol to give (8-bromo-6-morpholin-4-yl-9H-purin-2-yl)-[2-(3,4-dimethoxy-phenyl)-ethyl]-amine as a white solid (1.05 g, 75% yield).

¹H NMR (DMSO-d₆) δ (ppm), 6.86-6.72 (m, 3H), 6.50-6.42 (m, 1H), 4.05 (bs, 4H), 3.75-3.69 (m, 10H), 3.44-3.38 (m, 2H), 2.78-2.74 (m, 2H).

ESMS calcd for C₁₉H₂₃BrN₆O₃: 462.10; Found: 463.0 (M+H)⁺.

A mixture of (8-bromo-6-morpholin-4-yl-9H-purin-2-yl)-[2-(3,4-dimethoxy-phenyl)-ethyl]-amine (0.93 g, 2 mmol) and m-toluidine (0.86 mL, 8 mmol) in sealed tube and under nitrogen was stirred at 190-195° C. for 1 hour. The reaction mixture was cooled to room temperature diluted with methanol (5 mL) and the solid was collected by filtration, washed with small amount of methanol and Et₂O and dried to give 0.76 g of N²-[2-(3,4-Dimethoxy-phenyl)-ethyl]-6-morpholin-4-yl-N⁸-p-tolyl-9H-purine-2,8-diamine in 78% yield.

¹H NMR (DMSO-d₆) δ (ppm), 11.62 (bs, 1H), 9.46 (s, 1H), 7.38-7.18 (m, 4H), 6.86-6.70 (m, 4H), 3.82-3.34 (m, 16H), 2.77 (t, J=6.0 Hz, 2H), 2.27 (s, 3H).

ESMS calcd for C₂₆H₃₁N₇O₃: 489.25; Found: 490.2 (M+H)⁺.

### Example 67

### Preparation of 6-morpholin-4-yl-N⁸-m-tolyl-9H-purine-2,8-diamine

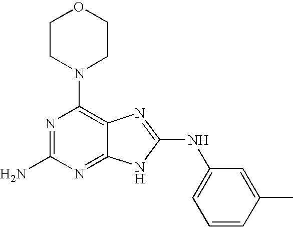

The title compound was prepared by a method as delineated herein.

¹H NMR (DMSO-d₆) δ (ppm), 9.15 (bs, 1H), 7.40-7.32 (m, 2H), 7.19-7.16 (m, 1H), 6.76-6.74 (m, 1H), 3.97 (bs, 4H), 3.74-3.72 (m, 4H), 2.27 (s, 3H).

ESMS calcd for C₁₆H₁₉N₇O: 325.17; Found: 326.1 (M+H)⁺.

### Example 68

### Preparation of 2-(6-morpholin-4-yl-8-m-tolylamino-9H-purin-2-ylamino)-ethanol

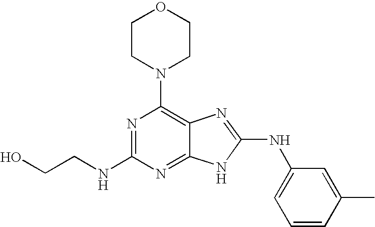

The title compound was prepared by a method as delineated herein.

¹H NMR (DMSO-d₆) δ (ppm), 11.64 (bs, 1H), 9.49 (s, 1H), 7.39-7.34 (m, 2H), 7.21 (t, J=7.2 Hz, 1H), 6.86-6.80 (m, 1H), 3.90-3.72 (m, 8H), 3.55 (f, J=6.0 Hz, 2H), 3.42-3.38 (m, 2H), 2.29 (s, 3H).

ESMS calcd for C₁₈H₂₃N₇O₂: 369.19; Found: 370.1 (M+H)⁺.

### Example 69

### Preparation of N²-[2-(3,4-Dimethoxy-phenyl)-ethyl]-6-morpholin-4-yl-N⁸-m-tolyl-9H-purine-2,8-diamine

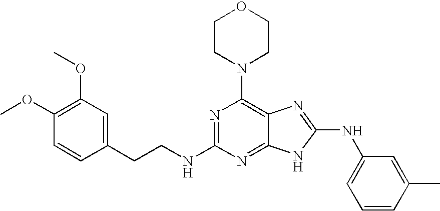

The title compound was prepared by a method as delineated herein.

¹H NMR (DMSO-d₆) δ (ppm), 11.65 (bs, 1H), 9.50 (s, 1H), 7.42-7.20 (m, 4H), 6.84-6.65 (m, 4H), 3.82-3.40 (m, 16H), 2.82-2.78 (m, 2H), 2.28 (s, 3H).

ESMS calcd for C₂₆H₃₁N₇O₃: 489.25; Found: 490.2 (M+H)⁺.

### Example 70

### Preparation of N²-[2-(3,4-dimethoxy-phenyl)-ethyl]-6-morpholin-4-yl-N-p-tolyl-9H-purine-2,8-diamine

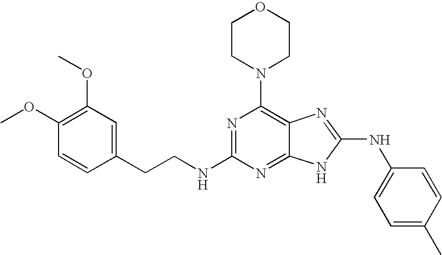

The title compound was prepared by a method as delineated herein.

¹H NMR (DMSO-d₆) δ (ppm), 11.62 (bs, 1H), 9.46 (s, 1H), 7.38-7.18 (m, 4H), 6.86-6.70 (m, 4H), 3.82-3.34 (m, 16H), 2.77 (t, J=6.0 Hz, 2H), 2.27 (s, 3H).

ESMS calcd for C₂₆H₃₁N₇O₃: 489.25; Found: 490.2 (M+H)⁺.

### Example 71

### Preparation of 9-methyl-6-morpholin-4-yl-N⁸-m-tolyl-9H-purine-2,8-diamine

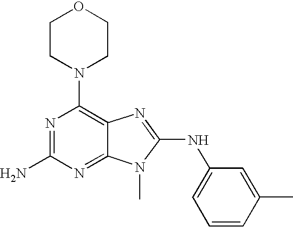

The title compound was prepared by a method as delineated herein.

¹H-NMR (DMSO-d₆) δ (ppm), 925 (bs, 1H), 7.40-7.32 (m, 2H), 7.22-7.16 (m, 2H), 6.76-6.72 (m, 1′H), 3.97 (m, 7H), 3.74-3.72 (m, 4H), 2.27 (s, 3H).

ESMS calcd for C₁₇H₂₁N₇O: 339.18; Found: 340.2 (M+H)⁺.

### Example 72

### Preparation of [2-(3,4-dimethoxy-benzyloxy)-6-morpholin-4-yl-9H-purin-8-yl]-p-tolyl-amine

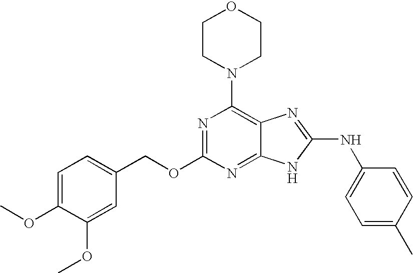

The title compound was prepared by a method as delineated herein.

¹H NMR (DMSO-d₆) δ (ppm), 11.63 (s, 1-H), 9.03 (s, 1H), 748-7.45 (m, 2H), 7.08-6.94 (m, 5H), 5.10 (s, 2H), 3.74-3.69 (m, 14H), 2.23 (s, 3H).

ESMS calcd for C₂₅H₂₈N₆O₄: 476.22; Found: 477.2 (M+H)⁺.

### Example 73

### Preparation of N²-(4-methoxy-phenyl)-N²-methyl-6-morpholin-4-yl-N⁸-m-tolyl-9H-purine-2,8-diamine

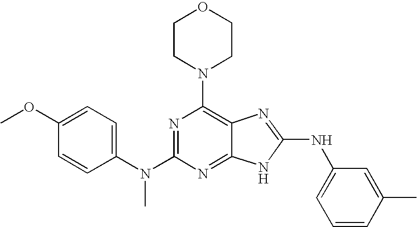

The title compound was prepared by a method as delineated herein.

¹H NMR (CDCl₃) δ (ppm), 9.37 (bs, 1H), 7.33-7.25 (m, 2H), 7.16-7.09 (m, 3H), 7.02-6.98 (m, 2H), 6.84-6.82 (m, 1H), 4.06-3.82 (m, 10H), 3.48-3.40 (m, 4H), 2.25 (s, 3H).

ESMS calcd for C₂₄H₂₇N₇O₂: 445.22; Found: 446.2 (M+H)⁺.

### Example 74

### Preparation of N²-(4-methoxy-phenyl)-N²-methyl-9-methyl-6-morpholin-4-yl-N⁸-m-tolyl-9H-purine-2,8-diamine

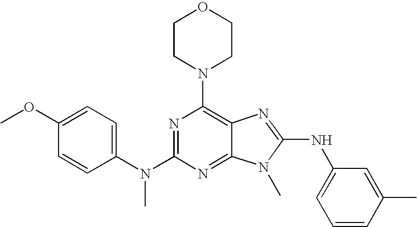

The title compound was prepared by a method as delineated herein.

¹H NMR (CDCl₃) δ (ppm), 7.38-7.07 (m, 5H), 6.95-6.8 (m, 3H), 5.94 (s, 1H), 4.20-4.05 (m, 4H), 3.81 (s, 3H), 3.78-3.75 (m, 4H), 3.51 (s, 3H), 3.44 (s, 3H), 2.30 (s, 3H).

ESMS calcd for C₂₅H₂₉N₇O₂: 459.24; Found: 460.2 (M+H)⁺.

### Example 75

### Preparation of N²-[4-(2-Methoxy-ethoxy)-phenyl]-N²-methyl-6-morpholin-4-yl-N⁸-m-tolyl-9H-purine-2,8-diamine

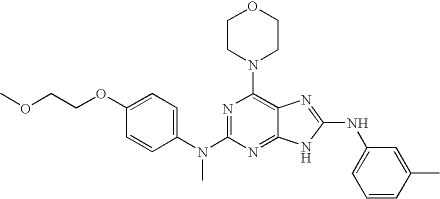

The title compound was prepared by a method as delineated herein.

¹H NMR (CDCl₃) δ (ppm), 9.20 (bs, 1H), 7.33-7.25 (m, 2H), 7.18-7.14 (m, 2H), 7.06-7.03 (m, 2H), 6.86-6.82 (m, 2H), 4.20-4.05 (m, 4H), 3.90-3.72 (m, 8H), 3.52 (s, 3H), 3.45 (s, 3H), 2.25 (s, 3H).

ESMS calcd for C₂₆H₃₁N₇O₃: 489.25; Found: 490.2 (M+H)⁺.

### Example 76

### Preparation of 4-[2-(6-morpholin-4-yl-8-m-tolylamino-9H-purin-2-ylamino)-ethyl]-benzenesulfonamide

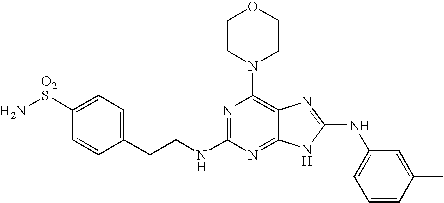

The title compound was prepared by a method as delineated herein.

¹H NMR (DMSO-d₆) δ (ppm), 11.64 (bs, 1H), 9.50 (s, 1H), 7.73 (d, J=8.1 Hz, 2H), 7.42-7.17 (m, 8H), 6.82 (bs, 1H), 3.82-3.36 (m, 10H), 2.92 (t, J=7.2 Hz, 2H), 2.27 (s, 3H).

ESMS calcd for C₂₄H₂₈N₈O₃S: 508.20; Found: 509.2 (M+H)⁺.

### Example 77

### Preparation of 2-[methyl-(6-morpholin-4-yl-8-m-tolylamino-9H-purin-2-yl)-amino]-ethanol

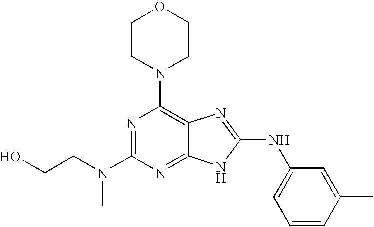

The title compound was prepared by a method as delineated herein.

¹H NMR (DMSO-d₆) δ (ppm), 9.60 (s, 1H), 7.43-7.22 (m, 3H), 6.86-6.82 (m, 1H), 6.60-6.50 (m, 1H), 4.33 (t, J=7.2 Hz, 2H), 3.94-3.72 (m, 10H), 2.99 (s, 3H), 2.29 (s, 3H).

ESMS calcd for C₁₉H₂₅N₇O₂: 383.21; Found: 384.2 (M+H)⁺.

### Example 78

### Preparation of 2-[(2-hydroxy-ethyl)-(6-morpholin-4-yl-8-m-tolylamino-9H-purin-2-yl)-amino]-ethanol

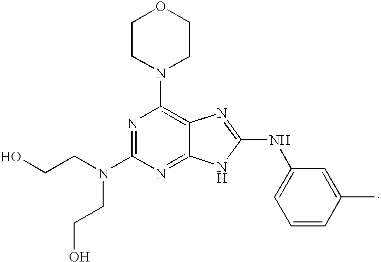

The title compound was prepared by a method as delineated herein.

¹H NMR (CDCl₃) δ (ppm), 10.62 (bs, 1H), 9.46 (s, 1H), 7.38-7.07 (m, 4H), 4.24-4.15 (m, 4H), 3.94-3.90 (m, 4H), 3.82-3.77 (m, 8H), 2.27 (s, 3H).

ESMS calcd for C₂₀H₂₇N₇O₃: 413.22; Found: 414.4 (M+H)⁺.

### Example 79

### Preparation of 6-morpholin-4-yl-N²,N⁸-di-m-tolyl-9H-purine-2,8-diamine

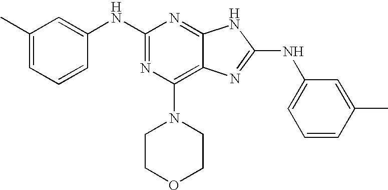

The title compound was prepared by the method shown in Scheme 4.

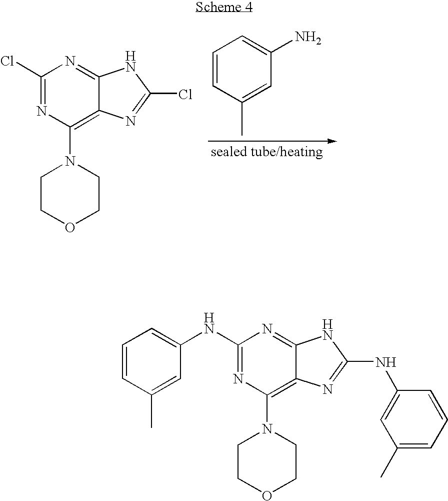

As shown in Scheme 4, a mixture of 2,8-dichloro-6-morpholin-4-yl-9H-purine (412 mg, 1.5 mmol) and m-tolylamine (0.97 mL, 9.0 mmol, 6 equiv.) was placed into a sealed tube filled with N₂. The sealed tube was submerged into an oil bath (180° C.). After 1.5 hours, the mixture in the sealed tube solidified. The sealed tube was cooled down to room temperature followed by adding ethyl acetate (10 mL) into the mixture. The resulting suspension was stirred for 1 hour at room temperature. The solid was collected by filtration and washed with cold methanol/water (5:1) and ethyl acetate. A total of 480 mg pale yellow powder was obtained. Yield was 78%.

¹H NMR (CD₃OD) δ (ppm), 7.20-7.42 (m, 6H), 6.85-7.00 (m, 2H), 3.96-3.99 (m, 4H), 3.80-3.85 (m, 4H), 2.34-2.35 (m, 6H).

ESMS calcd for C₂₃H₂₅N₇O: 415.21; Found: 416.2 (M+H)⁺.

### Example 80

### Preparation of 6-morpholin-4-yl-N²,N⁸-di-o-tolyl-9H-purine-2,8-diamine

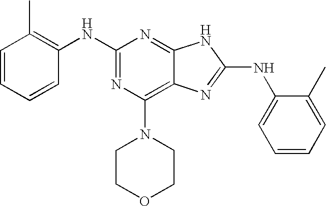

The title compound was prepared by a method as delineated herein.

¹H-NMR (CDCl₃) δ (ppm), 7.98 (br, 1H), 7.58 (br, 1H), 6.98-7.11 (m, 8H), 6.44 (br, 1H), 4.00-4.11 (m, 4H), 3.70-3.80 (m, 4H), 2.15-2.39 (m, 6H)⁺.

ESMS calcd for C₂₃H₂₅N₇O: 415.21; Found: 416.2 (M+H)⁺.

### Example 81

### Preparation of 6-morpholin-4-yl-N²,N⁸-di-p-tolyl-9H-purine-2,8-diamine

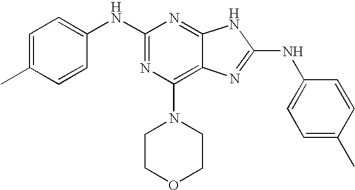

The title compound was prepared by a method as delineated herein:

¹H-NMR (CD₃OD) δ (ppm), 7.34-7.45 (dd, J=8.4, 25.8 Hz, 4H), 7.15-7.21, (dd, J=8.4, 9.0 Hz, 4H), 3.92 (m, 4H), 3.80-3.83 (m, 4H), 2.32-2.34 (m, 6H)⁺.

ESMS calcd for C₂₃H₂₅N₇O: 415.21; Found. 416.2 (M+H)⁺.

### Example 82

### Preparation of N²,N⁸-bis-(3,4-dimethoxy-phenyl)-6-morpholin-4-yl-9H-purine-2,8-diamine

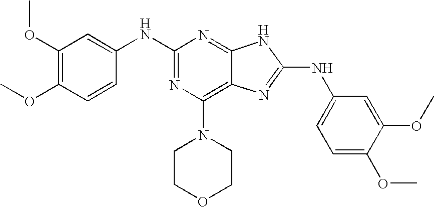

The title compound was prepared by a method as delineated herein.

¹H NMR (DMSO-d₆) δ (ppm), 7.43 (br, 1H), 7.27 (br, 1H), 6.34-7.09 (m, 7H), 3.75-4.00 (m, 20H).

ESMS calcd for C₂₅H₂₉N₇O₅: 507.22; Found: 508.2 (M+H)⁺.

### Example 83

### Preparation of N²,N⁸-bis-(3,4-dimethoxy-phenyl)-6-morpholin-4-yl-9H-purine-2,8-diamine

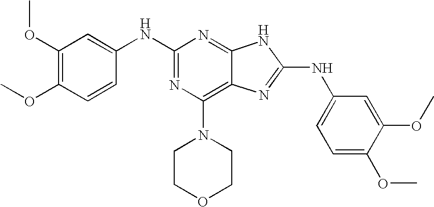

The title compound was prepared by a method as delineated herein.

¹H NMR (acetone-d₆) δ (ppm), 10.55 (br, 1H), 8.46 (d, J=8.1 Hz, 2H), 7.92 (br, 1H), 7.29 (br, 1H), 6.85 (m, 2H), 6.65 (m, 2H), 4.25 (m, 4H), 3.75-3.89 (m, 10H), 2.28 (m, 6H)/

ESMS calcd for C₂₅H₂₉N₇O₃: 475.23; Found: 476.2 (M+H)⁺.

### Example 84

### Preparation of N²,N⁸-bis-(3-methoxy-phenyl)-6-morpholin-4-yl-9H-purine-2,8-diamine

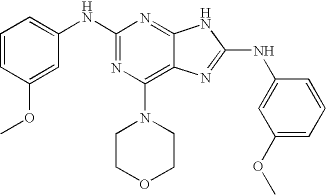

The title compound was prepared by a method as delineated herein.

¹H NMR (CD₃OD) δ (ppm), 7.19-7.35 (m, 4H), 7.02-7.05 (m, 2H), 6.64-6.74 (m, 2H), 4.00 (m, 4H), 3.80-3.85 (m, 10H).

ESMS calcd for C₂₃H₂₅N₇O₃: 447.20; Found: 448.2 (M+H)⁺.

### Example 85

### Preparation of 6-morpholin-4-yl-N2,N⁸-di-pyridin-3-yl-9H-purine-2,8-diamine

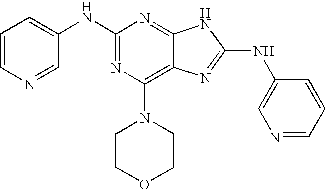

The title compound was prepared by a method as delineated herein.

¹H-NMR (CD₃OD) δ (ppm), 9-42 (s, 1H), 9.27 (d, J=5.4 Hz, 1H), 9.15 (s, 1H), 9.00 (d, J=5.4 Hz, 1H), 7.73-7.80 (m. 4H), 4.42 (m, 4H), 3.86-3.90 (m, 10H).

ESMS calcd for C₁₉H₁₉N₉O: 389.17; Found: 390.1 (M+H)⁺.

### Example 86

### Preparation of N²,N⁸-bis-(3-fluorophenyl)-6-morpholin-4-yl-9H-purine-2,8-diamine

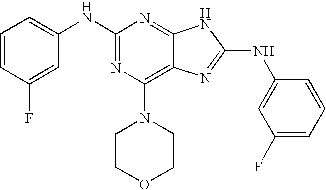

The title compound was prepared by a method as delineated herein.

¹H NMR (DMSO-d₆) δ (ppm), 9.58 (br, 1H); 9.28 (br, 1H), 7.78 (d, J=9.3 Hz, 1H), 7.59 (d, J=9.3 Hz, 1H), 7.25-7.42 (m, 4H), 6.68-6.71 (m, 2H), 4.09 (m, 4H), 3.75-3.77 (m, 4H)/

ESMS calcd for C₂₁H₁₉F₂N₇O: 423.16; Found: 424.1 (M+H)⁺.

### Example 87

### Preparation of N²,N⁸-bis-(4-methoxy-phenyl)-6-morpholin-4-yl-9H-purine-2,8-diamine

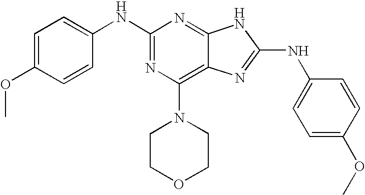

The title compound was prepared by a method as delineated herein.

¹H-NMR (DMSO-d₆) δ (ppm), 9.40 (br, 2H), 7.52 (m, 4H), 6.90 (m, 4H), 3.60-3.90 (m, 14H).

ESMS calcd for C₂₃H₂₅N₇O₃: 447.20; Found: 448.2 (M+H)⁺.

### Example 88

### Preparation of N²,N⁸-bis-(3-ethoxy-phenyl)-6-morpholin-4-yl-9H-purine-2,8-diamine

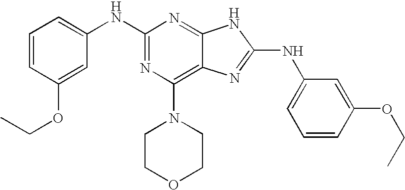

The title compound was prepared by a method as delineated herein.

¹H NMR (DMSO-d₆) δ (ppm), 9.40 (br, 2H), 7.48-7.54 (m, 2H), 6.90-7.20 (m, 4H), 6.55 (m, 2H), 3.75-4.10 (m, 12H), 1.33 (t, J=6.9 Hz, 6H).

ESMS calcd for C₂₅H₂₉N₇O₃: 475.23; Found: 476.2 (M+H)⁺.

### Example 89

### Preparation of N²,N⁸-bis-(3,5-dimethyl-phenyl)-6-morpholin-4-yl-9H-purine-2,8-diamine

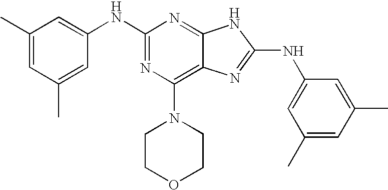

The title compound was prepared by a method as delineated herein.

¹H-NMR (CD₃OD/DMSO-d₆) δ (ppm), 7.37 (s, 4H), 7.22 (s, 4H), 6.55 (m. 2H), 6.49 (m, 2H), 4.15 (m, 4H), 3.74-3.77 (m, 4H), 2.22 (m, 12H).

ESMS calcd for C₂₅H₂₉N₇O: 443.24; Found: 444.2 (M+H)⁺.

### Example 90

### Preparation of 9-methyl-6-morpholin-4-yl-N²,N⁸-di-m-tolyl-9H-purine-2,8-diamine

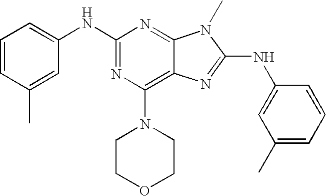

The title compound was prepared by a method as delineated herein.

¹H-NMR (CD₃OD) δ (ppm), 7.45 (m, 2H), 7.11-7.22 (m, 4H), 6.77-6.82 (m, 2H), 4.19 (m, 4H), 3.82 (m, 4H), 3.52 (s, 3H), 2.30 (m, 6H).

ESMS calcd for C₂₄H₂₇N₇O: 429.23; Found: 430.2 (M+H)⁺.

### Example 91

### Preparation of 6-Morpholin-4-yl-N²,N⁸-diphenyl-9H-purine-2,8-diamine

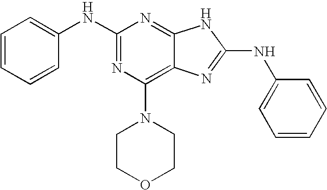

The title compound was prepared by a method as delineated herein.

¹H-NMR (DMSO-d₆) δ (ppm), 9.62 (br, 2H), 7.59 (m, 4H), 7.33 (m, 4H), 7.05 (m, 2H), 3.99 (m, 4H), 3.76 (m, 4H).

ESMS calcd for C₂₁H₂₁N₇O: 387.18; Found: 388.2 (M+H)⁺.

### Example 92

### Preparation of 6-morpholin-4-yl-N²,N⁸-bis-(3-trifluoromethyl-phenyl)-9H-purine-2,8-diamine

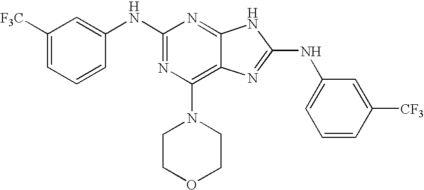

The title compound was prepared by a method as delineated herein.

¹H-NMR (DMSO-d₆) δ (ppm), 9.75 (br, 1H), 9.42 (br, 1H), 8.31 (m, 2H), 7.80 (m, 2H), 7.49 (m, 2H), 7.21 (m, 2H), 4.11 (m, 4H), 3.75 (m, 4H).

ESMS calcd for C₂₃H₁₉F₆N₇O: 523.16; Found: 524.2 (M+H)⁺.

### Example 93

### Preparation of N²,N⁸-bis-(4-chloro-phenyl)-6-morpholin-4-yl-9H-purine-2,8-diamine

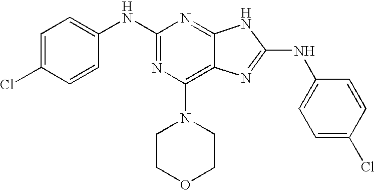

The title compound was prepared by a method as delineated herein.

¹H NMR (DMSO-d₆) δ (ppm), 9.75 (br, 2H), 7.64 (m, 4H), 7.36 (m, 4H), 4.02 (m, 4H), 3.75 (m, 4H).

ESMS calcd for C₂₁H₁₉Cl₂N₇O: 455.10; Found: 456.0 (M+H)⁺.

### Example 94

### Preparation of N²,N⁸-bis-(4-methoxy-phenyl)-N²,N⁸-dimethyl-6-morpholin-4-yl-9H-purine-2,8-diamine

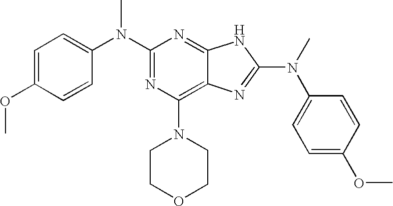

The title compound was prepared by a method as delineated herein.

¹H NMR (acetone-d₆) δ (ppm), 10.15 (br, 1H), 7.27 (AB, J=8.7 Hz, 2H), 7.21 (AB, J=8.7 Hz, 2H), 6.94 ((AB, J=8.7 Hz, 2H), 6.86 (AB, J=8.7 Hz, 2H), 4.04 (m, 4H), 3.79 (m, 6H), 3.68 (m, 4H), 3.38 (m, 6H).

ESMS clcd for C₂₅H₂₉N₇O₃: 475.23; Found: 476.5 (M+H)⁺.

### Example 95

### Preparation of 3-bromo-4-(6-morpholin-4-yl-8-m-tolylamino-9H-purin-2-ylamino)-benzenesulfonamide

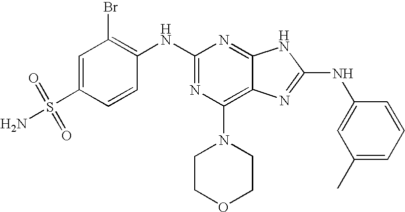

The title compound was prepared by a method as delineated herein.

¹H NMR (CD₃OD) δ (ppm), 8.68 (d, J=8.7 Hz, 1H), 8.04 (d, J=2.1 Hz, 1H), 7.76 (dd, J=2.1, 8.7 Hz, 1H), 7.49 (s, 1H), 7.34 (m, 1H), 7.16 (t, J=8.1 Hz, 1H), 6.77 (d, J=8.1 Hz), 4.18 (m, 4H), 3.83 (m, 4H), 2.30 (s, 3H).

ESMS calcd for C₂₂H₂₃BrN₈O₃S: 558.08; Found: 559.0 (M+H)⁺.

### Example 96

### Preparation of N²-(4-methanesulfonyl-phenyl)-6-morpholin-4-yl-N⁸-m-tolyl-9H-purine-2,8-diamine

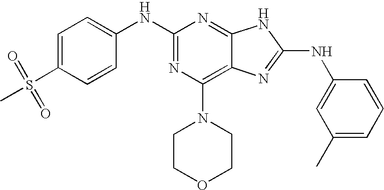

The title compound was prepared by a method as delineated herein.

¹H-NMR (DMSO-d₆) δ (ppm), 9.52 (br, 1H), 9.23 (br, 1H), 7.93 (m, 2H), 7.75 (m, 2H), 7.34-7.41 (m, 2H), 7.17 (m, 1H), 6.77 (m, 1H), 4.07 (m, 4H), 3.75 (m, 4H), 3.13 (s, 3H), 2.28 (s, 3H).

ESMS calcd for C₂₃H₂₅N₇O₃S: 479.17; Found: 480.2 (M+H)⁺.

### Example 97

### Preparation of 4-[methyl-(6-morpholin-4-yl-8-m-tolylamino-9H-purin-2-yl)-amino]-benzonitrile

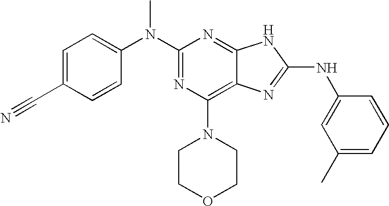

The title compound was prepared by a method as delineated herein.

¹H NMR (CD₃OD) δ (ppm), 7.37-7.59 (m, 6H), 7.21 (m, 1H), 6.81 (m, 1H), 4.15 (m, 4H), 3.83 (m, 4H), 3.59 (s, 3H), 2.35 (s, 3H).

ESMS calcd for C₂₄H₂₄N₈O: 440.21; Found: 441.2 (M+H)⁺.

### Example 98

### Preparation of N²-dimethyl-6-morpholin-4-yl-N²,N⁸-di-m-tolyl-9H-purine-2,8-diamine

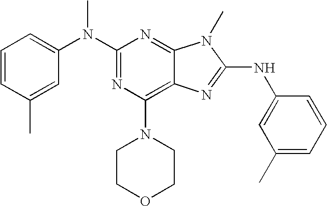

The title compound was prepared by a method as delineated herein.

¹H NMR (DMSO-d₆) δ (ppm), 9.58 (br, 2H), 7.58 (m, 1H), 7.52 (s, 1H), 7.38 (s, 1H), 7.24-7.28 (m, 3H), 6.91-6.99 (m, 2H), 3.84 (s, 3H), 3.69 (m, 4H), 3.65 (s, 3H), 3.58 (m, 4H), 2.34 (s, 3H), 2.32 (s, 3H).

ESMS calcd for C₂₅H₂₉N₇O: 443.24; Found: 444.2 (M+H)⁺.

### Example 99

### Preparation of [2-(4-Fluoro-phenoxy)-6-morpholin-4-yl-9H-purin-8-yl]-m-tolyl-amine

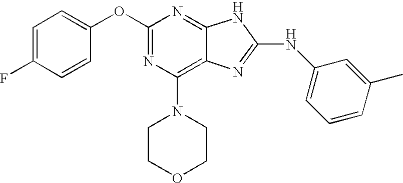

The title compound was prepared by a method as delineated herein.

¹H NMR (acetone-d₆) δ (ppm), 10.68 (s, 1H), 8.55 (s, 1H), 7.56 (s, 1H), 7.48 (d, J=8.4 Hz, 1H), 7.19-7.14 (m, 5H), 6.78 (d, J=7.2 Hz, 1H), 4.12 (m, 4H), 3.75 (m, 4H), 2.30 (s, 3H).

ESMS calcd for C₂₂H₂₁FN₆O₂: 420.17; Found: 421.1 (M+H)⁺.

### Example 100

### Preparation of (6-morpholin-4-yl-2-p-tolyloxy-9H-purin-8-yl)-m-tolyl-amine

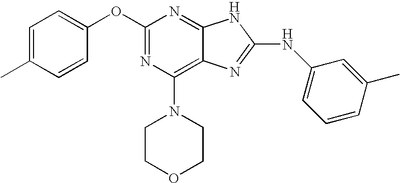

The title compound was prepared by a method as delineated herein.

¹H NMR (acetone-d₆) δ (ppm), 10.60 (s, 1H), 8.59 (s, 1H), 7.56 (s, 1H), 7.48 (d, J=9.0 Hz, 1H), 7.27-7.13 (m, 4H), 7.02 (d, J=8.4 Hz, 1H), 6.77 (d, J=8.4 Hz, 1H), 4.14 (m, 4H), 3.75 (m, 4H), 2.33 (s, 3H), 2.30 (s, 3H).

ESMS calcd for C₂₃H₂₄N₆O₂: 416.20; Found: 417.2 (M+H)⁺.

### Example 101

### Preparation of (2-chloro-6-morpholin-4-yl-9H-purin-8-yl)-m-tolyl-amine

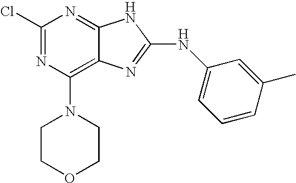

The title compound was prepared by a method as delineated herein.

¹H NMR (DMSO-d₆) δ (ppm), 12.00 (brs, 1H), 9.39 (s, 1H), 7.45 (s, 1H), 7.37 (d, J=8.1 Hz, 1H), 7.16 (t, J=7.6 Hz, 1H), 6.75 (d, J=7.2 Hz, 1H), 4.09 (m, 4H), 3.72 (m, 4H), 2.27 (s, 3H).

ESMS calcd for O₁₆H₁₇CIN₆O: 344.12; Found: 345.2 (M+H)⁺.

### Example 102

### Preparation of 3-(6-morpholin-4-yl-8-m-tolylamino-9H-purin-2-ylamino)-phenol

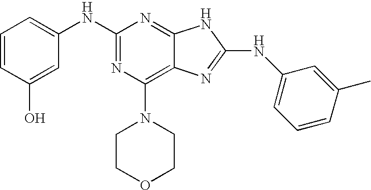

The title compound was prepared by a method as delineated herein.

¹H NMR (acetone-d₆) δ (ppm), 10.46 (brs, 1H), 8.39 (s, 1H), 8.09 (s, 1H), 7.84 (s, 1H), 7.56 (s, 1H), 7.49-7.45 (m, 2H), 7.18 (brd, J=8.7 Hz, 1H), 7.15 (t, J=7.8 Hz, 1H), 7.02 (t, J=8.0 Hz, 1H), 6.75 (brd, J=6.9 Hz, 1H), 6.37 (ddd, J=7.4, 2.1 and 0.8 Hz, 1H), 4.19 (m, 4H), 3.77 (m, 4H), 2.30 (s, 3H).

ESMS calcd for C₂₂H₂₃N₇O₂: 417.19; Found: 418.2 (M+H)⁺.

### Example 103

### Preparation of 4-(6-morpholin-4-yl-8-m-tolylamino-9H-purin-2-yloxy)-benzonitrile

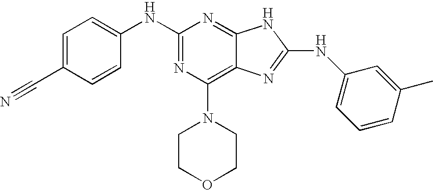

The title compound was prepared by a method as delineated herein. ¹H NMR (acetone-d₆) δ (ppm), 10.71 (brs, 1H), 8.61 (s, 1H), 7.81 (m, J_{AA}=8.7 Hz, 2H), 7.56 (S, 1H), 7.49 (brd, J=7.5 Hz, 1H), 7.36 (m, J_{AA}=8.7 Hz, 2H), 7.17 (t, J=8.0 Hz, 1H), 6.79 (d, J=7.5 Hz, 1H), 4.14 (m, 4H), 3.74 (m, 4H), 2.30 (s, 3H).

ESMS calcd for C₂₃H₂₁N₇O₂: 427.18; Found: 428.2 (M+H)⁺.

### Example 104

### Preparation of [2-(4-Methoxy-phenoxy)-6-morpholin-4-yl-9H-purin-8-yl]-m tolyl-amine

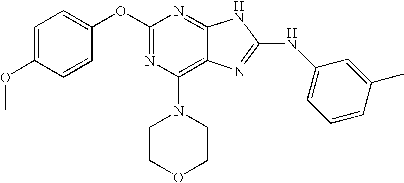

The title compound was prepared by a method as delineated herein.

¹H NMR (acetone-d₆) δ (ppm), 10.63 (brs, 1H), 8.54 (s, 1H), 7.55 (s, 1H), 7.48 (brd, J=9.0 Hz, 1H), 7.16 (t, J=7.6 Hz, 1H), 7.07 (m, J_{AA}=9 Hz, 2H), 6.93 (m, J_{BB′}=9.3 Hz, 2H), 6.76 (d, J=7.2 Hz, 1H), 4.12 (m, 4H), 3.80 (s, 3H), 3.75 (m, 4H), 2.29 (s, 3H).

ESMS calcd for C₂₃H₂₄N₆O₃: 432.19; Found: 433.2 (M+H)⁺.

### Example 105

### Preparation of N-(6-morpholin-4-yl-8-m-tolylamino-9H-purin-2-yl)-2-(pyridin-3-yloxy)-acetamide

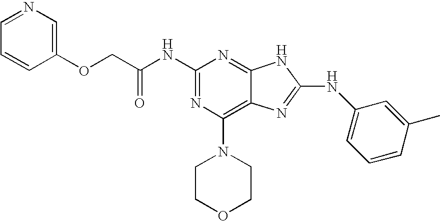

The title compound was prepared by a method as delineated herein.

¹H NMR (DMSO-d₆) δ (ppm), 10.08 (s, 1H), 9.2 (s, 1H), 8.31 (s, 1H), 8.18 (m, 1H), 7.46-7.35 (m, 4H), 7.15 (t, J=7.6 Hz, 1H), 6.73 (d, J=81 Hz, 1H), 5.10 (s, 2H), 4.11 (m, 4H). 3.73 (m, 4H), 2.27 (s, 3H).

ESMS calcd for C₂₃H₂₄N₈O₃: 460.20; Found: 461.2 (M+H)⁺.

### Example 106

### Preparation of {6-morpholin-4-yl-2-[2-(pyridin-3-yloxy)-ethoxy]-9H-purin-8-yl]-m-tolyl-amine

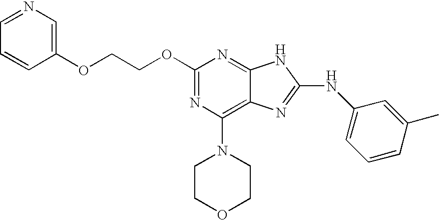

The title compound was prepared by a method as delineated herein.

¹H NMR (DMSO-d₆) δ (ppm), 11.75 (s, 1H), 9.12 (s, 1H), 8.34 (s, 1H), 8.19 (d, J=4.3 Hz, 1H), 7.46-7.35 (m, 4H), 7.17 (t, J=7.6 Hz, 1H), 6.71 (d, J=8.1 Hz, 1H), 4.54 (m, 2H), 4.38 (m, 2H), 4.08 (m, 4H), 3.71 (m, 4H), 2.27 (s, 3H).

ESMS calcd for C₂₃H₂₅N₇O₃: 447.20; Found: 448.5 (M+H)⁺.

### Example 107

### Preparation of 6-morpholin-4-yl-N²-(3-phenyl-propyl)-N⁸-m-tolyl-9H-purine-2,8-diamine

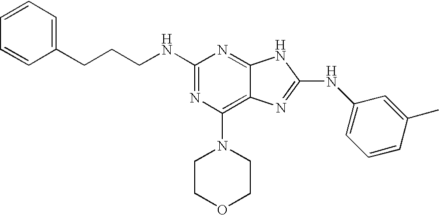

The title compound was prepared by a method as delineated herein.

¹H NMR (acetone-d₆) δ (ppm), 8.34 (brs, 1H), 7.52-7.14 (m, 9H), 6.71 (s, 1H), 5.63 (brs, 1H), 4.11 (m, 4H), 3.73 (m, 4H), 3.38 (m, 2H), 2.67 (t, J=7.8 Hz, 2H), 2.25 (s, 3H), 1.90 (qv, J=7.5 Hz, 2H).

ESMS calcd for C₂₅H₂₉N₇O: 443.24; Found: 444.2 (M+H)⁺.

### Example 108

### Preparation of N-(6-morpholin-4-yl-8-p-tolylamino-7H-purin-2-yl)-acetamide

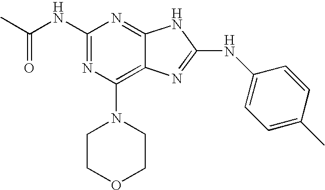

The title compound was prepared by a method as delineated herein.

¹H NMR (DMSO-d₆) δ (ppm), 11.79 (brs, 1H), 9.77 (s, 1H), 9.14 (s, 1H), 7.49 (d, J=7.8 Hz, 2H), 7.08 (d, J=7.8 Hz, 2H), 4.09 (m, 4H), 3.71 (m, 4H), 2.24 (s, 3H), 2.16 (s, 3H).

ESMS calcd for C₁₈H₂₁N₇O₂: 367.18; Found: 368.2 (M+H)⁺.

### Example 109

### Preparation of N-2′,N-8′-Bis-(3-ethyl-phenyl)-6-morpholin-4-yl-7H-purine-2,8-diamine

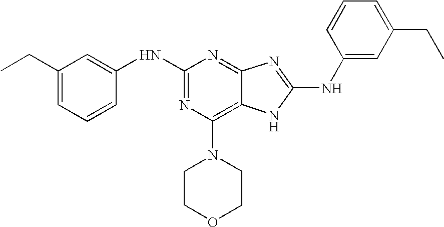

The title compound was prepared by a method as delineated herein.

¹H NMR (DMSO-d₆) δ (ppm), 9.43 (s, 1H), 7.63 (s, 1H), 7.31 (d, J=8.7 Hz, 1H), 7.18 (dd, J₁=8.7 Hz, J₂=6.9 Hz, 1H), 6.78 (d, J=6.9 Hz), 4.11 (bs, 4H), 3.72 (bs, 4H), 2.58 (q, J=7.5 Hz, 2H), 1.18 (t, J=7.5 Hz, 3H).

ESMS calcd for C₂₅H₂₉N₇O: 443.24; Found: 444.1 (M+H)⁺.

### Example 110

### Preparation of 4-methoxy-phenyl)-methyl-(6-morpholin-4-yl-8-m-tolyloxy-7H-purin-2-yl)-amine

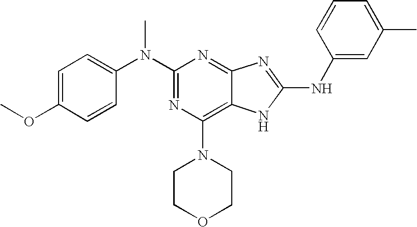

The title compound was prepared by a method as delineated herein.

¹H NMR (CDCl₃) δ (ppm), 7.26-7.21 (m, 3H), 7.07-7.04 (m, 2H), 6.97 (d, J=7.2 Hz, 1H), 4.02 (bs, 4H), 3.78 (s, 3H), 3.73 (m, 4H), 3.49 (s, 3H), 3.32 (s, 3H).

ESMS calcd for C₂₄H₂₆N₆O₃: 446.21; Found: 447.1 (M+H)⁺.

### Example 111

### Preparation of (2,6-di-morpholin-4-yl-7H-purin-8-yl)-m-tolyl-methanone

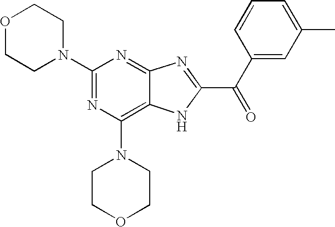

The title compound was synthesized by the method shown in Scheme 5.

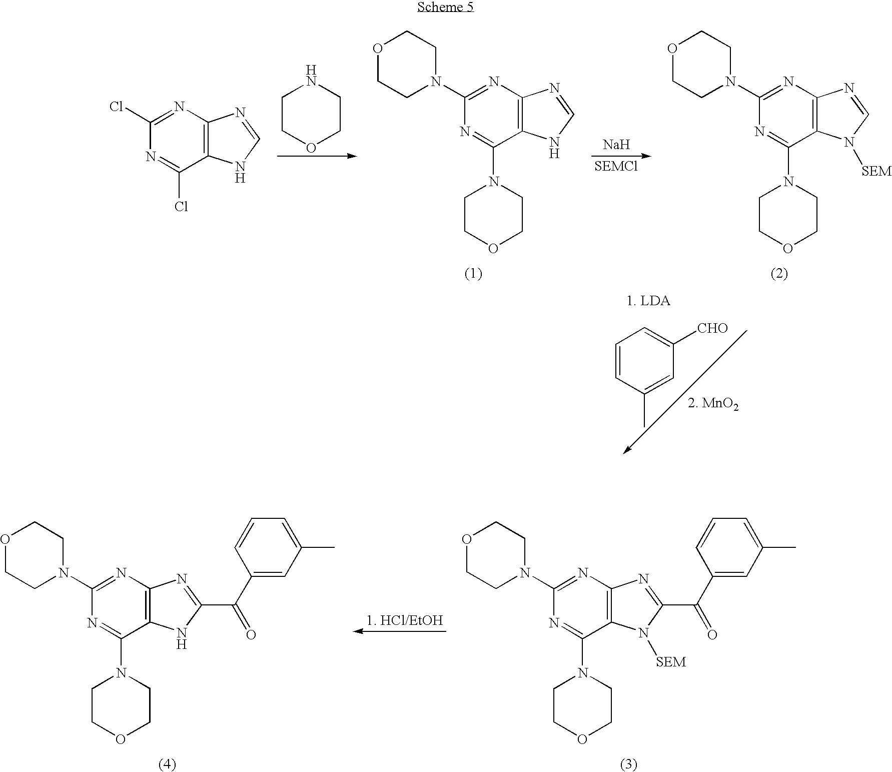

As shown in Scheme 5 above, 2,6-dichloropyrimidine (19 g, 5.29 mmol) was dissolved in morpholine (5 mL) in a sealed tube. The tube was heated to 120 C for hours then cooled to room temperature. Water (100 mL) was added and the resulting precipitate was filtered and washed with water to give 2,6-di-morpholin-4-yl-7H-purine (1.33 g, 87%). 2,6-Di-morpholin-4-yl-7H-purine (1.33 g, 4.58 mmol) was dissolved in DMF (50 mL). NaH (0.22 g, 5.50 mmol, 60% dispersion in oil) was added and the reaction was stirred at room temperature for min. 2-(Trimethylsilyl)ethoxymethyl chloride (0.92 g, 5.50 mmol) was added dropwisely and the reaction was stirred for 18 h at room temperature. Water (200 mL) then ethyl aceate (200 mL) were added. The ethyl acetate extracts were washed with water (3×100 mL), dried over MgSO₄, filtered and evaporated to dryness. The resulting residue was purified by silicagel column chromatography eluting with a gradient of 1:1 ethyl aceate to ethyl acetate to produce 2,6-Di-morpholin-4-yl-7-(2-trimethylsilanylethoxymethyl)-7H-purine (1.51 g, 78% yield).

¹H NMR (DMSO-d₆) δ (ppm), 8.23 (s, 1H), 8.18 (d, J=7.1 Hz, 1H), 7.22-7.18 (m, 4H), 6.97 (d, J=9.3 Hz, 2H), 5.78 (s, 1H), 4.15 (bs, 4H), 3.80-3.78 (m, 7H), 3.43 (s, 3H), 2.33 (s, 3H).

ESMS calcd for C₁₉H₃₂N₆O₃Si: 420.23; Found: 421.2 (M+H)⁺.

2,6-Di-morpholin-4-yl-7-(2-trimethylsilanylethoxymethyl)-7H-purine (266 mg, 0.63 mmol) was dissolved in dry THF (10 mL) and cooled to −78 C. A solution of LDA (0.38 mL, 0.76 mmol, 2 M solution in heptane) was added dropwisely then the reaction was stirred at −78 C for 30 min. To the resulting suspension was added a solution of m-tolylaldehyde (114 mg, 0.95 mmol) in THF (5 mL) then the reaction was stirred for 1 hour. Saturated NH₄Cl (50 mL) was added then the reaction was allowed to warm to room temperature. THF was removed under reduced pressure then ethyl acetate (50 mL) was added. The ethyl acetate layer was washed with water (3×50 mL), dried over MgSO₄ then evaporated to dryness. The crude product was purified by silcagel column chromatography. Elution with 25% ethyl aceatelhexane produced [2,6-di-morpholin-4-yl-7-(2-trimethylsilanyl-ethoxymethyl)-7H-purin-8-yl]-m-tolyl-methanone (198 mg, 56% yield).

¹H NMR (CDCl₃) δ (ppm), 8.11 (s, 1H), 8.07 (d, J=7.2 Hz, 1H), 7.40-7.39 (m, 2H), 5.95 (s, 2H), 3.84-3.78 (m, 16H), 3.68-3.63 (m, 2H), 2.43 (s, 3H), 0.97-0.91 (m, 2H), −0.08 (s, 9H).

ESMS calcd for C₂₇H₃₉N₆O₄Si: 538.27; Found: 539.2 (M+H)⁺.

[2,6-Di-morpholin-4-yl-7-(2-trimethylsilanyl-ethoxymethyl)-7H-purin-8-yl]-m-tolyl-m ethanone (185 mg, 0.34 mmol) was dissolved in ethanol (10 ml) and 2N HCl (4 mL). The resulting suspension was heated to reflux for 4 hrs then cooled to room temperature. After neutralization with 2N NaOH, ethanol was removed under reduced pressure and ethyl acetate (100 mL) was added. The ethyl acetate layer was washed with water (3×50 mL), dried over MgSO₄ then evaporated to dryness. The crude product was purified by silcagel column chromatography. Elution with, a gradient of 25% ethyl aceate/hexane to ethyl acetate to 10% methanol/ethyl aceate produced (2,6-di-morpholin-4-yl-7H-purin-8-yl)-m-tolyl-methanone (80 mg, 57% yield).

¹H NMR (DMSO-d₆) δ (ppm), 8.34 (s, 1H), 8.28 (d, J=7.5 Hz, 1H), 7.62-7.58 (m, 2H), 3.89-3.80 (m, 16H), 2.54 (s, 3H).

ESMS calcd for C₂₁H₂₄N₆O₃: 408.19; Found: 409.1 (M+H)⁺.

### Example 112

### Preparation of {2-[(4-Methoxy-phenylmethyl-amino]-6-morpholin-4-yl-7H-purin-8-yl}-m-tolyl-methanone

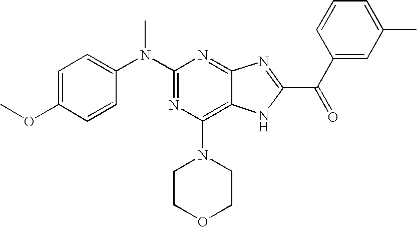

The title compound was prepared by a method as delineated herein.

¹H NMR (DMSO-d₆) δ (ppm), 8.19 (s, 1H), 8.12 (d, J=7.5 Hz, 1H), 7.46-7.43 (m, 2H), 7.25 (d, J=9.3 Hz, 2H), 6.93 (d, J=9.3 Hz, 2H), 4.04 (bs, 4H), 3.77 (s, 3H), 3.70 (bs, 4H), 3.43 (s, 3H), 2.39 (s, 3H).

ESMS calcd for C₂₅H₂₆N₆O₃: 458.21; Found: 459.1 (M+H)⁺.

### Example 113

### Preparation of (4-fluoro-5,7-di-morpholin-4-yl-1H-s-yl)-m-tolyl-amine

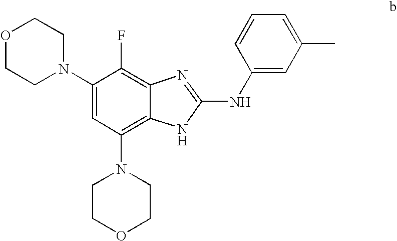

The title compound was prepared by a method as delineated herein.

¹H NMR (DMSO-d₆) δ (ppm), 7.5-7.1 (m, 5H), 3.89-3.80 (m, 16H), 2.54 (s, 3H),

ESMS calcd for C₂₂H₂₆FN₅O₂: 411.2; Found: 412.1 (M+H)⁺.

### Example 114

### Preparation of [2-(2-methoxy-ethyl)-6-morpholin-4-yl-9H-purin-8-yl]-m-tolyl-amine

The title compound was prepared by the method shown in Scheme 6.

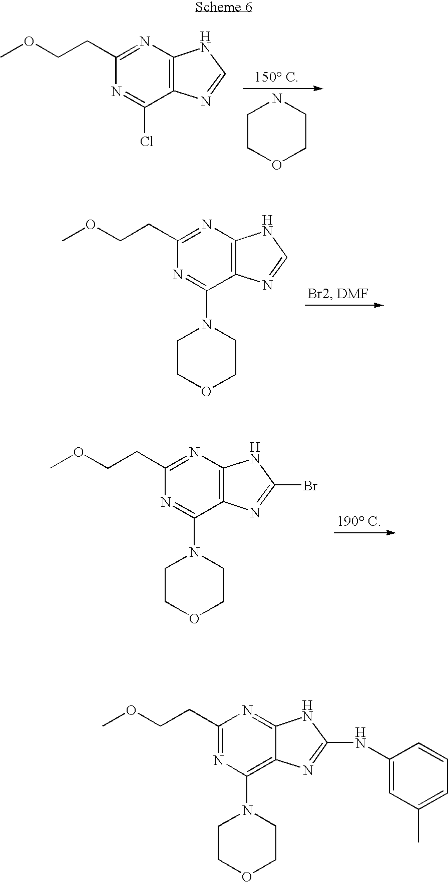

6-Chloro-2-(2-methoxy-ethyl)-9H-purine (0.5 g, 2.4 mmol, synthesized by following the procedure reported by Crespo and al. (Journal of Medicinal Chemistry, 1998, Vol. 41, No. 21, p. 4024) was heated in morpholine (1 mL, 5 eq) at 150° C. for 15 minutes. Reaction mixture was cooled to room temperature and distributed between dichloromethane and water. Organic layer was washed 2 times with water, then with brine, dried over MgSO₄ and 2-(2-methoxy-ethyl)-6-morpholin-4-yl-9H-purine (0.46 g, 75%) was isolated by column chromatography.

ESMS calcd for C₁₂H₁₇N₅O₂: 263.14; Found: 286.2 (M+23)⁺.

To a solution of 2-(2-methoxy-ethyl)-6-morpholin-4-yl-9H-purine (0.46 g, 1.7 mmol) in 1 mL of DMF bromine (0.34 g, 1.2 eq) was added dropwise, and a resulted solution was heated at 110° C. for 30 minutes. Solvent was removed in vacuo, a residue was dissolved in dichloromethane, washed with water, brine and dried over MgSO₄. Residue was purified by passing through silica gel (eluent dichloromethane:acetone:methanol 3:1:0.25) to afford 8-bromo-2-(2-methoxyethyl)-6-morpholin-4-yl-9H-purine (0.42 g, 70%).

ESMS calcd for C₁₂H₁₆BrN₅O₂: 341.05; Found: 342.0 (M+1)⁺.

A suspension of 8-bromo-2-(2-methoxy-ethyl)-6-morpholin-4-yl-9H-purine (0.42 g, 1.2 mmol) in m-toluidine (0.5 mL, 3.8 eq) in a tightly stoppered flask was heated at 190° C. for 15 minutes. Column chromatography afforded [2-(2-methoxy-ethyl)-6-morpholin-4-yl-9H-purin-8-yl]-m-tolyl-amine (0.36 g, 81%) as an off-white solid.

¹H NMR (DNSO-d₆): δ 11.70 (s, 1H), 9.24 (s, 1H), 7.47 (s, 1H), 7.38 (d, J=8.4 Hz, 1H), 7.15 (t, J=7.6 Hz, 1H), 6.73 (d, J=7.5 Hz, 1H), 4.11 (m, 4H), 3.75 (t, J=6.9 Hz, 2H), 3.73 (m, 4H), 3.24 (s, 3H), 2.88 (t, J=6.9 Hz, 2H), 2.27 (s, 3H).

ESMS calcd for C₁₉H₂₄N₆O₂: 368.20; Found: 369.1 (M+1)⁺.

### Example 115

### Preparation of N²,N⁹-bis-(3-methylphenyl)-6-(4-methylpiperidinyl)-9H-purine-2,8-diamine

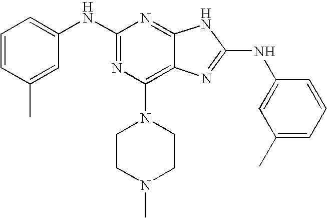

The title compound was prepared by a method as delineated herein.

¹H NMR (CD₃OD) δ (ppm), ¹H-NMR (CD₃OD) δ (ppm), 7.4-7.1 (m, 6H), 6.77-6.82 (m, 2H), 4-3.5 (m, 11H), 2.30 (m, 6H).

ESMS calcd for C₂₄H₂₈N₈: 428.24; Found: 429.2 (M+H)⁺.

### Example 116

### Preparation of 9-Methyl-6-morpholin-4-yl-N²,N⁸-di-m-tolyl-9H-purine-2,8-diamine

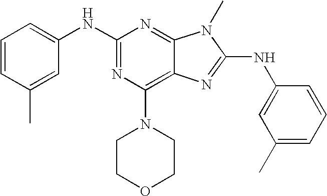

The title compound was prepared by a method as delineated herein (see, for instance, Example 79).

¹H-NMR (CD₃OD) δ (ppm), 7.45 (m, 2H), 7.11-7.22 (m, 4H), 6.77-6.82 (m, 2H), 4.19 (m, 4H), 3.82 (m, 4H), 3.52 (s, 3H), 2.30 (m, 6H); ESMS clcd for C₂₄H₂₇N₇O: 429.23; Found: 430.2 (M+H)⁺.

### Example 117

### Preparation of N²-(4-Methoxy-phenyl)-N²-methyl-6-morpholin-4-yl-N⁸-m-tolyl-9H-purine-2,8-diamine

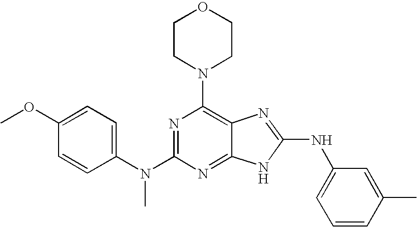

The title compound was prepared by a method as delineated herein (see, for instance, Example 79).

¹H-NMR (CDCl₃) δ (ppm), 9.37 (bs, 1H), 7.33-7.25 (m, 2H), 7.16-7.09 (m, 3H), 7.02-6.98 (m, 2H), 6.84-6.82 (m, 1H), 4.06-3.82 (m, 10H), 3.48-3.40 (m, 4H), 2.25 (s, 3H); ESMS clcd for C₂₄H₂₇N₇O₂: 445.22; Found: 446.2 (M+H)⁺.

Inhibitory Activity of Exemplary Compounds on Osteoclast Formation

### Example 118

### Materials and Methods

Human peripheral blood mononuclear cells (PBMC) were Isolated from healthy donor blood. The cells were seeded in multi-well plates at 7.5×10⁵ cells/ml in RPMI 1640 medium including 10% FBS. Osteoclast formation was induced with 20 ng/ml of recombinant human receptor activator of NF-kB-ligand (RANKL) and 10 ng/ml of human M-CSF in the presence of various doses of test compounds. After 48 hours of culture, RANKL and M-CSF was replenished and further cultured for 2 days. Then, the cultured cells were stained for tartrate-resistant acid phosphatase (TRAP). Osteoclasts were identified as TRAP-positive cells with more than 3 nuclei. Total cell viability was assessed by CCK-8 assay (Dojindo, Gaithersburg, Md.) with 24 hour incubation.

Results:

The tested compounds of this invention significantly reduced osteoclast formation as compared to two positive controls (Tamoxifen and 17β-estradiol). The obtained IC50 values (compound concentration required for 50% inhibition of osteoclast formation) and CC50 values (compound concentration required for 50% inhibition of cell viability) are shown in Table 3.

TABLE 3
IC50 and EC50 values for Osteoclast Formation and Cell Viability

| Compound from | IC50 | CC50 |
|---|---|---|
| Example No. | (nM) | (nM) |
| 63 | 3 | >1000 |
| 79 | 9 | >1000 |
| 116 | 2 | >1000 |
| 117 | 12 | >1000 |
| Tamoxifen | 474 | |
| 17β-estradiol | 78 | |

All publications, patent applications, patents, and other documents cited herein are incorporated by reference in their entirety. In case of conflict, the present specification, including definitions, will control. In addition, the materials, methods, and examples are illustrative only and not intended to be limiting.

We claim:

1. A compound selected from the group consisting of: 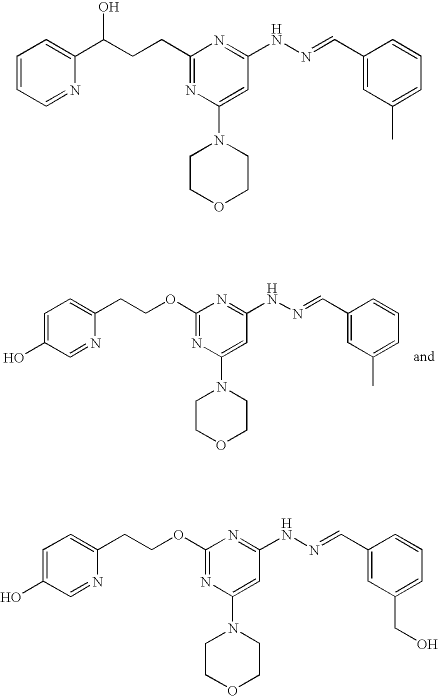
or a pharmaceutically acceptable salt, solvate, clathrate, or prodrug thereof.

2. A composition comprising an effective amount of a compound of formula (I): 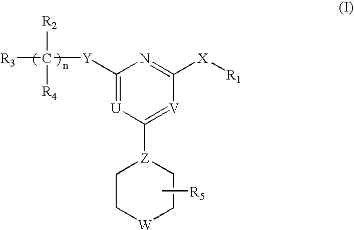
wherein
R₁ is 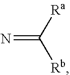
aryl, or heteroaryl;
each of R₂ and R₄, independently, is R^{c}, halogen, nitro, cyano, isothionitro, SR^{c}, or OR^{c}; or R₂ and R₄, taken together, is carbonyl.
R₃ is R^{c}, alkenyl, alkynyl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d};
R₅ is H or alkyl;
n is 0, 1, 2, 3, 4, 5, or 6;
X is O, S, S(O), S(O₂), or NR^{c};
Y is a covalent bond, CH₂, C(O), C═N—R^{c}, C═N—OR^{c}, C═N—SR^{c}, O, S, S(O), S(O₂), or NR^{c};
Z is N or CH;
one of U and V is N, and the other is CR^{c}; and
W is O, S, S(O), S(O₂), NR^{c}, or NC(O)R^{c};
in which each of R^{a} and R^{b}, independently, is H, alkyl, aryl, heteroaryl; and each of R^{c} and R^{d}, independently, is H, alkyl, aryl, heteroaryl, cyclyl, heterocyclyl, or alkylcarbonyl
or a pharmaceutically acceptable salt, solvate, clathrate, or prodrug thereof.

3. The composition of claim 2, wherein R₁ is

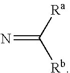

4. The composition of claim 3, wherein U is N and V is CH.

5. The composition of claim 3, wherein Z is N and W is O.

6. The composition of claim 3, wherein X is NR^{c}.

7. The composition of claim 3, wherein Y is O, S, or CH₂, and n is 0, 1, 2, 3, or 4.

8. The composition of claim 7, wherein R₃ is aryl or heteroaryl.

9. The composition of claim 7, wherein R₃ is OR^{c}, SR^{c}, C(O)OR^{c}, or C(O)NR^{c}R^{d}.

10. The composition of claim 7, wherein R₃ is 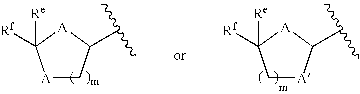
wherein
each of A and A′, independently, is O, S, or NH;
each of R^{e} and R^{f}, independently is H, alkyl, aryl, or heteroaryl; and
m is 1 or 2.

11. The composition of claim 3, wherein one of R^{a} and R^{b} is 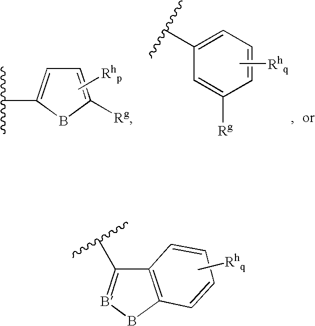
in which
B is NRⁱ, O, or S;
B′ is N or CRⁱ;
R^{g} is H, halogen, CN, alkyl, cyclyl, alkyloxy, alkylcarbonyl, alkyloxycarbonyl, aryloxycarbonyl, heteroaryloxycarbonyl, hydroxyalkyl, alkylamino, or alkylaminocarbonyl;
R^{h} is H, halogen, NO₂, CN, alkyl, aryl, heteroaryl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d};
Rⁱ is H, alkyl, or alkylcarbonyl;
p is 0, 1, or 2; and
q is 0, 1, 2, 3, or 4.

12. The composition of claim 11, wherein one of R^{a} and R^{b} is 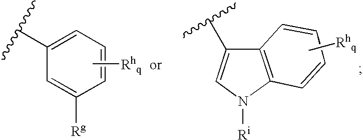
in which R⁹, R^{h}, Rⁱ, and q are as defined in claim 11; and
the other of R^{a} and R^{b} is H or alkyl.

13. The composition of claim 12, wherein
R^{g} is H, methyl, ethyl, propyl, cyclopropyl, methoxy, ethoxy, methoxycarbonyl, methylaminocarbonyl or halogen;
R^{h} is F, Cl, CN, methyl, methoxy, ethoxy, OC(O)CH₃, OC(O)C₂H₅, C(O)OH, C(O)OC₂H₅, C(O)NH₂, NHC(O)CH₃, or S(O₂)NH₂;
Rⁱ is H, methyl, ethyl, or acetyl, and
q is 0, 1, or 2.

14. The composition of claim 13, wherein U is N, V is CH, Z is N, and W is O.

15. The composition of claim 14, wherein X is NR^{c}; and R^{c} is H, methyl, ethyl, or acetyl.

16. The composition of claim 15, wherein Y is O, S, or CH₂; and n is 0, 1, 2, 3, or 4.

17. The composition of claim 16, wherein R₃ is R^{c}, OR^{c}, SR^{c}, C(O)OR^{c}, or C(O)NR^{c}R^{d}.

18. The composition of claim 17, wherein R₃ aryl, heteroaryl, hydroxyl, alkyloxy, or heteroaryloxy.

19. The composition of claim 2, wherein R₁ is aryl or heteroaryl.

20. The composition of claim 19, wherein R₁ is 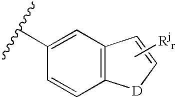
wherein
D is O, S, or NR^{m};
R^{j} is benzo, halogen, CN, hydroxyl, alkyl, aryl, heteroaryl, alkoxyl, aryloxyl, or heteroaryloxyl;
R^{m} is H, alkyl, or alkylcarbonyl; and
r is 0, 1, or 2.

21. The composition of claim 20, wherein X is NR^{c}; and R^{c} is H, methyl, ethyl, or acetyl.

22. The composition of claim 21, wherein U is N, V is CH, Z is N, and W is O.

23. The composition of claim 22, wherein Y is O, S, or CH₂; and n is 0, 1, 2, 3, or 4.

24. The composition of claim 23, wherein R₃ is aryl or heteroaryl.

25. The composition of claim 23, wherein R₃ is OR^{c}, SR^{c}, C(O)OR^{c}, or C(O)NR^{c}R^{d}.

26. The composition of claim 23, wherein R₃ is 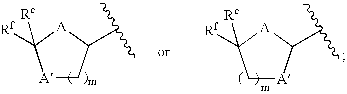
wherein
each of A and A′, independently, is O, S, or NH;
each of R^{e} and R^{f}, independently is H, alkyl, aryl, or heteroaryl; and
m is 1 or 2.

27. The compound of claim 23, wherein R₁ is 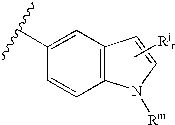
wherein
R^{m} is H, alkyl, or alkylcarbonyl;
R^{j} is methyl, ethyl, propyl, or benzo; and
r is 1 or 2.

28. The composition of claim 2, wherein
R₁ is 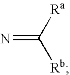
each of R₂ and R₄ is H;
R₃ is H, alkyl, aryl, heteroaryl, cyclyl, heterocyclyl, alkyloxycarbonyl, alkylaminocarbonyl, or alkylcarbonyl; and
X is NR^{c}.

29. The composition of claim 28, wherein X is NH.

30. The composition of claim 28, wherein one of R^{e} and R^{b} is H or alkyl; and the other is aryl or heteroaryl optionally substituted with R^{g} and R^{h}_{q}; R^{g} being halogen, CN, alkyl, alkyloxy, alkylcarbonyl, alkyloxycarbonyl, aryloxycarbonyl, heteroaryloxycarbonyl, hydroxyalkyl, alkylamino, or alkylaminocarbonyl; R^{h} being halogen, CN, hydroxyl, alkyl, aryl, heteroaryl, alkoxyl, aryloxyl, or heteroaryloxyl; and q being 0, 1, 2, 3, or 4.

31. The composition of claim 29, wherein one of R^{a} and R^{b} is H or alkyl; and the other is 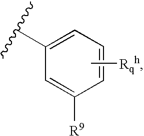
wherein
R^{g} is H, alkyl, alkoxyl, methoxycarbonyl, methylaminocarbonyl, or halogen;
R^{h} is halogen, CN, hydroxyl, alkyl, aryl, heteroaryl, alkoxyl, aryloxyl, or heteroaryloxyl; and
q is 0, 1, 2, 3, or 4.

32. The composition of claim 28, wherein U is N, V is CH, Z is N, and W is O.

33. The composition of claim 32, wherein R₃ is heteroaryl or heterocyclyl.

34. The composition of claim 33, wherein R₃ is pyridinyl.

35. The composition of claim 33, wherein R₃ is 1-oxy-pyridinyl.

36. The composition of claim 33, wherein R₃ is 1H-pyridin-2-one.

37. The composition of claim 33, wherein n is 2, and Y is O.

38. The composition of claim 37, wherein X is NH.

39. The composition of claim 38, wherein one of R^{a} and R^{b} is H or alkyl;
and the other is 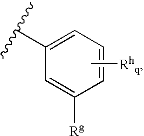
wherein
R^{g} is H, alkyl, alkoxyl, methoxycarbonyl, methylaminocarbonyl, or halogen;
R^{h} is halogen, CN, hydroxyl, alkyl, aryl, heteroaryl, alkoxyl, aryloxyl, or heteroaryloxyl; and
q is 0, 1, 2, 3, or 4.

40. The composition of claim 38, wherein one of R^{a} and R^{b} is H; and the other is 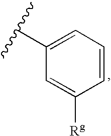
in which R^{g} is as defined in claim 39.

41. The composition of claim 2, wherein the compound is:
N-{2-[3-(3,4-dimethoxy-phenyl)-propyl]-6-morpholin-4-yl-pyrimidin-4-yl}-N′-(1H-indol-3-ylmethylene)-hydrazine,
N-(2-n-butoxy-6-morpholin-4-yl-pyrimidin-4-yl)-N′-(1H-indol-3-ylmethylene)-hydrazine,
N-(2-(4-hydroxybutyl)-6-morpholin-4-yl-pyrimidin-4-yl)-N′-(1H-indol-3-ylmethylene)-hydrazine,
N-[2-(2-[1,3]dioxan-2-yl-ethyl)-6-morpholin-4-yl-pyrimidin-4-yl]-N′-(1H-indol-3-yl methylene)-hydrazine
N-(1H-indol-3-ylmethylene)-N′-[2-(3-methoxy-propyl)-6-morpholin-4-yl-pyrimidin-4-yl]-hydrazine,
3-{4-[N′-(1H-indol-3-ylmethylene)-hydrazino]-6-morpholin-4-yl-pyrimidin-2-ylsulfanyl}-propan-1-ol,
3-{2-[N′-(1H-indol-3-ylmethylene)-hydrazino]-6-morpholin-4-yl-pyrimidin-4-ylsulfanyl}propan-1-ol,
N-[2-(2,2-dimethyl-[1,3]dioxolan-4-ylmethoxy)-6-morpholin-4-yl-pyrimidin-4-yl]-N′-(1H-indol-3-ylmethylene)-hydrazine,
N-{2-[2-(3,4-dimethoxy-phenyl)-ethoxy]-6-morpholin-4-yl-pyrimidin-4-yl}-N′-(1H-indol-3-ylmethylene)-hydrazine,
N-(1H-indol-3-ylmethylene)-N′-[6-morpholin-4-yl-2-(2-pyridin-2-yl-ethoxy)-pyrimidin-4-yl]-hydrazine,
N-(1H-indol-3-ylmethylene)-N′-[6-morpholin-4-yl-2-(3-pyridin-2-yl-propyl)-pyrimidin-4-yl]-hydrazine,
N-(3-methyl-benzylidene)-N′-[6-morpholin-4-yl-2-(2-pyridin-2-yl-ethoxy)-pyrimidin-4-yl]-hydrazine,
N-(3-ethyl-benzylidene)-N′-[6-morpholin-4-yl-2-(2-pyridin-2-yl-ethoxy)-pyrimidin-4-yl]-hydrazine,
N-(3-methyl-benzylidene)-N′-[6-morpholin-4-yl-2-(3-pyridin-2-yl-propyl)-pyrimidin-4-yl]-hydrazine,
N-[6-morpholin-4-yl-2-(2-pyridin-2-yl-ethoxy)-pyrimidin-4-yl]-N′-(1-m-tolyl-ethylidene)hydrazine,
N-[1-(1H-indol-3-yl)ethylidene]-N′-[6-morpholin-4-yl-2-(2-pyridin-2-yl-ethoxy-pyrimidin-4-yl]-hydrazine,
3-methyl-benzaldehyde O-[6-morpholin-4-yl-2-(2-pyridin-2-yl-ethoxy)-pyrimidin-4-yl]-oxime,
1H-indole-3-carbaldehyde O-[6-morpholin-4-yl-2-(2-pyridin-2-ylethoxy)-pyrimidin-4-yl]-oxime,
N-(1H-indol-3-ylmethylene)-N′-{6-morpholin-4-yl-2-[2-(pyridin-3-yloxy)-ethoxy]-pyrimidin-4-yl}-hydrazine,
N-(3-methyl-benzylidene)-N′-{6-morpholin-4-yl-2-[2-(pyridin-3-yloxy)-ethoxy]-pyrimidin-4-yl}-hydrazine,
butyl-{4-[N′-(1H-indol-3-ylmethylene)-hydrazino]-6-morpholin-4-yl-pyrimidin-2-yl}-amine,
N-(3-methyl-benzylidene)-N′-[6-morpholin-4-yl-2-(pyridin-3-yloxy)-pyrimidin-4-yl]-hydrazine,
N-(3-methylbenzlidene)-N′-(5-methyl-6-morpholin-4-yl-2-phenylpyrimidin-4-yl)hydrazine,
N-(3-methyl-benzylidene)-N′-(2-phenyl-6-thiomorpholin-4-yl-pyrimidin-4-yl)-hydrazine,
(2,3-dimethyl-1H-indole-5-yl)-{6-morpholin-4-yl-2-[2-(pyridin-3-yloxy)-ethoxy]-pyrimidin-4-yl}amine,
(2,3-dimethyl-1H-indole-5-yl)-{6-morpholin-4-yl-6-[2-(pyridin-3-yloxy)ethoxy]-pyrimidin-2-yl}amine,
3-{4-[N′-(3-methyl-benzylidene)-hydrazino]-6-morpholin-4-yl-pyrimidin-2-yl}-propionic acid ethyl ester,
N-(3-methyl-benzylidene)-N′-{6-morpholin-4-yl-2-[2-(1-oxy-pyridin-2-yl)-ethoxy]-pyrimidin-4-yl}hydrazine,
1-(2-{4-[N′-(3-methyl-benzylidene)-hydrazino]-6-morpholin-4-yl-pyrimidin-2-yloxy}-ethyl)-1H-pyridin-2-one,
N-(3-iodo-benzylidene)-N′-[6-morpholin-4-yl-2-(2-pyridin-2-yl-ethoxy)-pyrimidin-4-yl]-hydrazine,
N-(3-fluoro-benzylidene)-N′-[6-morpholin-4-yl-2-(2-pyridin-2-yl-ethoxy)pyrimidin-4-yl]-hydrazine,
N-(3-chloro-benzylidene)-N′-[6-morpholin-4-yl-2-(2-pyridin-2-yl-ethoxy)-pyrimidin-4-yl]-hydrazine,
N-(3-bromo-benzylidene)-N′-[6-morpholin-4-yl-2-(2-pyridin-2-yl-ethoxy pyrimidin-4-yl]-hydrazine,
3-{[6-morpholin-4-yl-2-(2-pyridin-2-yl-ethoxy)-pyrimidin-4-yl]hydrazonomethyl}-benzoic acid methyl ester,
1-(2-{4-[N′-(3-iodo-benzylidene)-hydrazino]-6-morpholin-4-yl-pyrimidin-2-yloxy}-ethyl)-1H-pyridin-2-one,
3-{[6-morpholin-4-yl-2-(2-pyridin-2-yl-ethoxy)-pyrimidin-4-yl]-hydrazonomethyl}-benzoic acid N-methyl amide, or
(3-{[6-morpholin-4-yl-2-(2-pyridin-2-yl-ethoxy)-pyrimidin-4-yl]-hydrazonomethyl}-phenyl)-methanol,
N,N-Diethyl-4-{4-[N″-(3-methyl-benzylidene)hydrazino]-6-morpholin-4-yl-pyrimidin-2-yl}-butyramide,
4-{4-[N′-(3-Methyl-benzylidene)-hydrazino]-6-morpholin-4-yl-pyrimidin-2-yl}-1-(4-methyl-piperazin-1-yl)-butan-1-one,
4-{4-[N′-(3-Methyl-benzylidene)-hydrazino]-6-morpholin-4-yl-pyrimidin-2-yl}-N-pyridin-4-ylmethyl-butyramide,
4-{4-[N′-(3-Methyl-benzylidene)-hydrazino]-6-morpholin-4-yl-pyrimidin-2-yl}-N-pyridin-4-yl-butyramide.
2-{4-[N′-(3-Methyl-benzylidene)-hydrazino]-6-morpholin-4-yl-pyrimidin-2-yloxy}-1-pyridin-2-yl-ethanol
6-(2-{4-[N′-(3-Methyl-benzylidene)-hydrazino]-6-morpholin-4-yl-pyrimidin-2-yloxy}-ethyl)-pyridin-3-ol
6-(2-{4-[N′-(3-Hydroxymethyl-benzylidene)-hydrazino]-6-morpholin-4-yl-pyrimidin-2-yloxy}-ethyl)pyridin-3-ol

42. A method for treating or preventing a disorder associated with excessive bone loss, the method comprising administering to a patient in need thereof a compound according to claim 1, a composition comprising an effective amount of a compound according to claim 1, a compound according to formula (I) as described in any one of claims 2-41, or a composition according to any one of claims 2-41.

43. The method according to claim 42, wherein the disorder is selected from the group consisting of periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers)

44. The method according to claim 42 or 43, the method further comprising administering another therapeutic agent.

45. The method according to claim 44, wherein the other therapeutic agent is selected from the group consisting of: anti-resporptive agents, non-steroidal anti-inflammatory agents, steroids, and analgesics.

46. The method according to claim 45, wherein the anti-resporptive agent is selected from the group consisting of progestins, polyphosphonates, bisphosphonate(s), estrogen agonists/antagonists, estrogen, estrogen/progestin combinations, and estrogen derivatives.

47. The method according to claim 46, wherein the estrogen derivative is estrone, estriol or 17α,17β-ethynyl estradiol.

48. A method for inhibiting osteoclast formation in a pre-osteoclast cell the method comprising contacting the cell with a compound according to claim 1, a composition comprising an effective amount of a compound according to claim 1, a compound according to formula (I) as described in any one of claims 2-41, or a composition according to any one of claims 2-41.

49. A compound selected from the group consisting of: 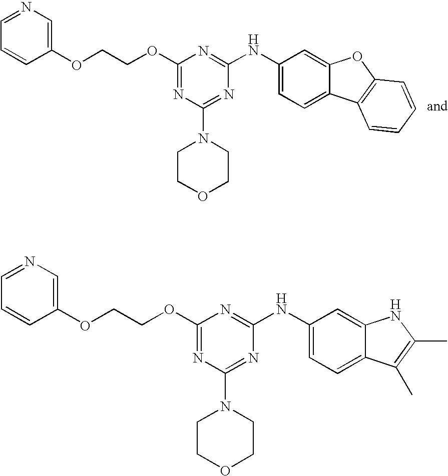
or a pharmaceutically acceptable salt, solvate, clathrate, or prodrug thereof.

50. A compound of formula (I′): 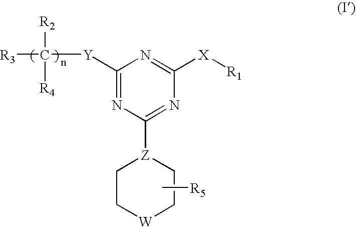
wherein
R₁ is 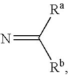
aryl, or heteroaryl;
each of R₂, R₄, and R₅, independently, is R^{c}, halogen, nitro, nitroso, cyano, azide, isothionitro, SR^{d}, or OR^{c};
R₃ is R^{c}, alkenyl, alkynyl, aryl, heteroaryl, cyclyl, heterocyclyl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{e}, COR^{c}, C(O)OR^{c}, or C(O)NR^{c}R^{d};
n is 0, 2, 3, 4, 5, 6, or 7;
X is O, S, S(O), S(O₂), or NR^{c};
Y is a covalent bond, CH₂, C(O), C═N—R^{c}, C═N—OR^{c}, C═N—SR^{c}, O, S, S(O), or S(O₂);
Z is N; and
W is O, S, S(O), S(O₂), NR^{c}, or NC(O)R^{c};
in which each of R^{a} and R^{b}, independently, is H, alkyl, aryl, heteroaryl; and each of R^{c} and R^{d}, independently, is H, alkyl, or alkylcarbonyl
or a pharmaceutically acceptable salt, solvate, clathrate, or prodrug thereof.

51. A composition comprising an effective amount of a compound of formula (I′): 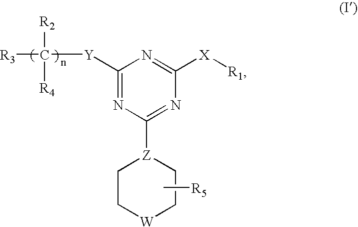
wherein
R₁ is 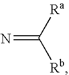
aryl, or heteroaryl;
each of R₂, R₄, and R₅, independently, is R^{c}, halogen, nitro, nitroso, cyano, azide, isothionitro, SR^{c}, or OR^{c};
R₃ is R^{c}, alkenyl, alkynyl, aryl, heteroaryl, cyclyl, heterocyclyl, OR^{c}, OC(O)R^{c}, SO₂R^{c}, S(O)R^{c}, S(O₂)NR^{c}R^{d}, SR^{c}, NR^{c}R^{d}, NR^{c}COR^{d}, NR^{c}C(O)OR^{d}, NR^{c}C(O)NR^{c}R^{d}, NR^{c}SO₂R^{d}, COR^{c}, C(O)OR^{c}, or C(O)NR^{c}R^{d};
n is 0, 1, 2, 3, 4, 5, 6, or 7;
X is O, S, S(O), S(O₂), or NR^{c};
Y is a covalent bond, CH₂, C(O), C═N—R′, C═N—OR^{c}, C═N—SR^{c}, O, S, S(O), S(O₂), or NR^{c};
Z is CH; and
W in which each of R^{a} and R^{b}, independently, is H, alkyl, aryl, heteroaryl; and each of R^{c} and R^{d}, independently, is H, alkyl, or alkylcarbonyl
or a pharmaceutically acceptable salt, solvate, clathrate, or prodrug thereof.

52. A compound selected from the group consisting of: 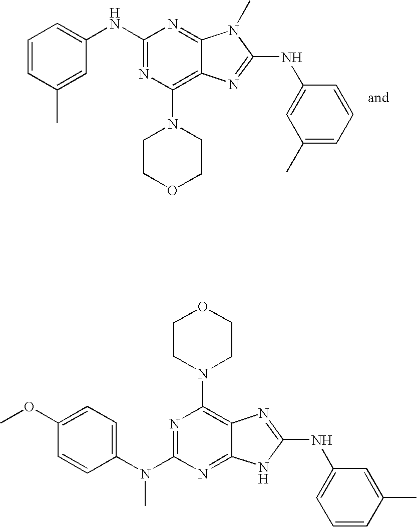
or a pharmaceutically acceptable salt, solvate, clathrate, or prodrug thereof.

53. A compound of formula (I″): 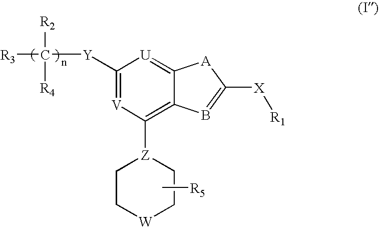
wherein
R₁ is aryl or heteroaryl;
each of R₂ and R₄, independently, is H, halogen, CN, alkyl, OR^{a}, or NR^{a}R^{b};
R₃ is H, halogen, CN, alkyl, alkenyl, alkynyl, aryl, heteroaryl, cyclyl, heterocyclyl, OR^{a}, OC(O)R^{a}, OC(O)NR^{a}R^{b}, NR^{a}R^{b}, NR^{a}C(O)R^{b}, NR^{a}S(O)R^{b}, NR^{a}S(O)₂R^{b}, NR^{a}C(O)NR^{b}R^{c}C NR^{a}C(S)NR^{b}R^{c}, NR^{a}C(NR^{b})NR^{c}R^{d}, NR^{a}C(O)OR^{b}, S(O)NR^{a}R^{b}, S(O)₂NR^{a}R^{b}, S(O)R^{a}, S(O)₂R^{a}, C(O)R^{a}, C(O)OR^{a}, or C(O)NR^{a}R^{b};
R₅ is H or alkyl;
n is 0, 1, 2, 3, 4, 5, or 6;
A is O, S, S(O), S(O)₂, or NR^{e};
B is N or CR^{f};
X is O, S, S(O), S(O)₂, NR^{e}, or C(O);
Y is a covalent bond, C(O), C═NR^{a}, O, S, S(O), S(O)₂, or NR^{e};
Z is N or CH;
each of U and V, independently, is N or CR; and
W is O, S, or NR^{e};
in which each of R^{a}, R^{b}, R^{c}, and R^{d}, independently, is H, alkyl, aryl, heteroaryl, cyclyl, or heterocyclyl; R^{e} is H, alkyl, aryl, acyl, or sulfonyl; and R^{f} is H, alkyl, aryl, acyl, sulfonyl, alkoxyl, amino, ester, amide, CN, or halogen.

54. A composition comprising an effective amount of a compound of formula (I″): 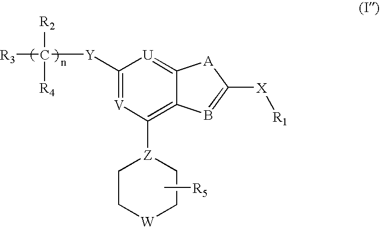
wherein
R₁ is aryl or heteroaryl;
each of R₂ and R₄, independently, is H, halogen, CN, alkyl, OR^{a}, or NR^{a}R^{b};
R₃ is H, halogen, CN, alkyl, alkenyl, alkynyl, aryl, heteroaryl, cyclyl, heterocyclyl, OR^{a}, OC(O)R^{a}, OC(O)NR^{a}R^{b}, NR^{a}R^{b}, NR^{a}C(O)R^{b}, NR^{a}S(O)R^{b}, NR^{a}S(O)₂R^{b}, NR^{a}C(O)NR^{b}R^{c}, NR^{a}C(O)NR^{b}R^{c}, NR^{a}C(NR^{b})NR^{c}R^{d}, NR^{a}C(O)OR^{b}, S(O)NR^{a}R^{b}, S(O)₂NR^{a}R^{b}, S(O)R^{a}, S(O)₂R^{a}, C(O)R^{a}, C(O)OR^{a}, or C(O)NR^{a}R^{b};
R₅ is H or alkyl;
n is 0, 1, 2, 3, 4, 5, or 6;
A is O, S, S(O), S(O)₂, or NR^{e};
B is N or CR^{f};
X is O, S, S(O), S(O)₂, NR^{e}, or C(O);
Y is a covalent bond, C(O), C═NR^{a}, O, S, S(O), S(O)₂, or NR^{e};
Z is N or CH;
each of U and V, independently, is N or CR; and
W is O, S, or NR^{e};
in which each of R^{a}, R^{b}, R^{c}, and R^{d}, independently, is H, alkyl, aryl, heteroaryl, cyclyl, or heterocyclyl; R^{e} is H, alkyl, aryl, acyl, or sulfonyl; and R^{f} is H, alkyl, aryl, acyl, sulfonyl, alkoxyl, amino, ester, amide, CN, or halogen
or a pharmaceutically acceptable salt, solvate, clathrate, or prodrug thereof.

55. A method for treating or preventing a disorder associated with excessive bone loss, the method comprising administering to a patient in need thereof a compound according to claim 49 or 52, a composition comprising an effective amount of a compound according to claim 49 or 52, a compound according to formula (I′) or (I″) as described in any one of claims 50 or 53, or a composition according to any one of claims 51 or 54.

56. The method according to claim 55, wherein the disorder is selected from the group consisting of periodontal disease, non-malignant bone disorders (such as osteoporosis, Paget's disease of bone, osteogenesis imperfecta, fibrous dysplasia, and primary hyperparathyroidism) estrogen deficiency, inflammatory bone loss, bone malignancy, arthritis, osteopetrosis, and certain cancer-related disorders (such as hypercalcemia of malignancy (HCM), osteolytic bone lesions of multiple myeloma and osteolytic bone metastases of breast cancer and other metastatic cancers).

57. A method for inhibiting osteoclast formation in a pre-osteoclast cell the method comprising contacting the cell with a compound according to claim 49 or 52, a composition comprising an effective amount of a compound according to claim 49 or 52, a compound according to formula (I′) or (I″) as described in any one of claims 50 or 53, or a composition according to any one of claims 51 or 54.
